(19) United States
(12) Patent Application Publication
(10) Pub. No.: US 2024/0239787 A1
(43) Pub. Date: Jul. 18, 2024

(54) COMPOUNDS FOR USE IN THE TREATMENT OF CANCER

(71) Applicant: Nanyang Technological University, Singapore (SG)

(72) Inventors: Bengang XING, Singapore (SG); Cong Thang DO, Singapore (SG)

(21) Appl. No.: 18/556,711

(22) PCT Filed: May 6, 2022
(86) PCT No.: PCT/SG2022/050282

§ 371 Date: Oct. 23, 2023

(30) Foreign Application Priority Data
May 6, 2021 (SG) 10202104740P

(51) Int. Cl. C07D 417/14; A61K 31/428; A61K 31/454; A61K 31/5517; A61P 35/00; C07D 417/12; C07D 495/14

(52) U.S. Cl. CPC C07D 417/14; A61K 31/428; A61K 31/454; A61K 31/5517; A61P 35/00; C07D 417/12; C07D 495/14

(57) ABSTRACT

Disclosed herein are two compounds that are used together to form a PROTAC molecule in situ. Thus, there is a compound of formula Ia and a compound of formula Ib: where R1, R2, L1, L2, X and Y are described herein.

### FIELD OF INVENTION

The current invention relates to the use of two compounds where at least one of the compounds can be activated in situ, such that the two compounds react together using Click chemistry at a site of action to provide a therapeutic compound. Also described herein are further uses of said compounds and their manufacture.

### BACKGROUND

The listing or discussion of a prior-published document in this specification should not necessarily be taken as an acknowledgement that the document is part of the state of the art or is common general knowledge.

Among the processes to achieve promising activity against disease development (e.g. tumours, etc.), effective recognition of therapeutic reagents by the targeted biomolecules (e.g. proteins, etc.) usually play a critical role in sustaining the potency and efficacy desired in the treatment. Such targeting identification can not only achieve effective validation of the pharmacokinetic and pharmacodynamic processes of drug treatment, it can also provide detailed understanding towards mechanism-of-action, thus facilitating new drug screening and discovery. Therefore, integration of modern chemical biology and drug discovery to identify small functional molecules and their selective manipulation of activities toward target proteins for first-in-class drug development is highly desirable. As an important signaling pathway, circadian clock regulates daily oscillation of almost every cell rhythmic behaviour and physiology in mammals. These conserved biological timekeepers are self-sustaining, feedback-loop based complex systems which synchronizes day/night cycle and the corresponding changes in environmental conditions with biological functioning. Disruption of normal circadian rhythmicity is associated with a variety of disorders and often directly lead to pathological states such as Alzheimer's, cardiovascular, gastrointestinal, psychological disease and tumorigenesis. Generally, the molecular mechanism of the circadian clock is mainly based on the interlocked transcriptional-translational feedback loops. The core-loop is the heterodimer of two transcriptional factors, BMAL1 and CLOCK, and activate the expression of Cryptochrome (Cry) and Period (Per) genes. After the post-translational modification and cytosol accumulation processes, Per and Cry proteins inhibit CLOCK-BMAL1 functions in nucleus, resulting in rhythmic gene expression. The degradation of Per and Cry by 26S proteasomal pathway release the inhibition and start a new transcription cycle. The indispensable post-translational modification of the feedback loops components is therefore considered as a crucial layer in clock regulation. Particularly, phosphorylation of clock proteins by various kinases have been proven to modify the period length. Therefore, a complete understanding and characterization of the responsible kinases for each clock protein is necessary for controlling the circadian network as well as implications in biological monitoring.

Recently, chemical modulation of clock proteins via small molecules have emerged as a time- and dose-dependent strategies for tweaking circadian rhythmicity. Unlike conventional genetic approaches, small molecular usage reduces risks factors such as fatality, pleiotropy, and functional redundancy. These small modulators have promoted further investigation of post-translational mechanism underlying clock-regulatory pathways as well as have been shown to have therapeutics functions. For instance, longdaysin was characterized as modulators that inhibit CKIα-mediated phosphorylation, stimulating the degradation of a clock protein PER1 and drastically lengthening the circadian period. Other small molecules such as SR9009 and SR9011, two agonists of REV-ERBs (secondary clock component) revealed their anti-cancer effects in selective manner. However, the correlation of circadian regulation with therapeutics effects, specifically anti-cancer potency, have not been clearly addressed. Extensive explorations are still desired in the field.

Hypoxia, the state of low oxygen level, is a common feature of most tumours and frequently represents the pernicious effects of cancer progression, therapeutic resistance, uncontrolled metastasis, and poor prognosis. The cellular response to oxygen deficiency is mainly mediated by hypoxia-inducible factors (HIFs), one family of crucial transcription factors orchestrating the expression of various essential genes involved in manipulation of epigenetic plasticity and cancer hallmark's acquisition to adapt hostile homeostasis in tumours. Apart from the direct effect on gene expression in hypoxic tumours, these HIF factors and hypoxia microenvironment can also influence the epigenetic mechanisms by exerting their inheritable molecular regulation. Moreover, recent evidence further demonstrated the contribution of epigenetic alterations to the hypoxia response (Zheng, F. et al., Nat. Commun. 2021, 12, 1341; Semenza, G., Nat. Rev. Cancer 2003, 3, 721-732; and Chen, Y. et al., J. Clin. Invest 2018, 128, 1937-1955). Therefore, the clear interpretation of intrinsic correlation between hypoxia and epigenetic regulations are of high significance to establish plausible cancer hallmarks for effective therapeutic developments. However, much detail remains to be further clarified so far.

BRD4, as a critical BET family member, can recognize acetylated histones and recruit transcription factors as well as epigenetic mediators to regulate gene replication and transcription. Dysfunction of BRD4 protein has been intricately linked to the malignant development of various tumours, thus emerging as a promising epigenetic target for cancer treatment (Crawford, N. P. et al., Proc. Natl. Acad. Sci. USA 2008, 105, 6380-6385; Zuber, J. et al., Nature 2011, 478, 524-528; and Zanconato, F. et al., Nat. Med. 2018, 24, 1599-1610). So far, BRD4 inhibition or degradation has been established to effectively constraint the malignant progression and distant metastasis of tumours (Filippakopoulos, P. et al., Nature 2010, 468, 1067-1073; da Motta, L. et al., Oncogene 2017, 36, 122-132; Zhou, S. et al., Oncogenesis 2020, 9, 33; Yin, M. et al., Nat. Commun. 2020, 11, 1833; Winter, G. E. et al., Science 2015, 348, 1376-1381; and Qin, C. et al., J. Med. Chem. 2018, 61, 6685-6704).

Among the different strategies, proteolysis targeting chimeras (PROTACs) are typical heterobifunctional molecules consisting of a ligand specific for an intracellular protein of interest (POI) associating with an E3 ubiquitin ligase recruiting moiety through an appropriate linker that triggers the ubiquitination and subsequent degradation of target proteins in the proteasome (Zengerle, M., Chan, K.-H. & Ciulli, A., ACS Chem. Biol. 2015, 10, 1770-1777; Sakamoto, K. M. et al., Proc. Natl. Acad. Sci. U.S.A. 2001, 98, 8554-8559; and Bondeson, D. P. et al., Nat. Chem. Biol. 2015, 11, 611-617). Unlike traditional drug design through occupancy-driven inhibition, the PROTAC technology exhibits great pharmacological merits with less susceptibility to point mutations in target proteins. Moreover, such ubiquitin-mediated protein degradation can abrogate or manipulate all the relative downstream regulators for disease cessation. The maximized drug potency can be fully potentiated not only for the concerns of therapeutic resistance, but also for addressing multifunctional drugs or targets with unknown functions (Pettersson, M. & Crews, C. M., Drug Discov. Today Technol. 2019, 31, 15-27).

However, PROTACs development requires extensive investigation of the physicochemical properties of POI, E3 ligase ligand, and linkers, by evaluating the prerequisite architecture and structure-activity relationship. Disparity or alteration of any one of these three elements can lead to substantial diminution in activity (Zorba, A. et al., Proc. Natl. Acad. Sci. USA. 2018, 115, E7285-E7292; Zheng, M., J. Med. Chem. 2021, 64, 7839-7852; Troy, A. B., James, J. L. C., & Michael, D. B., J. Med. Chem. 2021, 64, 8042-8052; and Smith, B. E. et al., Nat. Commun. 2019, 10, 131). As both ends of the PROTAC are connected via a linker, the exact nature of which needs to be carefully chosen to ensure an optimal ADMET profiling (Gerry, C. J. & Schreiber, S. L., Nat. Chem. Biol. 2020, 16, 369-378; Guo, W. H. et al., Nat. Commun. 2020, 11, 4268; and Dale, B. et al., Nat. Rev. Cancer 2021, 21, 638-654). Typically, the high molecular weight which deviates from the conventional “rule of five” for drug-like molecule properties may limit their cellular uptake, compromise bioavailability and pharmacokinetics, therefore constraining its application in systematic practices (Pfaff, P. et al., ACS Cent. Sci. 2019, 5, 1682-1690; Gabizon, R. et al., J. Am. Chem. Soc. 2020, 142, 11734-11742; and Imaide, S. et al., Nat. Chem. Biol. 2021, 17, 1157-1167). On the other hand, since POI is degraded, a subset of the protein functions are also suppressed. Such deleterious consequences might interfere with the regular protein functions in normal cells with uncontrolled degradation of the protein when no cell-selective manner of molecules is being assisted. Therefore, intracellular accumulation of heterobifunctional molecules needs to be monitored carefully to minimize off-target response. It can be achieved by controlling its cell-type selectivity or by precise assessment of the appropriate compound concentration to regulate proteolysis. To comprehend and resolve target specificity, the incorporation of affinity ligands or reporters into the conventional architecture of PROTACs are developing progressively, but it brings additional complexity to the compounds which might hinder their application towards clinical translation (Kounde, C. S. & Tate, E. W., J. Med. Chem. 2020, 63, 15483-15493; Liu, J. et al., J. Am. Chem. Soc. 2021, 143, 7380-7387; and He, S., Angew. Chem. Int. Ed. Engl. 2021, 60, 23299-23305).

Therefore, there exists a need for substantial consideration of the design of PROTACs to bring historically undruggable protein into play for therapeutic benefits.

### DRAWINGS

FIG. 1 depicts the synthesis route for T44 and T120 by covalent conjugation of pomalidomide (one typical ligand for E3 ligase) and GO289 (one recognition molecule for CK2α).

FIG. 2 depicts the circadian protein CK2α degradation upon the incubation of (a) T44 (0.1 μM and 1 μM); and (b) T120 (0.1 μM and 1 μM) in live cells.

FIG. 3 depicts the concentration-dependent test on circadian protein CK2α degradation upon incubation of T44 and T120 (0.1 μM and 1 M) in live cells.

FIG. 4 depicts the Enzymatic Click Induced Proteolysis Targeting Chimera (ENCTAC) degradation.

FIG. 5 depicts the first-in-class and personalized ENCTAC-based PROTAC strategy for selective and specific circadian protein degradation in dynamic and complex live conditions.

FIG. 6 depicts the chemical synthetic enzyme responsive peptide PROTAC (ENCTAC) moieties (for pomalidomide and GO289) with different linkage distance.

FIG. 7 depicts furin ENCTAC degradation.

FIG. 8 depicts cathepsin B (CtsB) ENCTAC degradation.

FIG. 9 depicts nitroreductase (NTR) enzyme ENCTAC degradation.

FIG. 10 depicts the illustration of ENCTAC performances in hypoxic condition for selective degradation of BRD4 epigenetic proteins.

FIG. 11 depicts the chemical synthetic enzyme responsive peptide PROTAC (ENCTAC) moieties (for pomalidomide and JQ1) with different linkage distance.

FIG. 12 depicts the enzymatic click-induced formation of heterobifunctional degraders of BRD4. (a) Chemical structure of glutathione (GSH) responsive CRBN ligand (J266), GSH and NTR responsive CRBN ligand (JW4), cleaved-J266, 2-cyanobenzothiazole (CBT) linked-BRD4 targeting ligand (JQ1-CBT), click-induced BRD4 degrader (J252); (b) Liquid chromatography-mass spectrometry (LC-MS) spectra of NTR enzyme uncaging JW4 (10 μM) to form J266 at different time points in NTR (40 μg/mL) dissolved in PBS (pH 7.4, 10 mM); (c) Selectivity of JW4 towards a broad range of enzymes and chemical agents with or without NADH; (d) Time-dependent LC-MS spectra of click-J252 formation under reducing reagent (tris(2-carboxyethyl) phosphine (TCEP)) that cleaves the J266 to induce cleaved-J266 before click reaction with JQ1-CBT in the solution; and (e) Ratio of peak areas of click-J252 and cleaved-J266 products to J266 over multiple time points in (d).

FIG. 13 depicts (a) LC-MS spectra of NTR uncaging JW4 (10 μM) to form J266 in different concentrations of NTR enzyme in PBS (pH 7.4, 10 mM); (b) LC-MS spectra of NTR uncaging of varied concentrations of JW4 substrate to form J266 in NTR enzyme (40 μg/mL) in 3 h; (c) ratio-metric peak areas of J266 relative to JW4 over multiple time points; (d) ratio-metric peak areas of J266 relative to JW4 over multiple concentrations of NTR enzyme for 2 h; (e) LC-MS spectra of time-dependent NTR uncaging J268 (10 M) to form J264 in NTR enzyme in PBS (pH 7.4, 10 mM); (f) ratio-metric peak areas of J264 relative to J268 over multiple time points in (e); (g) LC-MS spectra of NTR concentration-dependent uncaging J268 (10 μM) to form J264 in PBS (pH 7.4, 10 mM); and (h) ratio-metric peak areas of J266 relative to JW4 over multiple concentrations of NTR enzyme during 2 h.

FIG. 14 depicts the LC-MS spectra of one-pot, continuous NTR uncaging of JW4 (10 μM) in NTR enzyme (40 μg/mL), and subsequent J266 cleavage by TCEP to form click product-J252 in the presence of JQ1-CBT (10 μM) (Reference spectra of pre-synthesized J266 and J252 were added).

FIG. 15 depicts that the heterobifunctional degraders efficiently degrade BRD4 protein. (a) Docking simulation of J252 interacting with CRBN and BRD4 proteins. The position of luciferin-based component shows no steric collision with BRD4 or CRBN; and (b) Western blot analysis of BRD4 protein after treatment with J252, and ARV-825 at the indicated concentrations for 24 h, and β-Tubulin as the internal control.

FIG. 16 depicts the docking simulation of (a) J242 and (b) T208 in interaction with CRBN and BRD4 proteins, and western blot analysis of BRD4 protein after treatment with J252 and ARV-825 at the indicated concentrations for 24 h, and β-Tubulin as internal control.

FIG. 17 depicts the hypoxia activated degradation of epigenetic BRD4 protein. (a) Illustration of intracellular hypoxic enzyme uncaging and formation of degrader (click-J252) and degradation of BRD4; (b) Western blot analysis of BRD4 protein levels after treatments with GSH cleavable J266 (6 h) and JQ1-CBT (12 h) at indicated concentration and different time, GSH and NTR responsive JW4 (6 h) with JQ1-CBT (12 h) at indicated concentration and time under hypoxia in HEK293T cells; (c) Analysis of BRD4 protein levels treatment under combination of different control conditions for 12 h; (d) Western blot analysis of BRD4 protein levels after treatments with J266 (6 h) and JQ1-CBT (12 h) or JW4 (6 h) and JQ1-CBT (12 h) in time and concentration-dependent examinations of HeLa cancer cells, analysis of BRD4 protein levels after treatments with JQ1 (12 h) in concentration-dependent manner of hypoxia Hela cells; JW4 (6 h) and JQ1-CBT (12 h) in normoxia Hela cells; (e) Extended concentration of degraders demonstrating the “Hook effect” during incubation under hypoxia. β-tubulin was used as the internal control; and (f) Degradation level of BRD4 protein over varied concentrations as indicated in (e). Values represent triplicate means±SD, normalized to non-treated cells and baseline-corrected using immunoblots.

FIG. 18 depicts the immunoblot for BRD4 levels of Hela cells after 12 h treatment with separated ENCTAC fragments of JW4 or JQ1-CBT. Immunoblot for BRD4 levels of MDA-MB-231, 4T1, B16F10 cells treated with combination of JW4 and JQ1-CBT under hypoxia or normoxia. β-Tubulin was used as the internal control.

FIG. 19 depicts the in vitro degradation of epigenetic protein BRD4 under hypoxia condition, resulting in alteration of cellular microenvironment response and cell growth malfunction. (a) Confocal microscope fluorescent imaging of HIF-1α immunostaining (λₑₓ: 488 nm, λₑₘ: 520/30 nm) in hypoxia condition after treatments of different concentrations of JW4 and JQ1-CBT for ENCTAC degradation of BRD4 in Hela cells. Cells under normoxia condition were used as the control. The nucleus was stained with Hoechst (λₑₓ: 405 nm, λₑₘ: 450/30 nm), β-Tubulin was stained by fluorescent β-tubulin antibody (λₑₓ: 561 nm, λₑₘ: 590/30 nm); (b) Quantitative mean fluorescence intensity of HIF-1α after treatment and staining as indicated in (a); Western blot analysis of HIF-1α after treatment with different concentrations of (c) JW4 and JQ1-CBT or (d) separated JW4, JQ1-CBT, JQ1, J252 controls; (e) Western blot analysis of VEGF and CA9 levels after hypoxia activated ENCTAC treatment using JW4 and JQ1-CBT at the indicated concentrations; (f) Protein level of HIF-1α, VEGF, and CA9 proteins after hypoxia ENCTAC treatment and JQ1 inhibitor treatment over varied concentrations. Values represent the average of duplicates and the range as error bars, normalized to non-treated cells and baseline-corrected using immunoblots; (g) Immunoblots for c-Myc and GAPDH levels after hypoxia activated ENCTAC treatment using JW4 and JQ1-CBT at the indicated concentrations for 12 h; (h) Immunoblots for poly(ADP-ribose) polymerase (PARP) cleavage and glyceraldehyde 3-phosphate dehydrogenase (GAPDH) levels after similar treatment condition as indicated in (g); (i) Confocal microscope imaging of apoptosis cell death staining using Annexin V/Propodium Iodide (PI) (Annexin V, λₑₓ: 488 nm, λₑₘ: 520/30 nm; λₑₓ: 561 nm, λₑₘ: 590/30 nm). The cell under hypoxia without ENCTAC treatment was used as the control; and (j) Flow cytometry of apoptosis/necrosis-stained Hela cells under treatments of JW4, JQ1-CBT, or combination of JW4 and JQ1-CBT (10 μM). Quarter 1 (Q1) indicates the relative percentage of necrosis cells, Q2: late apoptosis cells, Q3: early apoptosis cells, and Q4: live cells.

FIG. 20 depicts the western blot analysis of VEGF and CA9 levels after (+)-JQ1 treatment at the indicated concentrations in hypoxia Hela cells.

FIG. 21 depicts that the in vivo degradation of BRD4 protein using ENCTACs manipulate hypoxic zebrafish development. (a) Schematic illustration of zebrafish early treatment with hypoxia activated ENCTACs; (b) Western blot analysis of BRD4 protein level in hypoxia zebrafish after treatment with different concentrations of ENCTAC molecules; (c) Western blot analysis of HIF-1α protein level in hypoxia zebrafish after treatment with different concentrations of ENCTAC molecules or with inhibitor JQ1; (d) Bright field images of zebrafish embryos under different conditions of indicated drug treatment at 36 h post fertilization (hpf); (e) Fluorescence microscope imaging of zebrafish larvae phenotype with blood vessel trackers (GFP) in wild type (WT), and VHL mutant (VHL^{-/-}) larvae with and without ENCTAC treatment. λₑₓ: 488 nm, λₑₘ: 520/30 nm; (f) Fluorescence intensity spectra of blood vessel alignment along the line indicated in (e); and (g) Statistical numbers of vascularization phenotypes in zebrafish larvae with and without ENCTAC treatments or with PROTAC, J252, or inhibitor JQ1 (10 μM).

FIG. 22 depicts the brightfield imaging of VHL mutant transgenic zebrafish or wide type zebrafish with different level of vascularization due to angiogenesis process. The strong, mild, and wide type were classified based on the order of blood vessels as indicated by the arrows.

FIG. 23 depicts the JW40 fluorescent dye for NTR detection in hypoxic zebrafish larvae. (a) LC-MS spectra of NTR uncaging of JW40 (10 μM) in NTR enzyme (40 μg/mL) to produce resorufin dye; (b) Fluorescent spectra of JW40 (10 M) in NTR enzyme (40 μg/mL) in phosphate buffered saline (PBS, pH 7.4, 10 mM) during 8 h incubation at 37° C. and JW40 (10 μM) without enzyme treatment; (c) Confocal imaging of VHL^{-/-} transgenic zebrafish or wide type zebrafish after 12 h treatment of JW40 (50 μM). VHL mutant larvae showed significant enhancement in red fluorescence due to NTR uncaging JW40. λₑₓ: 488 nm, λₑₘ: 520/30 nm; red: λₑₓ: 564 nm, λₑₘ: 610/30 nm; and (d) Fluorescence spectra of the incubation medium of VHL^{-/-} transgenic zebrafish or wide type zebrafish after 48 h treatment of JW40 (50 μM), showing an enhancement in resorufin signal which is attributed to greater NTR uncaging of the VHL/larvae.

FIG. 24 depicts the antitumour advantages of ENCTAC assisted BRD4 degradation. (a) Diagram of tumour xenograft establishment, treatment and analysis; (b) Fluorescence image of NTR activity in solid tumour. Near-infrared (NIR) fluorescence reporter was injected intra-tumorally and the image was taken 30 min later; (c) Immunoblots for BRD4, c-MYC, HIF-1α and GAPDH by using tumour lysates from mice treated with these molecules 2 times at 4 h and 8 h of JW4 and JQ1-CBT (5 mg/kg) mixture, and (+)-JQ1 (5 mg/kg), compared with a vehicle-treated control; (d) Tumour volume (means±SD) of vehicle-treated mice (n=3) or mice treated with JW4 and JQ1-CBT mixture (5 mg/kg; n=4), and JQ1 treated mice (n=3) for 5 days; (e) Relative tumour growth rate under different treatments as indicated in (c) for 5 days; (f) Immunohistochemistry for CD31 of a representative tumour of an ENCTAC-treated, (+)-JQ1 treated and a control-treated mouse; and (g) Quantification of three independent CD31 areas/4′,6-diamidino-2-phenylindole (DAPI) areas in (d).

### DESCRIPTION

We have now discovered, surprisingly, that it is possible to deliver a PROTAC molecule to the desired site of action as two separate component compounds that may then be metabolised at or close to the site of action (e.g. within a target cell) and then undergo a Click reaction to provide the PROTAC molecule. Advantages of this route may include reducing the toxicity of the PROTAC molecule and/or allowing more convenient dosing strategies (e.g. oral administration).

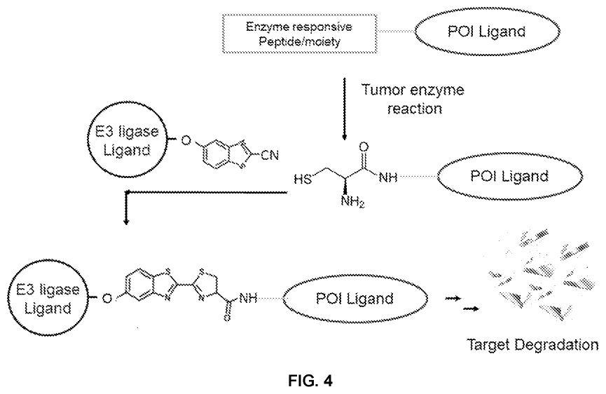

The current invention relates to an Enzymatic Click Inducible Proteolysis Targeting Chimera (EnC-TAC, or ENCTAC) that can achieve targeted PROTAC degradation that specifically occurs in a tumour environment. This is different from conventional approaches using PROTAC degradation. Rather than chemically connect a POI (Protein of Interest) recognizing moiety with an E3 ligase recruiter in a single molecule that is then administered as a whole to a subject, the current invention makes use of a different strategy. That is, the POI recognizer and E3 ligase recruiter are modified with specific tumour-responsive moieties (e.g. pH, radical, light irradiation or tumour enzyme-responsive small molecules, or peptide sequences etc.) and 2-cyanobenzothiazole, respectively (or vice versa). This strategy is demonstrated herein using the tumor specific protease enzyme (e.g. nitroreductase, furin, cathepsin B, caspases etc.). Typically, as shown in FIG. 4, upon the effective cell penetration of the two separate chemically modified fragments (e.g. an E3 ligase ligand such as pomalidomide etc., and a POI ligand) within the tumor cells, the tumor specific environment causes a reaction (e.g. specific enzyme reactions including protease and other enzymes etc.) that leads to the cleavage of the recognition sequence that orthogonally induces covalent cross-linking between the exposed cysteine and CBT (using Click chemistry inside the cell/tumour environment), thus achieving intrinsic PROTAC trapping within the tumour environment for targeted degradation of the desired POI. This strategy enables selective protein degradation in a tumour area, with no need for the conjugation of affinity ligands. In addition, the overall process does not require one to chemically prepare the entire molecule before administration—it allows one instead to provide two smaller fragments (with at least one of the fragments carrying enzyme-cleavable groups) for administration to a subject. The use of two smaller fragments of the desired PROTAC may assist delivery of the two components to the site of action and avoids the need to completely form the PROTAC in the laboratory, thus minimizing the synthetic route and also the limited cell penetration that large molecular weight PROTAC molecules suffer from. More importantly, the enzyme responsive cross-linking product may demonstrate a fluorescent property, which can provide a great opportunity to precisely quantify the PROTAC and visualize the proteasome ablation of a cancer POI in real-time.

Thus, the application of the current invention requires two components, a compound of formula Ia and a compound of formula Ib, or pharmaceutically acceptable salts or solvates thereof, that are used in combination to generate a desired PROTAC compound in situ at a desired site of action (e.g. within a cancer cell).

Thus, in a first aspect of the invention, there is provided a compound of formula Ia:

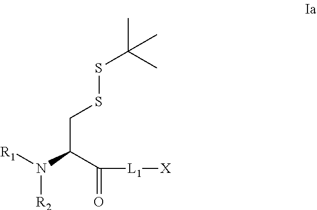

where:
R₁ is H or one or more amino acids selected from Y, A, W, H, L, Q, T, D, E, and more particularly R, V, F, and K, where a terminal amino acid of the one or more amino acids has a free NH₂ group or a NHAc group;
R₂ is H or

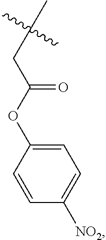 [SMILES: *CC(=O)Oc1ccc([N+](=O)[O-])cc1]

where the wiggly line represents the point of attachment to the rest of the molecule;
L₁ is a linking group; and
X is an E3 ligase ligand or a protein of interest ligand,
or a pharmaceutically acceptable salt or solvate thereof, provided that at least one of R₁ and R₂ is not H.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a compound” includes mixtures of two or more such compounds, reference to “a composition” includes mixtures of two or more such compositions, and the like.

In embodiments of the first aspect of the invention, R₁ may be selected from the group including, but not limited to AcDEVD-, AcYVAD-, AcWEHD-, AcVDVAD-, AcLEVD-, AcLEHD-, AcVQVD-, ACLETD-, DEVD-, YVAD-, WEHD-, VDVAD-, LEVD-, LEHD-, VQVD-, LETD-, H, RVRR- or Ac-FK-. In embodiments of the invention that may be mentioned herein, R₁ may be selected from H, RVRR- or Ac-FK-.

In embodiments of the invention, L₁ may be attached to X by way of a nitrogen atom on X or by way of an oxygen atom on X.

Thus, in embodiments of the first aspect of the invention where L₁ is attached to a nitrogen atom on X, it may be selected from:
a bond; or

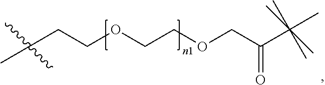

where the wiggly line represents the point of attachment to the carbonyl group next to L₁ and the crossed line represents the point of attachment to X and n1 represents from 0 to 5, such as from 0 to 3, such as 1 to 3; or

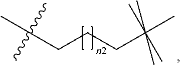

where the wiggly line represents the point of attachment to the carbonyl group next to L₁ and the crossed line represents the point of attachment to X and n2 represents from 0 to 10, such as 1 to 5, such as 1.

For example, where L₁ is attached to a nitrogen atom on X, X may be selected from the group including, but not limited to:

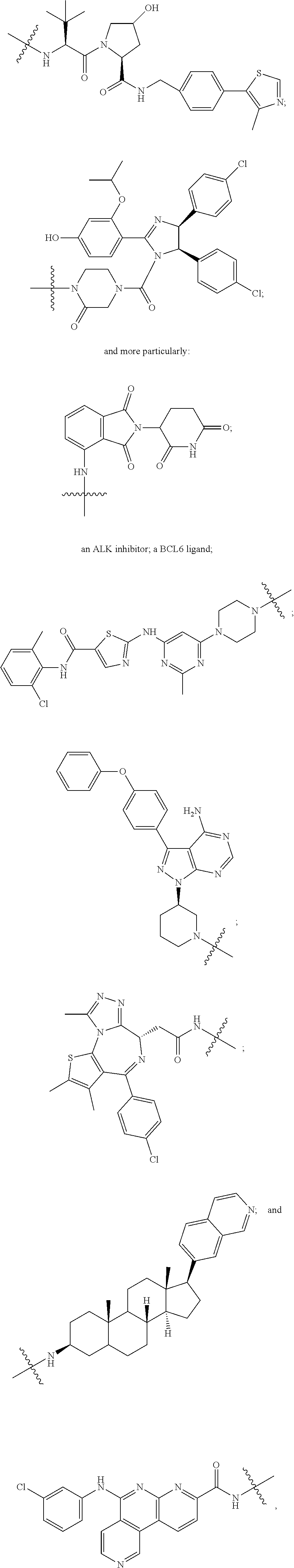

where the wiggly line represents the point of attachment to L₁.

In yet more particular embodiments of the invention where L₁ is attached to a nitrogen atom on X, X may be selected from:

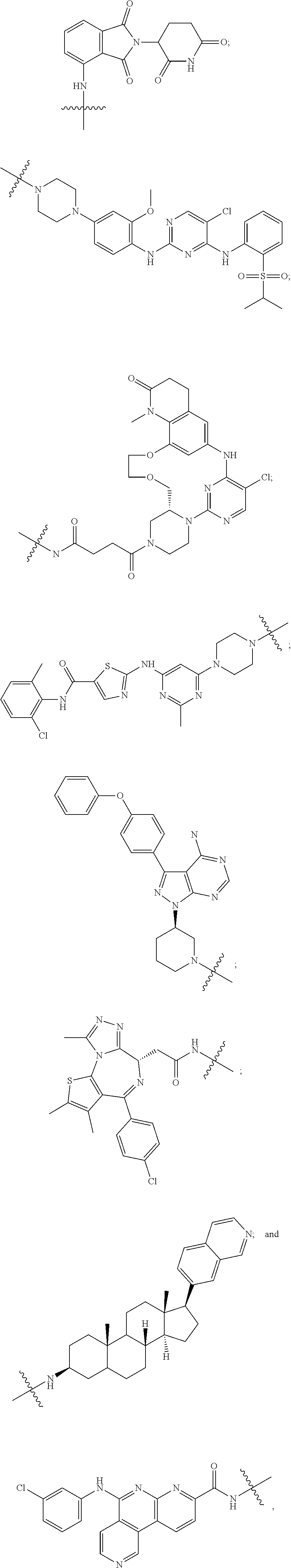

where the wiggly line represents the point of attachment to L₁.

Examples of the compounds of formula Ia that may be mentioned herein include, but are not limited to:

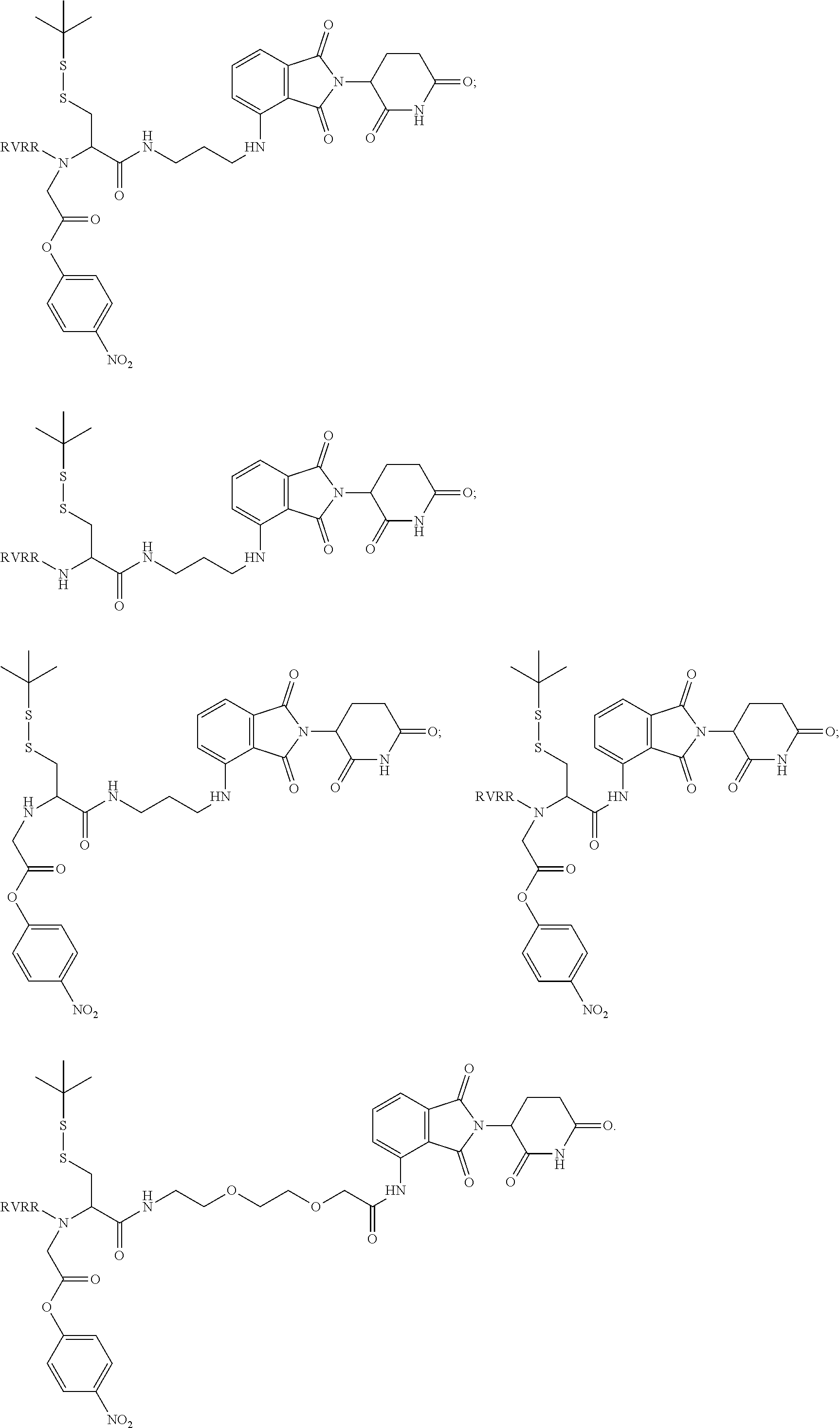

As will be appreciated, the compounds of formula Ia above may also be provided as pharmaceutically acceptable salts or solvates thereof.

In alternative embodiments of the invention, L₁ may be attached to an oxygen atom on X, in which case, it may be selected from:
a bond;

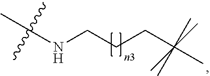

where the wiggly line represents the point of attachment to the carbonyl group next to L₁ and the crossed line represents the point of attachment to X and n3 represents from 1 to 5, such as from 1 to 3;

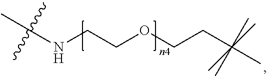

where the wiggly line represents the point of attachment to the carbonyl group next to L₁ and the crossed line represents the point of attachment to X and n4 represents from 0 to 10, such as 1 to 5, such as 1; or

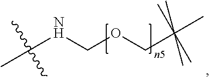

where the wiggly line represents the point of attachment to the carbonyl group next to L₁ and the crossed line represents the point of attachment to X and n5 represents from 0 to 10, such as 1 to 5, such as 1 to 2.

In embodiments where L₁ is attached to an oxygen atom on X, X may be selected from the list including, but not limited to:

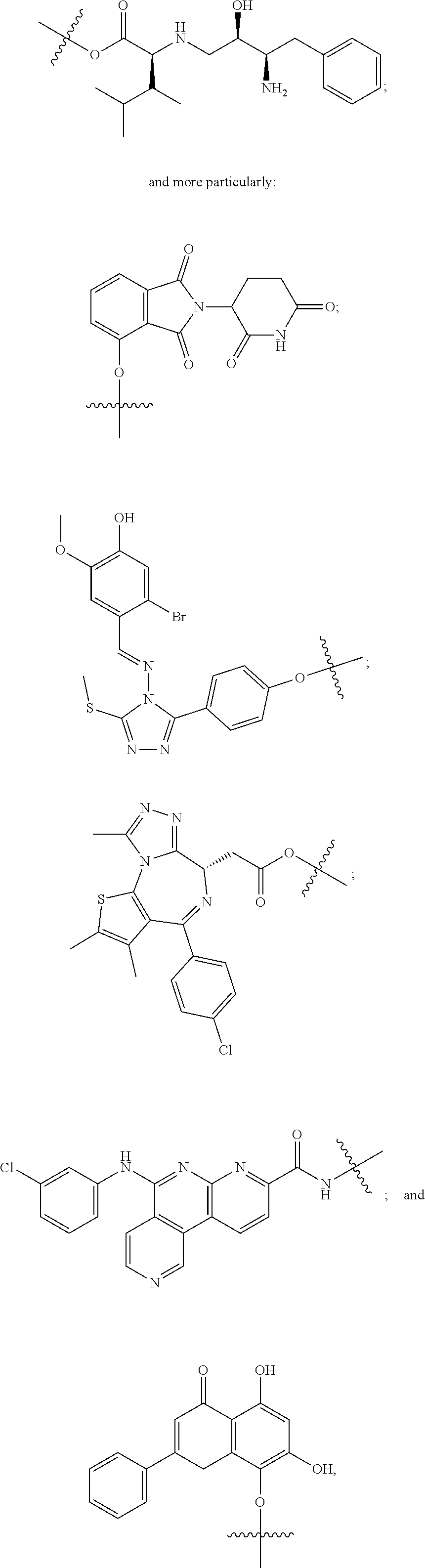 [SMILES: *OC(=O)[C@@H](NC[C@@H](O)[C@H](N)Cc1ccccc1)C(C)C(C)C ; *Oc1cccc2c1C(=O)N(C1CCC(=O)NC1=O)C2=O ; *Oc1ccc(-c2nnc(SC)n2/N=C/c2cc(OC)c(O)cc2Br)cc1 ; *OC(=O)C[C@@H]1N=C(c2ccc(Cl)cc2)c2c(sc(C)c2C)-n2c(C)nnc21 ; *NC(=O)c1ccc2c(n1)nc(Nc1cccc(Cl)c1)c1ccncc12 ; *Oc1c(O)cc(O)c2c1CC(c1ccccc1)=CC2=O]

where the wiggly line represents the point of attachment to L₁.

In more particular embodiments of the invention where L₁ is attached to an oxygen atom on X, the compound of formula Ia may be selected from the list including, but not limited to:

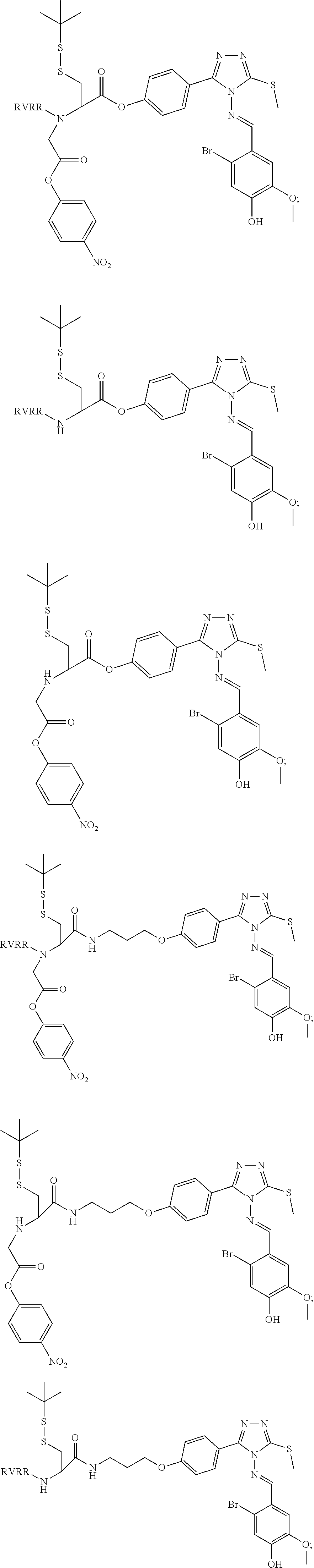

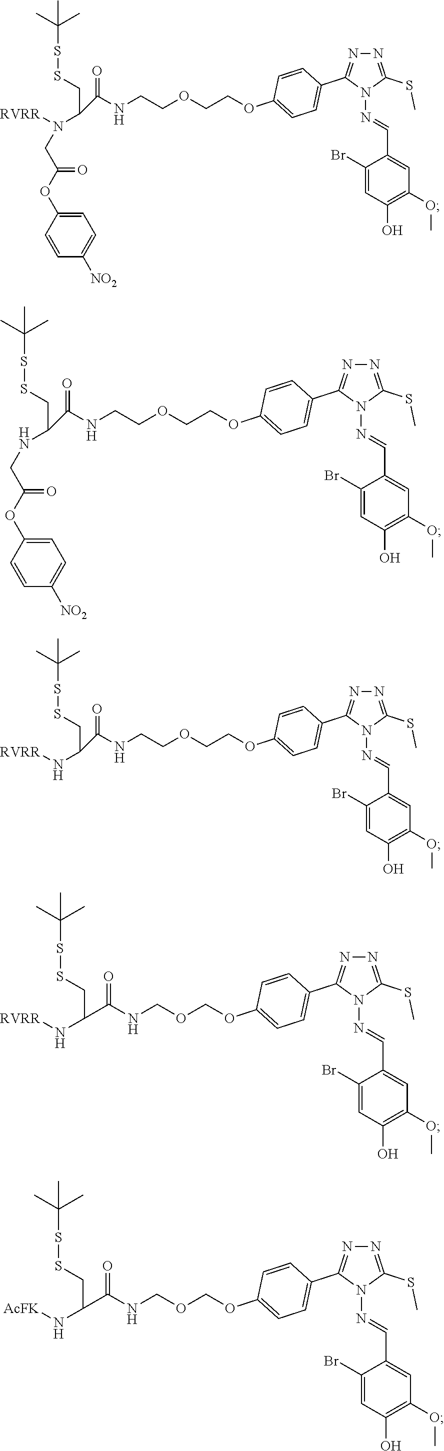

As will be appreciated, the compounds of formula Ia above may also be provided as pharmaceutically acceptable salts or solvates thereof.

As noted hereinbefore, the current invention requires a further component—a compound of formula Ib, or a pharmaceutically acceptable salt or solvate thereof, for its application to generate a desired PROTAC in situ at the desired site of action. Thus, in a second aspect of the invention, there is provided a compound of formula Ib:

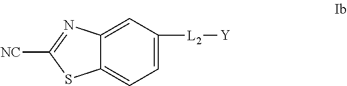

where:
L₂ is a linking group; and
Y is an E3 ligase ligand or a protein of interest ligand,
or a pharmaceutically acceptable salt or solvate thereof.

In embodiments of the invention, L₂ may be attached to Y by way of a nitrogen atom on Y or by way of an oxygen atom on Y.

Thus, in embodiments of the second aspect of the invention where L₂ is attached to a nitrogen atom on Y, it may be selected from:
a bond;

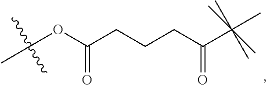

where the crossed line represents the point of attachment to Y and where the wiggly line represents the point of attachment to the rest of the molecule;

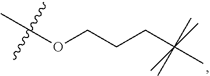

where the wiggly line represents the point of attachment to the rest of the molecule and the crossed line represents the point of attachment to Y;

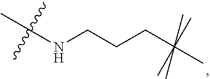

where the wiggly line represents the point of attachment to the rest of the molecule and the crossed line represents the point of attachment to Y;

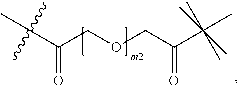

where the wiggly line represents the point of attachment to the rest of the molecule, the crossed line represents the point of attachment to Y and m₂ represents from 1 to 5, such as 1 to 2.

In particular embodiments of the second aspect of the invention where L₂ is attached to a nitrogen atom on Y, Y may be selected from:

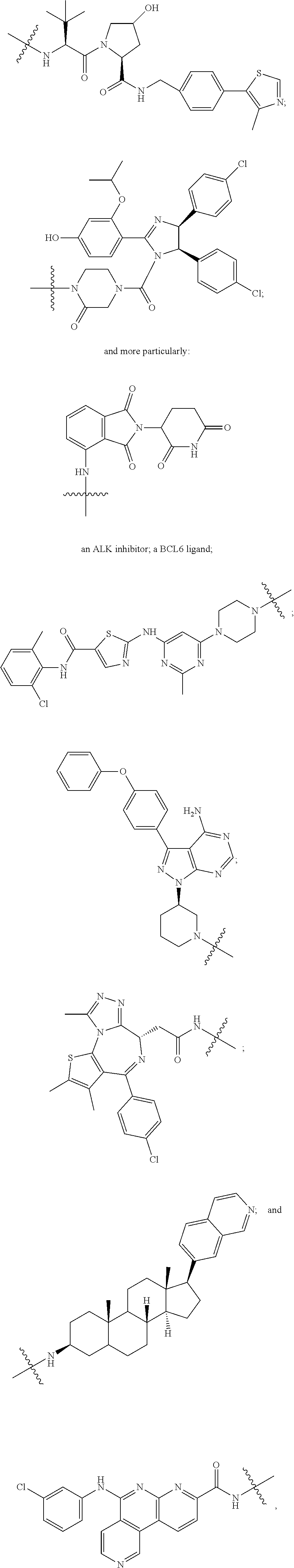

where the wiggly line represents the point of attachment to L₂.

In yet more particular embodiments of the second aspect of the invention where L₂ is attached to a nitrogen atom on Y, Y may be selected from:

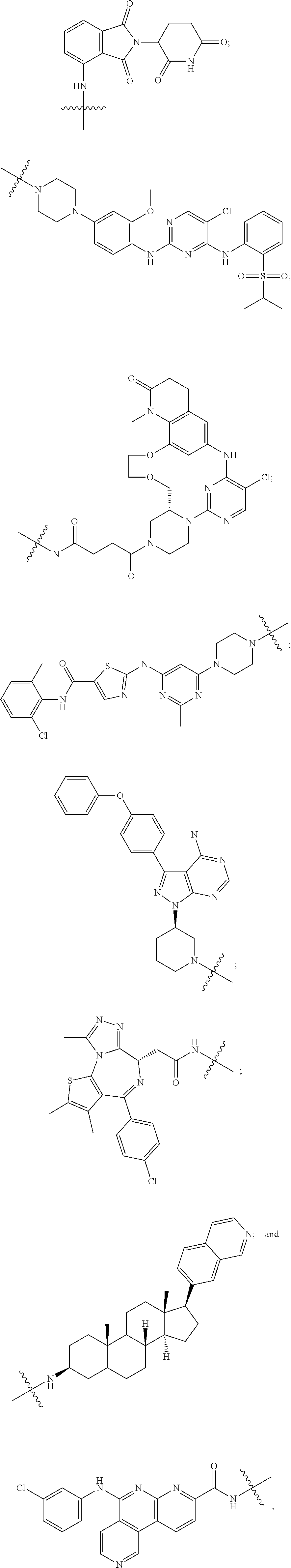

where the wiggly line represents the point of attachment to L₂.

In certain embodiments of the second aspect of the invention where L₂ is attached to a nitrogen atom on Y, the compound of formula Ib may include, but is not limited to, the list selected from:

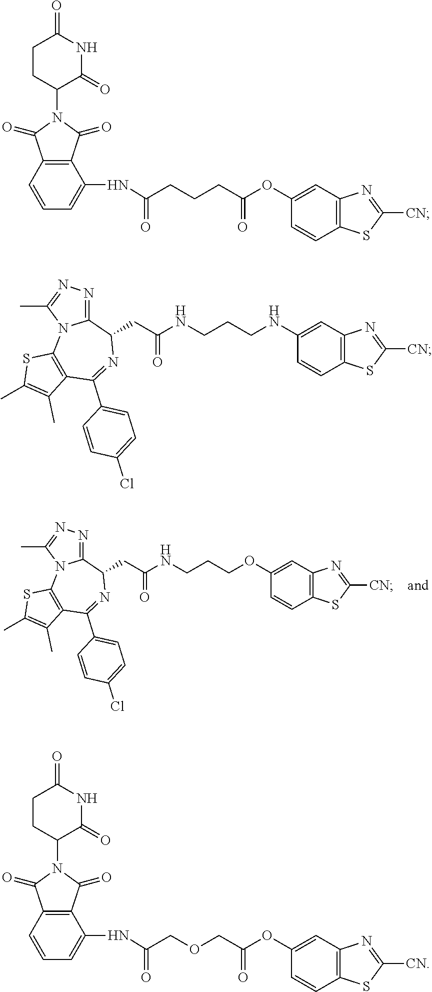 [SMILES: N#Cc1nc2cc(OC(=O)CCCC(=O)Nc3cccc4c3C(=O)N(C3CCC(=O)NC3=O)C4=O)ccc2s1 ; Cc1sc2c(c1C)C(c1ccc(Cl)cc1)=N[C@@H](CC(=O)NCCCNc1ccc3sc(C#N)nc3c1)c1nnc(C)n1-2 ; Cc1sc2c(c1C)C(c1ccc(Cl)cc1)=N[C@@H](CC(=O)NCCCOc1ccc3sc(C#N)nc3c1)c1nnc(C)n1-2 ; "and" ; N#Cc1nc2cc(OC(=O)COCC(=O)Nc3cccc4c3C(=O)N(C3CCC(=O)NC3=O)C4=O)ccc2s1]

As will be appreciated, the compounds of formula Ib above may also be provided as pharmaceutically acceptable salts or solvates thereof.

In alternative embodiments of the invention, L₂ may be attached to an oxygen atom on Y. In such embodiments, Y may be selected from:
a bond; and

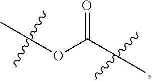 [SMILES: *OC(*)=O]

where the crossed line represents the point of attachment to Y and where the wiggly line represents the point of attachment to the rest of the molecule.

In particular embodiments of the second aspect of the invention where L₂ is attached to an oxygen atom on Y, Y may be selected from:

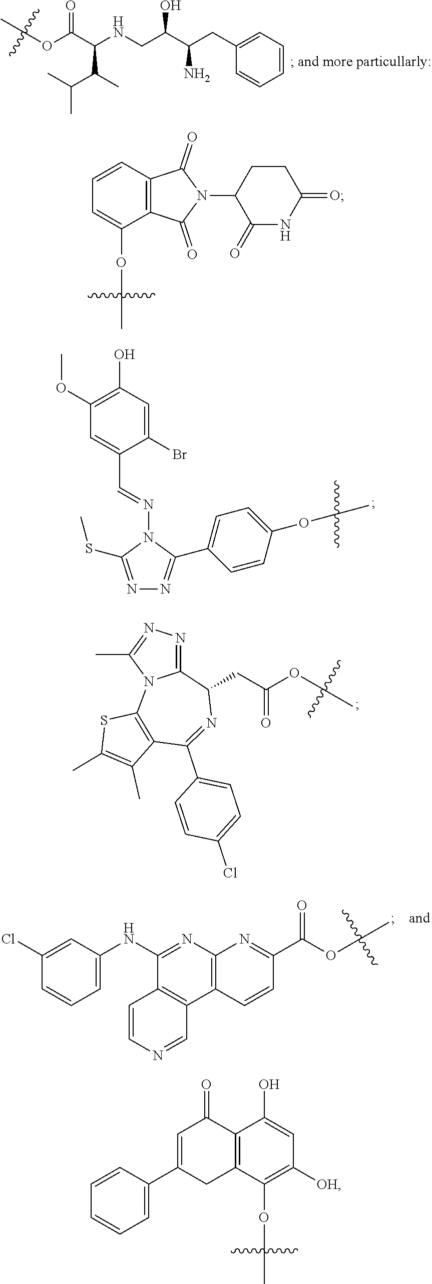

where the wiggly line represents the point of attachment to L₂.

In certain embodiments of the second aspect of the invention where L₂ is attached to an oxygen atom on Y, the compound of formula Ib may include, but is not limited to, the list selected from:

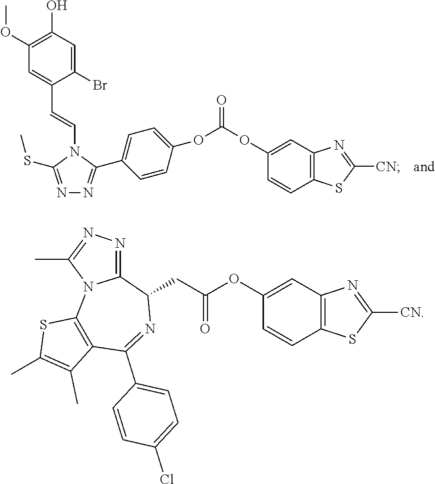 [SMILES: COc1cc(/C=C/n2c(SC)nnc2-c2ccc(OC(=O)Oc3ccc4sc(C#N)nc4c3)cc2)c(Br)cc1O ; "and" ; Cc1sc2c(c1C)C(c1ccc(Cl)cc1)=N[C@@H](CC(=O)Oc1ccc3sc(C#N)nc3c1)c1nnc(C)n1-2]

As will be appreciated, the compounds of formula Ib above may also be provided as pharmaceutically acceptable salts or solvates thereof.

References herein (in any aspect or embodiment of the invention) to compounds of formula Ia and/or Ib includes references to such compounds per se, to tautomers of such compounds, as well as to pharmaceutically acceptable salts or solvates, of such compounds.

Pharmaceutically acceptable salts that may be mentioned include acid addition salts and base addition salts. Such salts may be formed by conventional means, for example by reaction of a free acid or a free base form of a compound of formula Ia and/or Ib with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula Ia and/or Ib in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (+)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula Ia and/or Ib are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

Compounds of formula Ia, as well as pharmaceutically acceptable salts and solvates of such compounds are, for the sake of brevity, hereinafter referred to together as the “compounds of formula Ia”. Compounds of formula Ib, as well as pharmaceutically acceptable salts and solvates of such compounds are, for the sake of brevity, hereinafter referred to together as the “compounds of formula Ib”.

Compounds of formula Ia and/or Ib may contain double bonds and may thus exist as E (entgegen) and Z (zusammen) geometric isomers about each individual double bond. All such isomers and mixtures thereof are included within the scope of the invention.

Compounds of formula Ia and/or Ib may exist as regioisomers and may also exhibit tautomerism. All tautomeric forms and mixtures thereof are included within the scope of the invention.

Compounds of formula Ia and/or Ib may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, while some of the structures disclosed herein may be drawn to disclose a particular stereochemistry, this is only intended to show a particular desired stereochemistry and the structures so drawn may also cover all possible enantiomers, diasteriomers and mixtures thereof (e.g. racemic mixtures thereof). However, in particular embodiments of the invention, the drawn structures may depict preferred stereochemical forms of said structures.

Further embodiments of the invention that may be mentioned include those in which the compound of Ia and/or Ib is isotopically labelled. However, other, particular embodiments of the invention that may be mentioned include those in which the compound of Ia and/or Ib is not isotopically labelled.

The term “isotopically labelled”, when used herein includes references to compounds of Ia and/or Ib in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of Ia and/or Ib that are isotopically enriched at one or more positions in the compound.

The isotopic labelling or enrichment of the compound of Ia and/or Ib may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula Ia and/or Ib is labelled or enriched with a radioactive or nonradioactive isotope, compounds of formula Ia and/or Ib that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

As will be appreciated, the compounds of formula Ia and Ib, or pharmaceutically acceptable salts and solvates thereof, may be presented:
(aai) in two separate pharmaceutical formulations; or
(aaii) in a single pharmaceutical formulation.

Thus, in a further aspect of the invention, there is provided a pharmaceutical formulation comprising a compound of formula Ia, or a pharmaceutically acceptable salt or solvate thereof, as described herein and/or a compound of formula Ib, or a pharmaceutically acceptable salt or solvate thereof, as described herein and a pharmaceutically acceptable carrier, provided that when both of the compounds of formula Ia and formula Ib are present, one of the compounds of formula Ia and formula Ib includes an E3 ligase ligand and the other compound of formula Ia and formula Ib includes a protein of interest ligand.

Compounds of formula Ia and/or Ib may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula Ia and/or Ib will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula Ia and/or Ib in any pharmaceutical formulation used in accordance with the present invention will depend on various factors, such as the severity of the condition to be treated, the particular patient to be treated, as well as the compound(s) which is/are employed. In any event, the amount of compound of formula Ia and/or Ib in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

As will be appreciated, the compounds of formula Ia and Ib (or their pharmaceutically acceptable salts or solvates) are intended to be used in combination to provide the desired efficacy. As such, the compounds of formula Ia and Ib (or their pharmaceutically acceptable salts or solvates) may be provided in a kit of parts. Thus, in a further aspect of the invention, there is provided a kit of parts comprising:
(a) a compound of formula Ia, or a pharmaceutically acceptable salt or solvate thereof, as described herein; and
(b) a compound of formula Ib, or a pharmaceutically acceptable salt or solvate thereof, as described herein. More particularly, the kit of parts may be one in which:
(ai) the compound of formula Ia is provided in a pharmaceutical formulation comprising the compound of formula Ia and a pharmaceutically acceptable carrier; and/or
(aii) the compound of formula Ib is provided in a pharmaceutical formulation comprising the compound of formula Ib and a pharmaceutically acceptable carrier.

As will be appreciated, the compounds of formula Ia and Ib (or their pharmaceutically acceptable salts or solvates) are intended for use in medicine, where they provide their desired effect by combining at the desired site of action to provide a PROTAC molecule with beneficial effect for a subject in need of such treatment.

As such, there is provided:
(AA) a compound of formula Ia, or a pharmaceutically acceptable salt or solvate thereof, as described herein for use as a medicament;
(AB) a compound of formula Ia, or a pharmaceutically acceptable salt or solvate thereof, as described herein for use in the treatment of cancer;
(AC) use of a compound of formula Ia, or a pharmaceutically acceptable salt or solvate thereof, as described herein in the preparation of a medicament to treat cancer; and
(AD) a method of treating cancer, said method comprising the step of providing a subject in need thereof a pharmaceutically effective amount of a compound of formula Ia, or a pharmaceutically acceptable salt or solvate thereof, as described herein.

There is also provided:
(BA) a compound of formula Ib, or a pharmaceutically acceptable salt or solvate thereof, as described herein for use as a medicament;
(BB) a compound of formula Ib, or a pharmaceutically acceptable salt or solvate thereof, as described herein for use in the treatment of cancer;
(BC) use of a compound of formula Ib, or a pharmaceutically acceptable salt or solvate thereof, as described herein in the preparation of a medicament to treat cancer; and
(BD) a method of treating cancer, said method comprising the step of providing a subject in need thereof a pharmaceutically effective amount of a compound of formula Ib, or a pharmaceutically acceptable salt or solvate thereof, as described herein.

Further, there is disclosed:
(CA) a compound of formula Ia, or a pharmaceutically acceptable salt or solvate thereof, as described herein in combination with a compound of formula Ib, or a pharmaceutically acceptable salt or solvate thereof, as described herein for use as a medicament;
(CB) a compound of formula Ia, or a pharmaceutically acceptable salt or solvate thereof, as described herein and a compound of formula Ib, or a pharmaceutically acceptable salt or solvate thereof, as described herein for use in the treatment of cancer;
(CC) a compound of formula Ia, or a pharmaceutically acceptable salt or solvate thereof, as described herein for use in the treatment of cancer wherein the compound of formula Ia is administered sequentially, simultaneously or concomitantly with a compound of formula Ib, or a pharmaceutically acceptable salt or solvate thereof, as described herein;
(CD) a compound of formula Ib, or a pharmaceutically acceptable salt or solvate thereof, as described herein for use in the treatment of cancer wherein the compound of formula Ib is administered sequentially, simultaneously or concomitantly with a compound of formula Ia, or a pharmaceutically acceptable salt or solvate thereof, as described herein;
(CE) use of a compound of formula Ia, or a pharmaceutically acceptable salt or solvate thereof, as described herein in the preparation of a medicament to treat cancer, wherein the compound of formula Ia is administered sequentially, simultaneously or concomitantly with a compound of formula Ib, or a pharmaceutically acceptable salt or solvate thereof, as described herein to a subject;
(CF) use of a compound of formula Ib, or a pharmaceutically acceptable salt or solvate thereof, as described herein in the preparation of a medicament to treat cancer, wherein the compound of formula Ib is administered sequentially, simultaneously or concomitantly with a compound of formula Ia, or a pharmaceutically acceptable salt or solvate thereof, as described herein to a subject;
(CG) a method of treating cancer, said method comprising the step of providing a subject in need thereof a pharmaceutically effective amount of a compound of formula Ia, or a pharmaceutically acceptable salt or solvate thereof, as described herein, wherein the compound of formula Ia is administered sequentially, simultaneously or concomitantly with a pharmaceutically effective amount of a compound of formula Ib, or a pharmaceutically acceptable salt or solvate thereof, as described herein; and
(CH) a method of treating cancer, said method comprising the step of providing a subject in need thereof a pharmaceutically effective amount of a compound of formula Ib, or a pharmaceutically acceptable salt or solvate thereof, as described herein, wherein the compound of formula Ib is administered sequentially, simultaneously or concomitantly with a pharmaceutically effective amount of a compound of formula Ia, or a pharmaceutically acceptable salt or solvate thereof, as described herein.

The term “cancer” will be understood by those skilled in the art to include conditions such as, but not limited to, adrenal cancer, anal cancer, bile duct cancer, bladder cancer, bone cancer, brain tumours, CNS tumours, breast cancer, Castleman disease, cervical cancer, colon cancer, rectum cancer, endometrial cancer, esophagus cancer, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, and Wilms tumour.

Particular cancers that may be mentioned herein may be in the form of solid tumours (such as adrenal cancer, anal cancer, bile duct cancer, bladder cancer, bone cancer, brain tumours, CNS tumours, breast cancer, Castleman disease, cervical cancer, colon cancer, rectum cancer, endometrial cancer, esophagus cancer, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal cancer, hypopharyngeal cancer, liver cancer, lung cancer (e.g. small cell or non-small cell), lung carcinoid tumour, malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, and Wilms tumour).

In particular embodiments that may be mentioned herein, the cancer may be one or more of the following: acute myeloid leukaemia, liver cancer, pancreatic cancer, breast cancer (e.g. triple negative breast cancer) and prostate cancer. In particular embodiments, the PROTAC molecule obtained by the component compounds of formula Ia and formula Ib may be a PROTAC that is targeted for the degradation of:
CDK kinase (e.g. CDK 9, 4/6 etc.) for acute myeloid leukaemia therapy;
TRK for liver cancer;
MDM2 for pancreatic cancer;
BET for triple negative breast cancer;
Estrogen receptor (ER) alpha targeting for Breast cancer; and
androgen receptor (AR) targeting for Prostate cancer.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

When used herein, the term “administered sequentially, simultaneously or concomitantly” includes references to:
administration of separate pharmaceutical formulations (one containing the compound of formula Ia and one containing the compound of formula Ib); and
administration of a single pharmaceutical formulation containing the compound of formula Ia and formula Ib.

The combination product described above provides for the administration of the compound of formula Ia in conjunction with the compound of formula Ib, and may thus be presented as separate formulations, wherein at least one of those formulations comprises the compound of formula Ia and at least one comprises the compound of formula Ib. Alternatively, the product may be presented (i.e. formulated) as a combined preparation (i.e. presented as a single formulation including the compound of formula Ia and the compound of formula Ib).

Depending on the disorder, and the patient, to be treated, as well as the route of administration, compounds of formula Ia and/or Ib may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula Ia and/or a compound of formula Ib.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

Advantages associated with the current invention may include one or more of those listed below.

1. The small molecule EnC-TAC degradation system disclosed herein may display a selective and specific nature to make PROTACs more personalized and simple to use in contrast to traditional inhibitors and PROTAC tools.

2. The PROTAC system disclosed herein is initialized through the involvement of a tumour-specific protease enzyme (e.g. furin, cathepsin B, caspases etc or other enzymes). This solves the concerns associated with tumour selectivity associated with conventional PROTAC systems, in addition to the limited tumour penetration associated with conventional PROTACs.

3. Unlike traditional PROTAC molecules, which are given in large amounts to a subject, and can therefore inhibit ternary complex formation to block degradation, the system disclosed herein can dynamically control the formation of PROTAC at the desired degradation site, thus greatly minimizing the need to provide large quantities of the PROTAC molecule that results in the issue above. Thus, the system disclosed herein may enhance the desired pharmacological performance.

4. There is no need to chemically link the POI recognition ligand together with E3 ligase moiety (e.g. pomalidomide etc) ex vivo, this may greatly simplify the PROTAC synthesis process. Additionally, the new system reduced the concerns associated with limited cell penetration of conventional PROTAC molecules, which have a much higher molecular that the two components disclosed herein, which together form the desired PROTAC molecule at the site of action.

5. Since the PROTAC molecule disclosed herein can be only generated within the localized tumour environment through the specific enzyme reactions, such enzyme controlled fragment conjugation can minimize the hook effect that is encountered in the conventional PROTAC process.

6. Conveniently, the system disclosed herein allows for the POI recognition ligand and the E3 ligase moiety to be modified separately in a highly flexible manner, which can greatly simplify the synthetic efforts and is expected to be of significant benefit for high throughput screening of ligand moieties suitable for PROTAC applications.

7. Moreover, such individual fragments (POI ligand and E3 ligase ligand) with formed with an enzyme responsive peptide sequence or 2-cyanobenzothiazole analog can be swapped between the compound carrying the POI ligand and the compound carrying the E3 ligase ligand. This creates significant linkage flexibility with more options to optimize the structure modification compared to conventionally-integrated PROTACT tools with their structure fixed and higher molecule weight.

8. The enzyme responsive cross-linking of the POI recognition ligand with E3 ligase moiety (e.g. pomalidomide etc) of the current invention may lead to a fluorescent conjugate that provides the opportunity to precisely quantify the overall PROTAC process, and importantly, real-time monitor the proteasome ablation of the tumour signaling pathway, e.g., circadian.

9. Our new method can innovatively expand the usage to other tumour cell systems with their specific proteins, enzymes expression and/or external stimulation responsive reactions, which therefore pave new revenues for targeted PROTAC based tumour therapy in the future.

Further aspects and embodiments of the invention will now be described by reference to the following non-limiting examples.

### EXAMPLES

### Materials

(+)-JQ1 was purchased from MCE. Reagent-grade chemical reagents were purchased from Sigma-Aldrich, Thermo Fisher Scientific, MedChemExpress, and InnoChem. All chemical reactions were performed at ambient condition unless otherwise stated. Thin layer chromatography (TLC) was performed on TLC silica gel 60 F254 glass plates covered with approx. 0.2 mm silica gel. Ultraviolet light was used as the medium of TLC visualization. Radioimmunoprecipitation assay (RIPA) buffer supplemented with protease inhibitor cocktail was purchased from Roche. Phosphatase inhibitor cocktail, primary antibodies (#13440, 1:1000 dilution factor), HIF-1α (#36169, 1:1000 dilution factor) and GADPH (#5174, 1:1000 dilution factor) were purchased from Cell Signalling Technologies. Mini-PROTEAN® TGX™ 4-20% Precast Gels was purchased from Bio-rad. Radiance Q chemiluminescent ECL substrate was purchased from Azure Biosystems. Goat anti-rabbit IgG (H+L) secondary antibody, HRP (#31460, 1:20,000 dilution factor), goat anti-rabbit IgG (H+L) cross-adsorbed secondary antibody, Alexa Fluor 594 (cat #A11012), goat anti-rat IgG (H+L) cross-adsorbed secondary antibody, Alexa Fluor 488 (cat #A11006), rabbit HIF-1α polyclonal antibody (PA1-16601, 1:100 dilution factor), c-Myc (#700648, 1:1000 dilution factor), PARP1 (#MA5-15031, 1:500 dilution factor), VEGF (#P802, 1:500 dilution factor), dual staining molecular probe containing Annexin V (AnnV) and PI (#88-8005-74), polyvinylidene difluoride (PVDF) membrane (Product #: 88518), O.C.T. compound and CA-IX (#MA5-29076, 1:1000 dilution factor) were purchased from ThermoFisher Scientific, Waltham, MA, USA. BRD4 (ab243862, 1:1000 dilution factor), Ki67 (cat #ab15580), B-Tubulin (ab6046, 1:10,000 dilution factor), Alexa Fluor® 488-goat anti-rabbit IgG secondary antibody (ab150077, 1:1000 dilution factor), and Alexa Fluor® 594 Anti-α Tubulin antibody [DM1A]—Microtubule Marker (ab195889, 1:200 dilution factor) were purchased from Abcam, Cambridge, UK. Dulbecco's modified Eagle's medium (DMEM), fetal bovine serum (FBS), and I-glutamine were purchased from Gibco, USA. Streptomycin was purchased from Nacalai Tesque, Kyoto, Japan. CD31 (cat #553370) was purchased from BD Biosciences, Franklin Lakes, NJ, USA. ARV-825 (#S8297) was purchased from Selleckchem. Tris-buffered saline with 0.1% Tween® 20 detergent (TBS-T), bovine serum albumin (BSA, Product #:A9418), paraformaldehyde (Product #: 158127), penicillin (CAS: 87-08-1), 1% Tween 20, 3% Triton X (CAS: 9036-19-5), horse serum (Product #: H1138), DAPI (CAS: 28718-90-3), PBS (Product #: 806552), sucrose (Product #: S0389), Bortezomib (Product #: 68193), Hoechst 33258 (Product #: 94403), β-tubulin antibody (Product #: T8328), resorufin dye (Product #: 73144), TOX-8 medium solution (EC #: 263-718-5), and Mowiol (Product #: 81381) were purchased from Sigma-Aldrich.

Cells: Hela cells, human embryonic kidney Hek293T cells, human breast cancer MDA-MB-231, mouse breast cancer 4T1 cells, U-20S cell line (#HTB-96), and mouse melanoma cell line B16F1 were purchased from American Type Culture Collection (ATCC), Manassas, VA, USA.

### Analytical Techniques

### Mass Spectroscopy (MS)

Mass spectra were measured on a ThermoFinnigan LCQ Fleet MS instrument and a ThermoFinnigan LCQ Deca XP MAX instrument for electrospray ionization (ESI) measurements.

Nuclear magnetic resonance (NMR) spectroscopy Proton NMR (1H-NMR) and proton-decoupled carbon-13 NMR (¹³C{1H}-NMR) spectra were measured on a Bruker Avance III 400 (BBFO 400) Ultrashield Plus 400 MHz magnet with auto-tunable BBFO probe (5 mm) or on a Bruker Avance III HD 600 MHZ (14.1 T) wide-bore NMR spectrometer.

Spectra were recorded in accordance with the downfield ppm of tetramethylsilane. Calibration was performed with reference to the deuterium NMR solvent (CD₃OD: 3.31 [MeOD], DMSO-d₆: 2.50 [dimethyl sulphoxide]) and carbon NMR solvent (CD₃OD: 49.00 [MeOD], DMSO-d₆: 39.52 [dimethyl sulphoxide]). MestReNova (v14.0) was used to process all the molecular NMR analysis. The relative number of protons were integrated, and the coupling constants were unified as Hertz (Hz). Chemical shifts (δ) of molecular structure were reported, multiplicity (s, singlet; d, doublet; t, triplet; q, quartet; p, pentet; m, multiplet).

### LC-MS

Structural mass analysis was performed by ThermoFinnigan LCQ Deca XP MAX Mass Spectrometer System.

### General Procedure for Preparative Reversed-Phase High-Performance Liquid Chromatography (RP-HPLC) Purification

Compounds were purified over HPLC (Shimadzu) system using RP-HPLC Alltima C-18 column of 250×10 mm at a flow rate of 3 mL min-1. The solvent system used was 30-100%, v/v, MeCN/H₂O. The purified compounds in H₂O were solidified in N₂ liquid and freeze-dried in Labconco™ FreeZone™ 2.5 L Freeze Dryer.

### General Procedure for Cell Culture

Adherent cells were cultured in TPP tissue culture flask 25 cm² at 37° C. and 5% CO₂. HeLa cells, human embryonic kidney Hek293T cells, human breast cancer MDA-MB-231, mouse breast cancer 4T1 cells, mouse melanoma cell line B16F1, and U-20S cells were cultured in DMEM supplemented with 10% FBS and 1% penicillin/streptomycin at 37° C. and 5% CO₂ in a humidified atmosphere. All cells were routinely split 1-2 times per week when 90% confluence was attained, and were not used beyond passage 30.

### Example 1. Synthesis of PROTAC T44 with Longer Distance Between E3 Ligase Ligand (Pomalidomide) and CK2α Recognition Moiety (GO289) Based on Conventional PROTAC Strategy

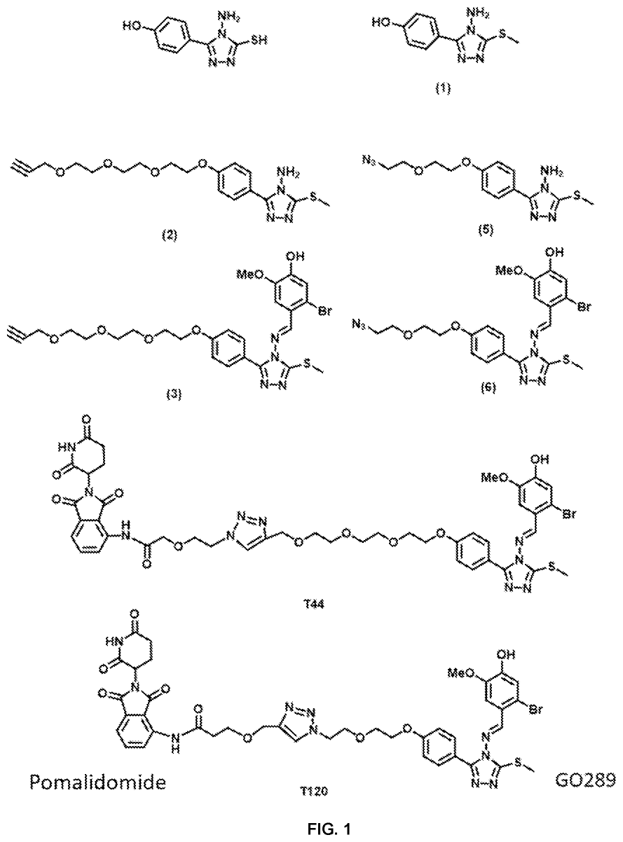

The degradation molecules were developed by optimizing the proximity of E3 ligase moiety (e.g. pomalidomide, and etc.) and recognition ligand (e.g. GO289 for CK2α, and etc.) to examine the possibility of effective decomposition of circadian protein of CK2α (FIG. 1).

### Synthesis of T44

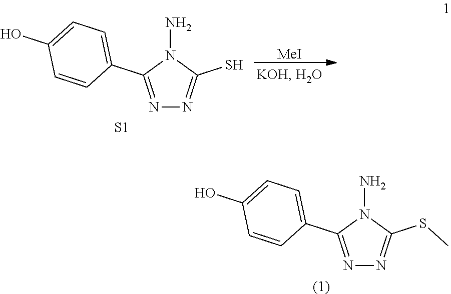

To a suspension of 4-amino-5-(4-hydroxyphenyl)-2,4-dihydro-3H-pyrazole-3-thione (S1, 500 mg, 2.4 mmol) in H₂O (20 mL), potassium hydroxide (148.1 mg, 2.6 mmol, 1.1 equiv) and iodomethane (177.25 mg, 2.6 mmol, 1.1 equiv) were added at room temperature (r.t.). After stirring the mixture at r.t. for 14 h, the precipitated solid was filtrated and washed with H₂O to afford 1 (492.4 mg, 92% yield) as a yellow solid.

¹H NMR (DMSO-d₆, 400 MHZ) δ 8.01-7.95 (m, 2H), 7.54-7.46 (m, 2H), 6.13 (s, 2H), 2.61 (s, 3H); ¹³C NMR (DMSO-d₆, 100 MHZ) δ 154.5, 154.1, 129.5, 128.5, 127.7, 126.9, 13.7; MS (ESI) m/z calcd for C₁₀H₁₃N₃OS [M+H]⁺: 223.06 found 223.11.

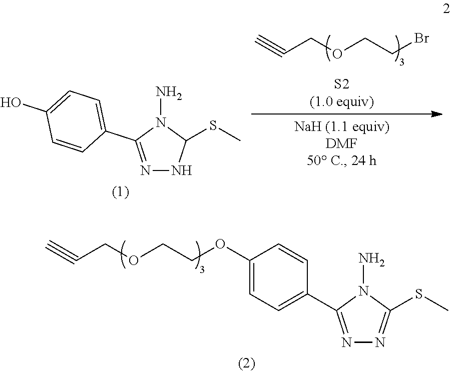

To a solution of 1 (100 mg, 0.45 mmol) in dimethylformide (DMF), sodium hydride (NaH, 11.9 mg, 0.49 mmol) was slowly added at 0° C. After stirring at 30 min at r.t., a solution of propargyl-PEG₃-bromide (S2, 113 mg, 0.45 mmol) in DMF (0.5 mL) was added to the mixture at 0° C. The reaction was stirred at 60° C. for 24 h before being quenched with saturated aqueous NH₄Cl. After that, the mixture was extracted with EtOAc, washed with brine, dried with Na₂SO₄, and the solvent was removed in vacuo. The product was then purified by column chromatography with MeOH/DCM (1/20) to obtain a pale-yellow oil compound 2 (56% yield).

¹H NMR (300 MHZ, MeOD) δ 6.40 (t, J=8.8 Hz, 2H), 5.55 (d, J=8.8 Hz, 2H), 2.67 (dd, J=8.9, 3.4 Hz, 4H), 2.38-2.31 (m, 2H), 2.19 (dd, J=3.7, 1.8 Hz, 2H), 2.15 (d, J=3.6 Hz, 6H), 1.81 (d, J=11.6 Hz, 1H), 1.30 (dd, J=13.2, 10.9 Hz, 1H), 1.16 (d, J=9.1 Hz, 3H). MS (ESI) m/z calcd for C₁₈H₂₄N₄O₄S [M+H]⁺: 393.15 found 393.31. MS (ESI) m/z calcd for C₁₈H₂₄N₄O₄S [M+H]⁺: 393.15 found 393.31.

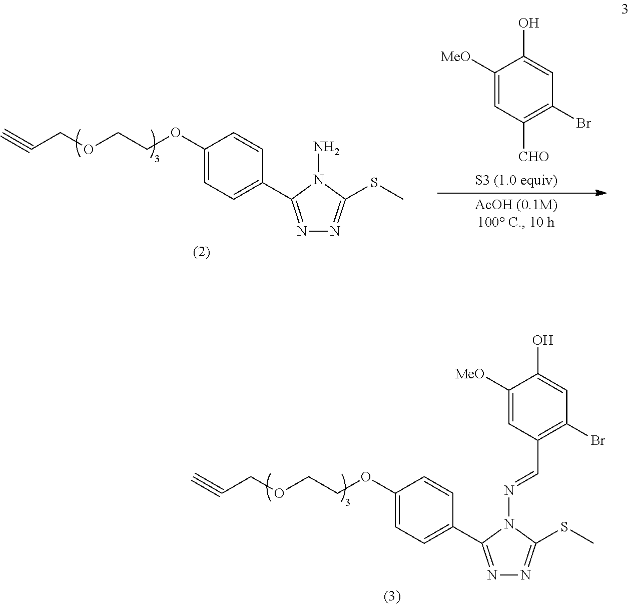

To a solution of 2 (100 mg, 0.26 mmol) and 2-bromo-4-hydroxy-5-methoxybenzaldehyde (S3, 60.6 mg, 0.26 mmol) in AcOH was heated at 100° C. for 10 h. After removal of the solvent, the mixture was purified by column chromatography with MeOH/DCM (1/40). 3 was obtained as a pale yellow viscous oil (73% yield).

MS (ESI) m/z calcd for C₂₆H₂₉BrN₄O₆S [M+H]⁺: 606.10 found 607.28.

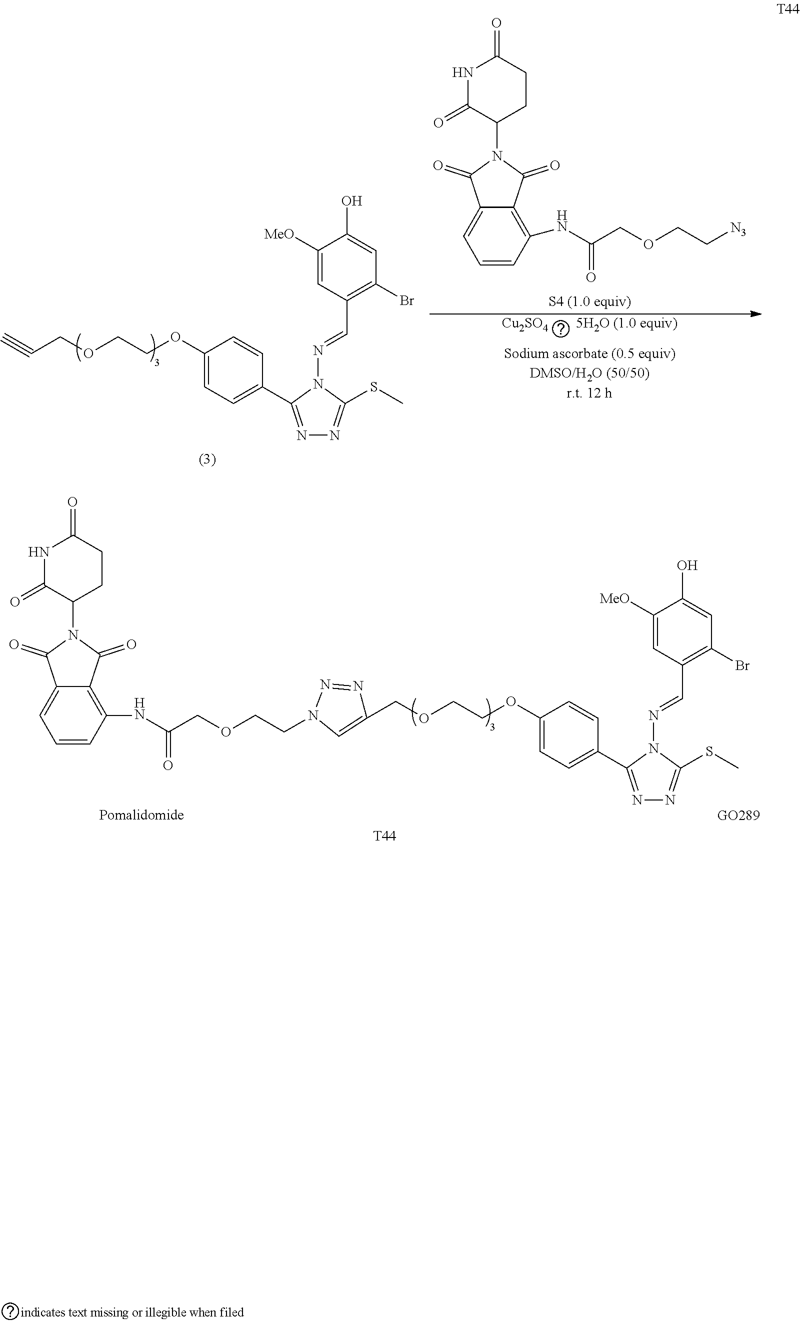

To a solution of 3 (10 mg) in a mixture of DMSO/H₂O (50/50), Cu₂SO₄·5H₂O (4.3 mg, 1.0 equiv.), sodium ascorbate (1.7 mg, 0.5 equiv.), and pomalidomide-PEG₁-azide (S4, 6.4 mg, 1.0 equiv) were added. The reaction mixture was stirred under r.t. for 12 h. The crude product was extracted with brine and DCM before being purified by RP-HPLC to obtain a pale yellow oil pure product T44 (91% yield).

MS (ESI) m/z calcd for C₄₃H₄₅BrN₁₀O₁₂S [M+H]⁺: 1005.21 found 1007.29.

### Example 2. Synthesis of PROTAC T120 with Shorter Distance Between E3 Ligase Ligand (Pomalidomide) and CK2α Recognition Moiety (GO289)

### Synthesis of T120

2 (100 mg, 0.26 mmol) was added to a solution of bromo-PEG1-azide (1.0 equiv) in DMF (0.5 mL) at 0° C. by following the protocol in Example 1. The product was taken for the synthesis of the PROTAC molecule T120 with shorter chain by following the protocol in Example 1. The final product was harvested and worked up by RP-HPLC to obtain a pale-yellow oil compound T120 (40% yield).

¹H NMR (500 MHZ, MeOD) δ 7.28 (s, 1H), 7.06 (d, J=8.5 Hz, 1H), 6.41 (s, 1H), 6.18 (s, 1H), 6.17 (s, 1H), 6.13-6.08 (m, 1H), 6.07 (s, 1H), 5.93 (d, J=7.2 Hz, 1H), 5.47 (s, 1H), 5.46 (s, 1H), 5.45 (s, 1H), 3.51 (dd, J=12.7, 5.4 Hz, 1H), 3.06 (s, 2H), 3.01 (d, J=5.0 Hz, 2H), 2.55-2.50 (m, 2H), 2.37-2.34 (m, 2H), 2.33 (s, 3H), 2.23-2.20 (m, 2H), 2.18 (d, J=5.7 Hz, 2H), 1.27 (d, J=13.9 Hz, 1H), 1.20 (s, 1H), 1.16 (s, 1H), 1.16 (s, 3H), 1.12 (d, J=13.1 Hz, 1H), 1.04 (t, J=5.7 Hz, 2H), 0.59 (s, 1H). MS (ESI) m/z calcd for C₄₀H₃₉BrN₁₀O₁₀S [M+H]⁺: 931.18, found 931.37.

### Example 3. Immunofluorescence Staining of T44 and T120

After the synthesis of T44 and T120 in Examples 1 and 2, their properties for protein degradation were examined. Hek293T, MCF-7, and U205 cells were chosen as they can express different levels of CK2α protein.

### Immunofluorescence Staining

Upon culture under 37° C. for 24 h, the cells were incubated with the PROTAC molecules T120 (0.1 μM or 1 μM) and T44 (0.1 μM or 1 μM) for a prolonged time duration (0-30 h). Upon washing with PBS buffer thrice, the protein degradation was examined by immunofluorescence staining.

Each group of cells with density of 5×10⁴ cells in each well of 8-well ibidi-dishes was treated with T120 or T44 at different concentration (0.1 μM or 1 μM) and time (0 h, 6 h, 18 h or 30 h). After treatment, the cells were fixed with 4% paraformaldehyde for 15 min, permeabilized with 0.25% Triton™ X-100 for 10 min and blocked with 5% BSA for 1 h at room temperature. The cells were then labelled with CK2α Rabbit polyclonal Antibody (Cell signalling, #2656) in 1% BSA and incubated for 3 h at r.t. Subsequently, Alexa Fluor 488-Goat Anti-Rabbit IgG secondary antibody was stained for 30 min at r.t. The nucleus was stained with Hoechst 33342 for 30 min at r.t. The degradation of CK2α protein was visualized with Carl Zeiss LSM 800 microscopes. (Hoechst 33342: Ex=405 nm, Em=460/50 nm; Alexa Fluor 488: Ex=488 nm, Em=515/30 nm).

### Results and Discussion

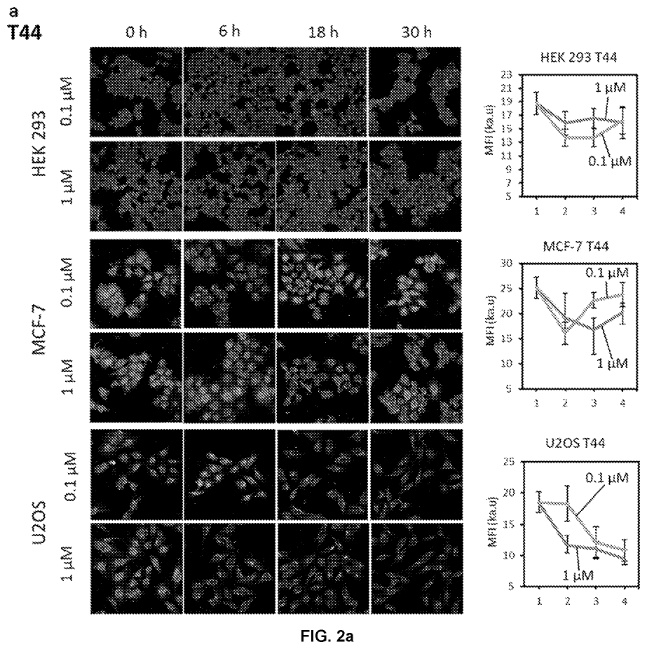

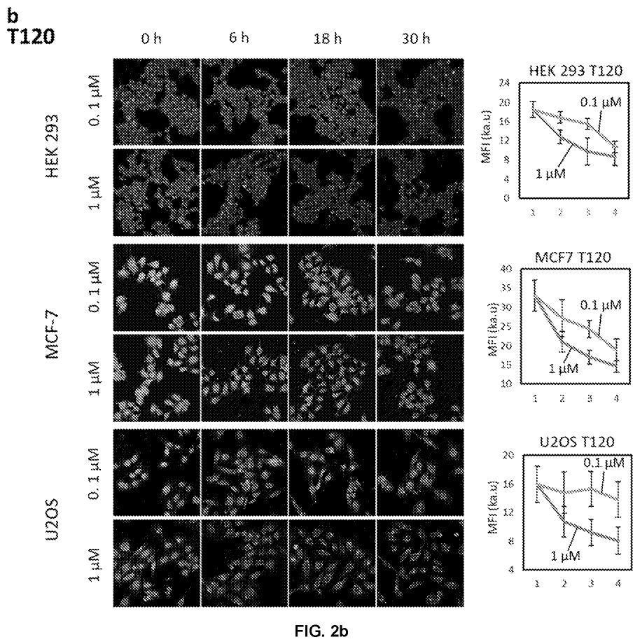

As shown in (FIG. 2), upon incubation of PROTAC molecules (T44 and T120) with the cells, and subsequent fluorescence staining by the Alexa Fluor 488-labelled CK2α antibodies, decreased fluorescence signals were observed in the cells especially with prolonged time duration (up to 20 h), suggesting the degradation effect initialized by the PROTAC molecules. Compared to T44, T120 demonstrated a greater decrease in fluorescence, clearly demonstrating the better effect of protein degradation by T120. The distance between the E3 ligase ligand, pomalidomide, and CK2α recognition moiety (GO289) will be critical for targeted degradation, and the close proximity between pomalidomide, and GO289 will be beneficial for CK2α degradation.

### Example 4. Concentration-Dependent Test for CK2α Degradation

Concentration-dependent test was carried out to investigate the effect of T44 and T120 on different protein degradation.

### Sodium Dodecyl Sulfate-Polyacrylamide Gel Electrophoresis (SDS-PAGE)

Based on the established method (From Merck), the lysates were loaded into the wells in a Mini-PROTEAN® TGX™ 4-20% Precast Gel and run for separation in 30 min under 200 V.

### Concentration-Dependent Test

Cell lines including HEK293T and MCF-7 was incubated with varied concentrations of T44 or T120 for 24 h. After that, the protein level was evaluated by the above immunofluorescence staining protocol. Alternatively, the protein level was evaluated by Western blot analysis. Particularly, The lysates of each group of cells were collected and centrifuged at 12,000×g for 20 min at 4° C. Protein levels in supernatants were determined using Nanodrop and equalized to the same concentration and boiled for 10 min with SDS-PAGE sample loading buffer before being separated using SDS-PAGE and transferred to the PVDF membrane. The membrane was then blocked with 5% BSA-TBST blocking buffer overnight at 4° C. Subsequently, CK2α Rabbit polyclonal Antibody (Cell signalling, #2656) was incubated overnight at 4° C. in 1% BSA-TBST buffer. After series of washing, the goat-anti rabbit IGG (H&L) secondary antibody was added and incubated for 1 h in 1% BSA-TBST. All signals were visualized using AMERSHAM (Image quant 800) system.

### Results and Discussion

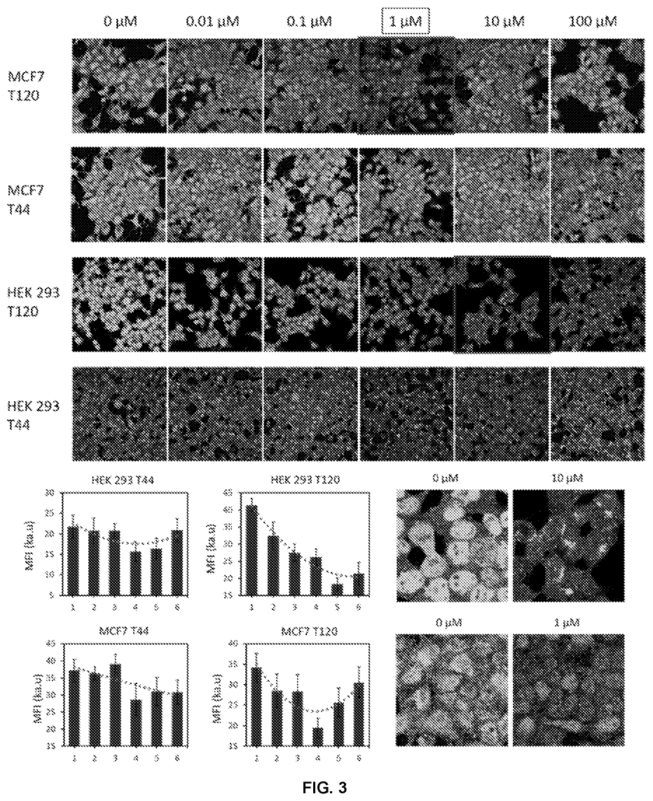

As shown in FIG. 3, the degradation effect is cell-dependent for T44, and there was no obvious fluorescence change upon the incubation of different concentrations of T44 with MCF-7 cells, indicating the weak protein degradation in these cells. In contrast, similar treatment with T120 led to good concentration-dependent degradation effect to decompose CK2α. Although live cell treatment with higher concentration of T120 showed enhanced fluorescence decrease, continuous increase in the concentration of T120 may not always result in better degradation effect. The cellular treatment with much higher concentration (>1 μM in MCF-7 cells and >10 μM in Hek293T cells) caused less effect on CK2α decomposition, suggesting that the molecule crowding in the protein recognition would be one important factor that contributes to protein degradation.

### Example 5. First-In-Class and ENCTACs Based PROTAC Strategy

Inspired by the degradation of CK2α in live cells in Example 4, we developed our first-in-class and personalized PROTAC strategy to achieve selective and specific circadian protein degradation in live cells. The traditional approach in PROTAC degradation works fine but it poses a technical challenge. For instance, the lack of diseases targeting and selectivity (e.g. for cancers, and etc.), limited understanding of quantitative degradation in living system, incapability in pharmacological monitoring of PROTAC process in real-time, tedious synthesis of linking conjugates for ubiquitinated recruitment, and less cellular uptake of high molecular weight of PROTAC would be the concerns that may hamper its further applications in effective cancer therapy. Therefore, we developed a new and personalized PROTAC strategy for efficient protein degradation that meets the demanding challenge encountered in the field.

Instead of using whole molecules with two functional parts, E3 ligase ligand (pomalidomide) and CK2α recognition moiety (GO289) that have to be integrated into one whole structure before their reaction for PROTAC function, the POI recognizer and E3 ligase recruiter can be simply modified with specific tumour responsive moieties (e.g. by tumor enzyme responsive small molecules, peptide sequence, and etc.) and 2-cyanobenzothiazole, respectively, in our new design. Upon effective cell penetration of the two modified fragments into the tumour cells, the tumour specific environment reaction (e.g. acidic pH, radical, light excitation, different enzyme reactions including protease and other enzymes, and etc.) could lead to the cleavage of the recognition sequence that orthogonally induce covalent cross-linking between the exposed cysteine and CBT, thus achieving intrinsic PROTAC trapping within the tumour environment for targeted degradation of POI (FIG. 4). Therefore, our new strategy can realize selective protein degradation in tumour areas with no need for conjugation of affinity ligands. In addition, the overall process will not involve chemical linkers that are traditionally designed and utilized to connect degraded POI with E3 ligase moiety, thus minimizing the concerns of tedious synthesis and limited cell penetration suffered due to larger molecule weight of the PROTAC tools. More importantly, the enzyme responsive cross-linking product demonstrates fluorescence property, which therefore provides great opportunity to precisely quantify the PROTAC and visualize in real-time the proteasome ablation of cancer POI.

As a proof-of-concept, we utilized the selective and tumour specific ENCTAC PROTAC tool to evaluate the therapeutics effects of circadian perturbations small molecules in a cancer cell model via their specific degradation of CK2α which is one important kinase in post-translational regulation. This ubiquitous kinase possesses key functions in cell growth and pathogenesis of cancer development. Moreover, unlike the counterpart of kinase CK1, the role of CK2α in circadian clock machinery has not been well characterized yet. Therefore, from here, the tagged proteins of CK2α will be exploited for their tumour protease enzyme (e.g. furin, CtsB, caspases, and etc.) regulated covalent linking with ubiquitinated E3 ligase, which results in tumour specific circadian pathway degradation, and thus realize drug-free tumour therapy in living system. Such a unique and innovative platform, termed as tumour specific PROTAC, not only demonstrates a promising concept of tumour targeting PROTAC therapy, but also provides the opportunities to innovatively expand the usage to other tumour enzymes, which therefore pave new revenues for personalized tumour therapy in the future.

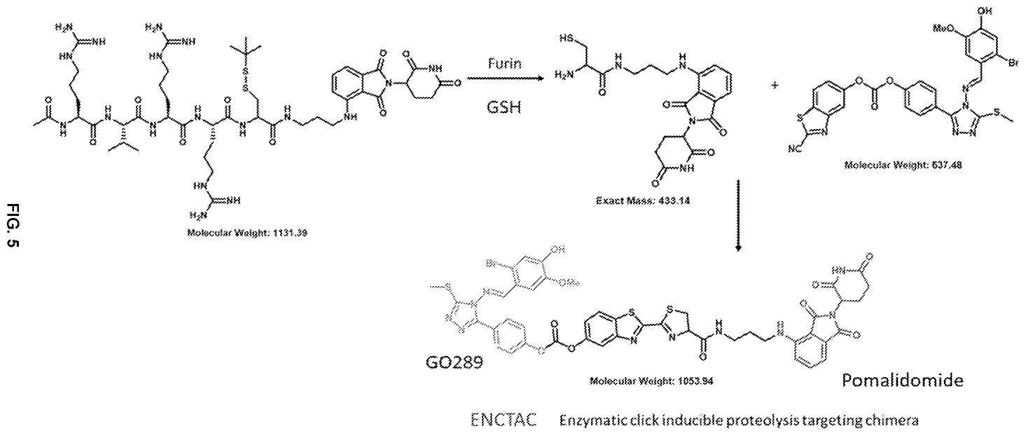

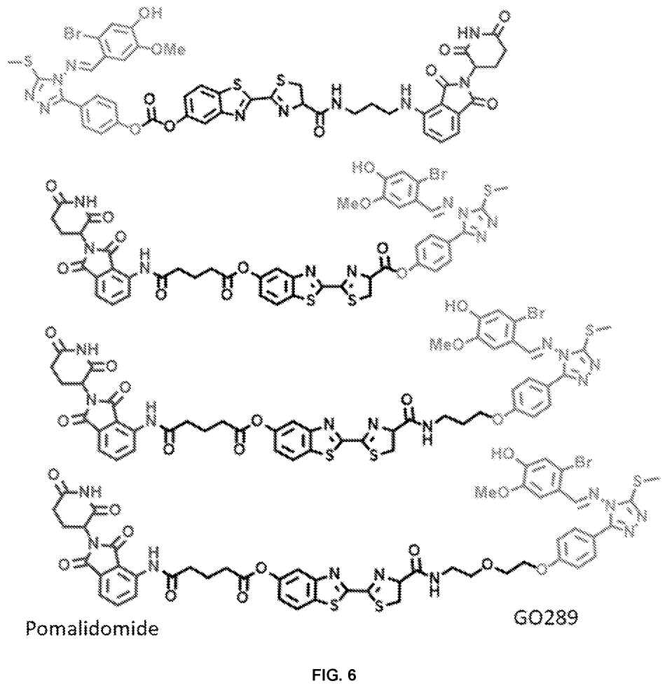

FIG. 5 demonstrates the synthetic routes for our new enzyme responsive peptide PROTAC moieties. To optimize the distance to facilitate enzyme reaction and ubiquitine-mediated target protein degradation, different PEG linkers can be used to modulate the distance between pomalidomide and GO289 moiety, as shown in FIG. 6.

### Example 6. Furin Enzyme Reaction to Form ENCTAC Moieties

With reference to the proof-of-concept in FIG. 3, the furin protease responsive peptide sequence was chosen to conjugate with pomalidomide, while another POI, CK2α protein ligand (GO289 moiety), will be modified with 2-cyanobenzothiazole analog. The reason furin was chosen is mainly attributed to their properties as a membrane-localized proteolytic processing enzyme that is ubiquitously expressed and functions within secretory and endocytic pathways. Furin is also known to be involved in the intramembrane processing of several kinds of matrix metalloproteinases (MMPs), which were found to have elevated levels in several types of human cancers. Moreover, furin enzymes also bear the activities contributing to chronic pathological conditions, maturation of bacterial toxins, and propagation of many non-enveloped or lipid-enveloped viral pathogens, which are prerequisite processes to mediate bacterial or viral invasion (including COVID-19 virus, and others) into host cells.

Typically, the furin responsive sequence (RVRRC) overexpressed in tumours will be flanked with pomalidomide (FIG. 5). Upon the specific furin enzyme reaction, the peptide sequence will be cleaved with cysteine exposed as the terminal. The cysteine can subsequently bio-orthogonally conjugate with CBT analog-modified GO289, thus specifically triggering the formation of PROTAC at the spot of tumour site that achieves the expected protein degradation.

As one membrane-localized proteolytic enzyme, furin belongs to the pro-protein convertase (PC) family and is known to be critical to tumor progression, angiogenesis and metastasis. Recent studies demonstrated that furin can be used for real-time imaging and regulating of tumor cell activity. Here, we designed and synthesized a furin responsive peptide sequence that would be modified with a CK2α ligand (GO289).

Typically, the furin peptide sequence (RVRRC) is prepared through solid phase synthesis, and is subsequently conjugated with GO289. If poor biocompatibility or potential steric hindrance is encountered in the furin reaction, the oligo-(PEG)ₙ linker will be introduced between RVRRC and GO289 precursor. After chemical synthesis, the crude product will be purified by RP-HPLC, and the final products will be further characterized by NMR and MS analysis.

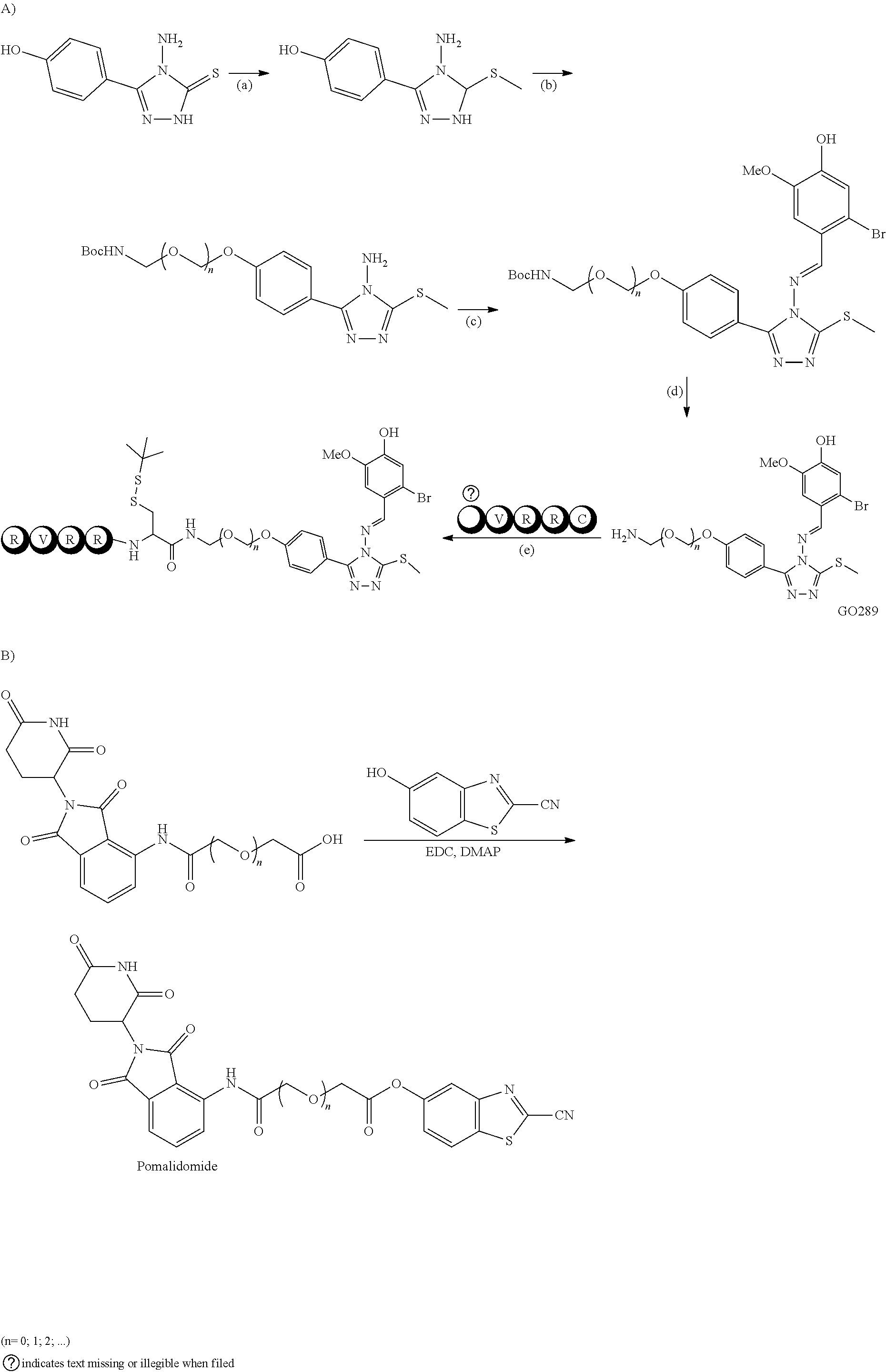

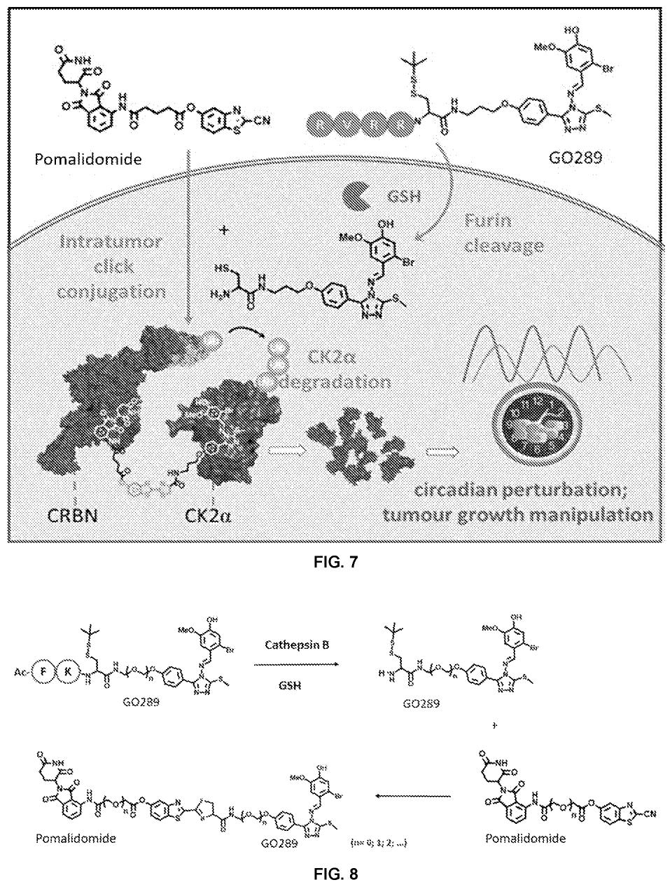

In our system, upon furin enzymatic activation, the tumour specific furin protease enzyme hydrolysis can trigger peptide cleavage, and further orthogonally cause covalent cross-linking conjugation between the exposed cysteine and CBT fragments that have been individually modified with CK2α kinase recognizer GO289 and pomalidomide, a commonly used E3 ligase CRBN recruiting moiety, therefore selectively localizing the PROTAC (FIG. 7) within the tumour environment for targeted degradation of CK2α circadian kinase regulator.

### Example 7. CtsB Protease Enzyme Reaction to Form ENCTAC Moieties

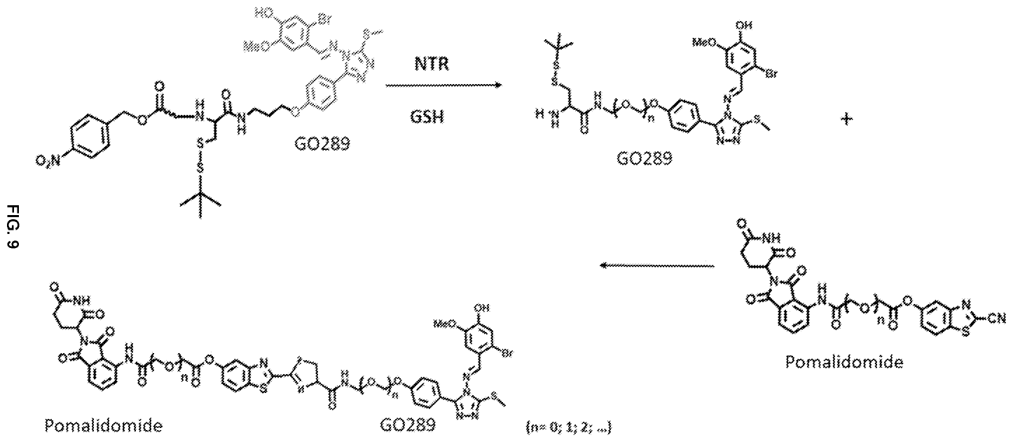

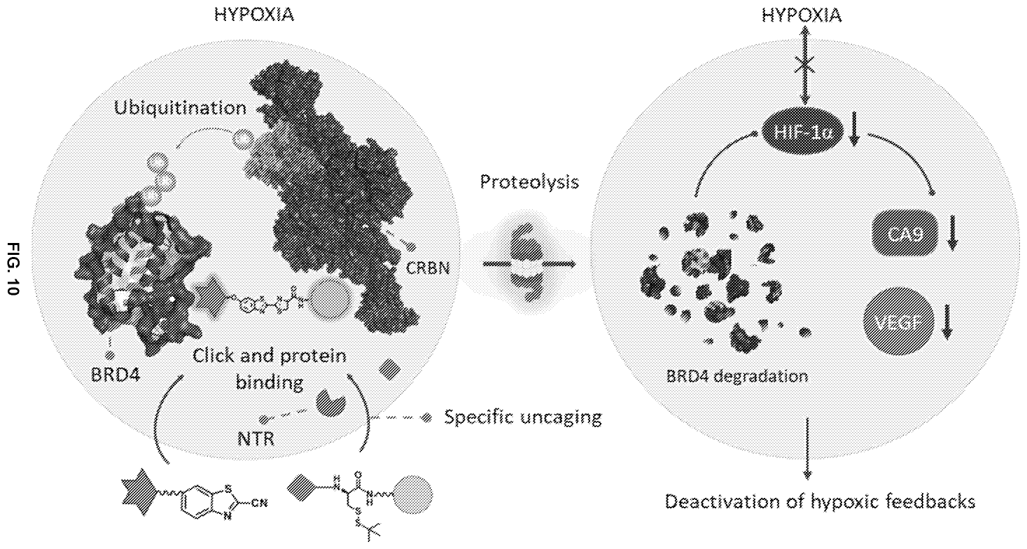

Another tumour specific enzyme was used to achieve localized PROTAC degradation of CK2α kinase degradation. In this example, CtsB enzyme which is one important lysosomal cysteine protease overexpressed in various malignant tumours to process intracellular protein degradation and regulate cancer pathology, was used to process the tumour specific ENCTAC moieties (FIG. 8).

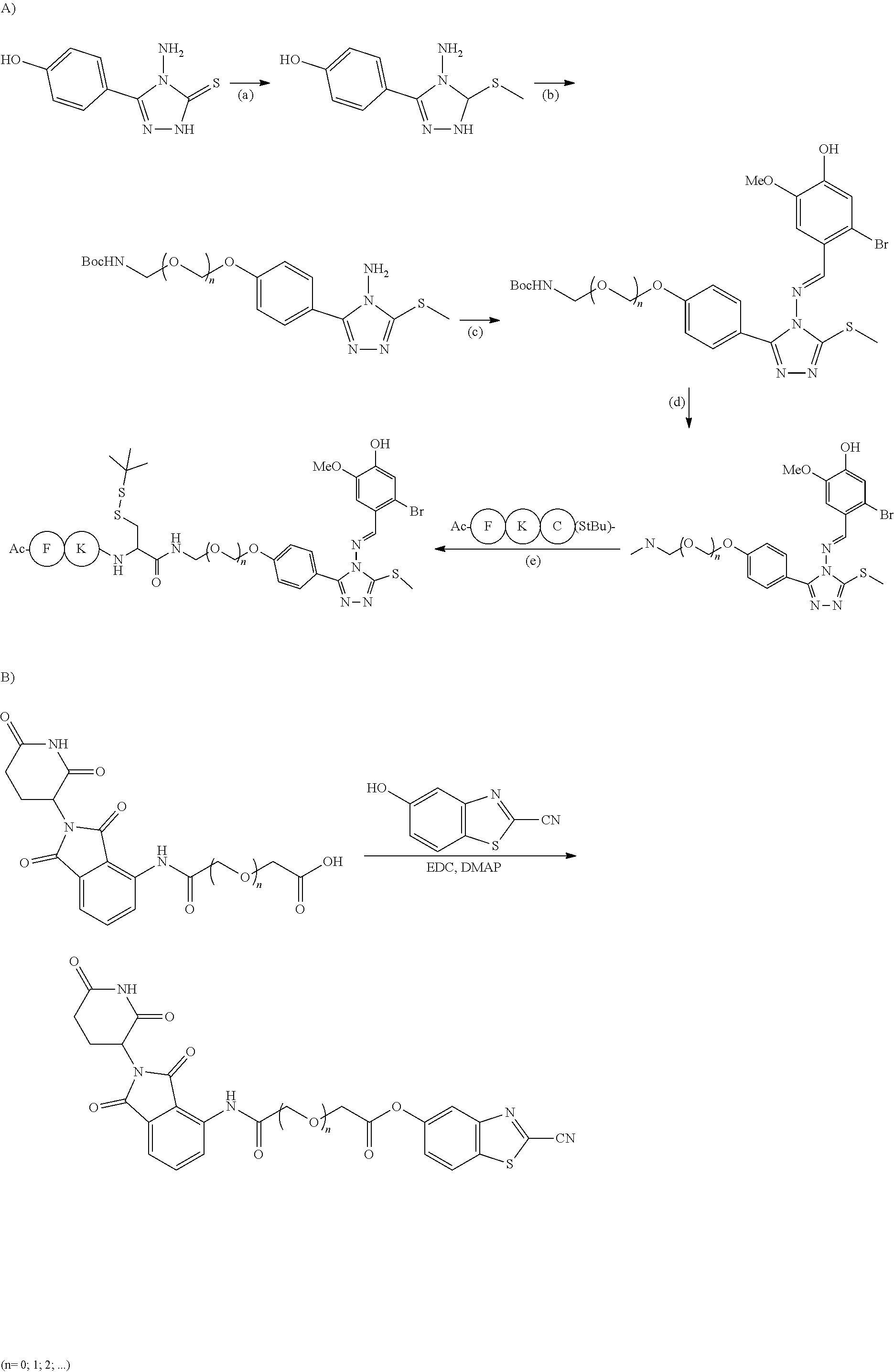

### Example 8. Hypoxia NTR Enzyme Reaction to Form ENCTAC Moieties

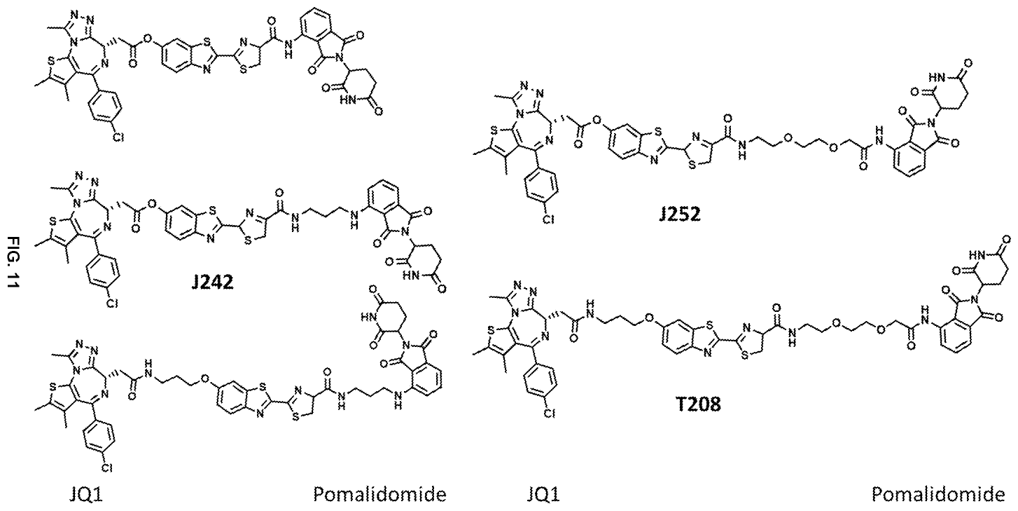

The specifically controlled ENCTAC moieties into tumour can be also achieved through NTR enzyme which is one typical evolutionarily related protein usually involved in the reduction of nitrogen-containing compounds such as those with nitro functional groups, to regulate reduction and maintain the functions in hypoxia conditions. So far, most solid tumours are known to feature hypoxia properties, which can cause an imbalance between oxygen consumption and supply. Moreover, NTR are known to overexpress in most of the hypoxia solid tumours, and has been commonly used as a target for new drug discovery and theranostic applications. Here, NTR enzyme was used to achieve localized PROTAC degradation of CK2α kinase degradation, and to form the tumour specific ENCTAC moieties (FIG. 9).

### Synthesis of PA-JW-GO289

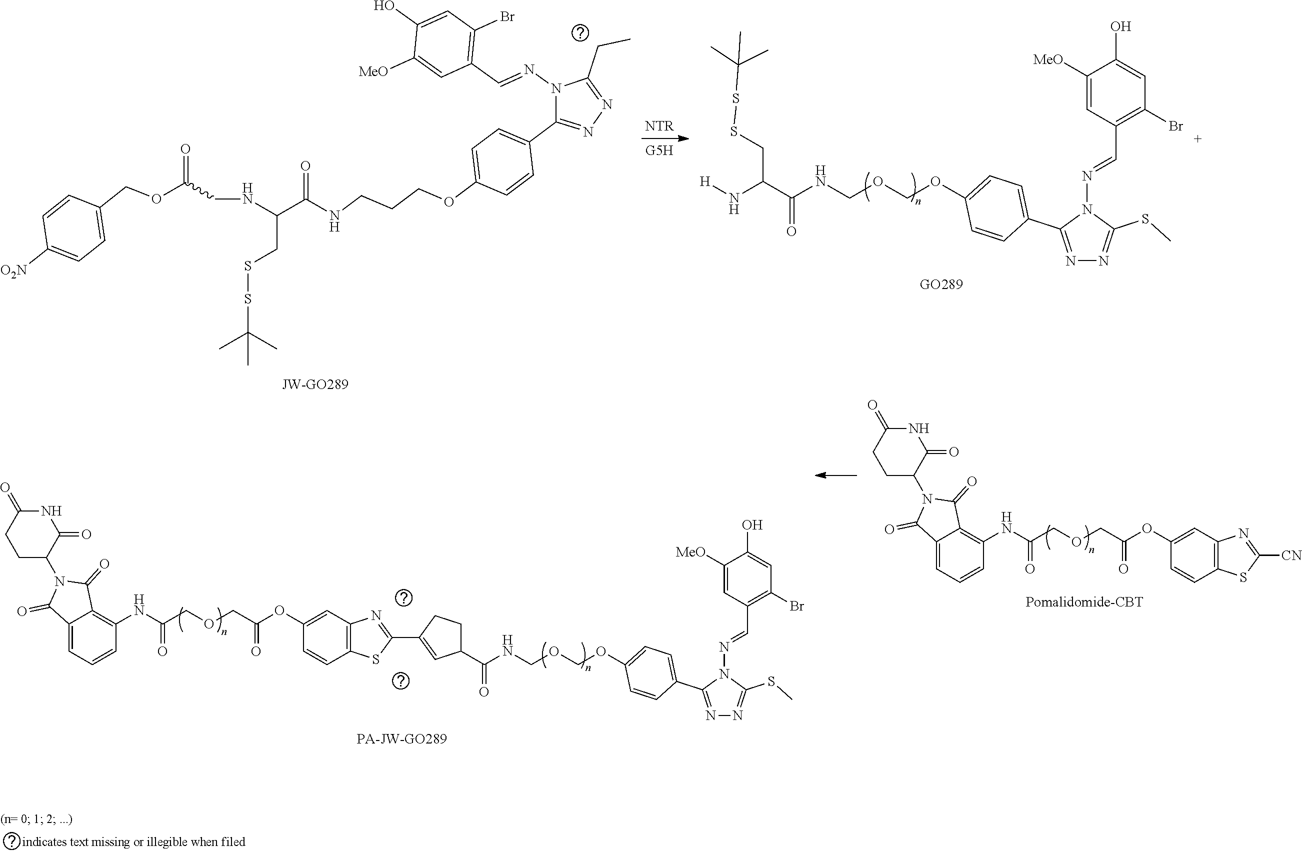

PA-JW-GO289 was prepared from GO289 molecule by following the protocol for J252 in Example 9.

### Example 9. Synthesis of Enzyme Responsive Peptide PROTAC (ENCTAC) Moieties with Different Linkage Distance for Degradation of BRD4

Besides enzyme (e.g. furin, and etc.) controlled cross-linking of pomalidomide and GO289 for specific degradation of tumour circadian CK2α kinase protein, such enzymatic reactions can be used for any type of PROTACs system. For instance, pomalidomide (E3 ligase moiety) can also covalently cross-link with JQ1, a thienotriazolodiazepine and potent inhibitor for the bromodomain and extra-terminal (BET) family of bromodomain proteins in mammals, including BRD2, BRD3, BRD4, and etc. Similar to the structures and reactions in FIGS. 5 and 6, the enzyme triggered targeted cross-linking can be also used for the degradation of BRD4.

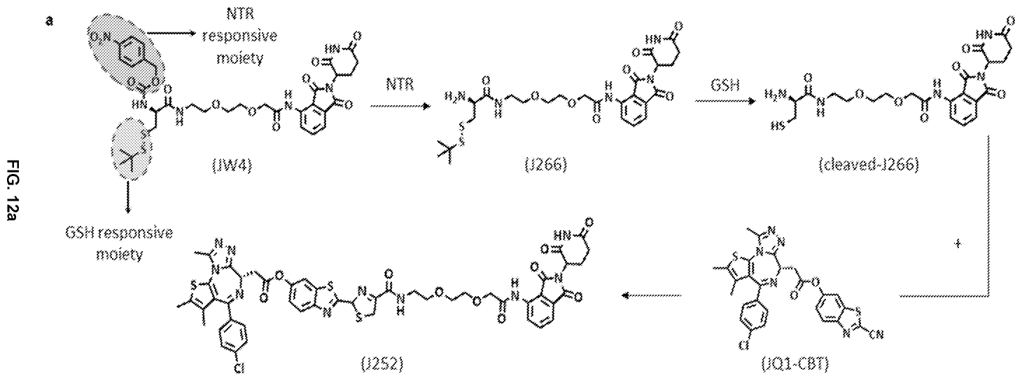

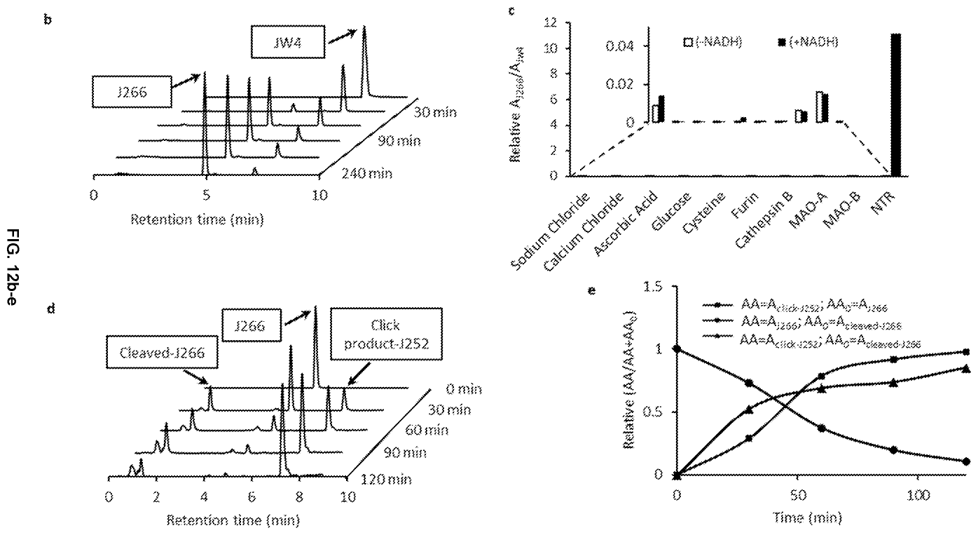

We developed an extraordinary system that offers ENCTACs for specific protein degradations in the hypoxic microenvironment (FIG. 10). Typically, such degradation paradigm relies on NTR for selective formation of heterobifunctional degraders at the hypoxic site only, thus promoting targeted epigenetic BRD4 degradation in vitro and in vivo. The ENCTAC is initialized by NTR reduction of nitro-containing substrate to expose the cysteine fragment that has been pre-modified with an E3 ligase CRBN recruiting moiety, pomalidomide. Subsequently, the orthogonal cross-linking occurs between the uncaged cysteine residue with the CBT group pre-conjugated with the BRD4 ligand, JQ1. This first-in-class strategy realizes enzyme selective protein degradation without tedious chemical synthesis to link two protein recruiters beforehand, as always observed in conventional PROTAC design. Such small molecule ENCTAC conjugation can not only achieve precise manipulation of hypoxia pathways in complex living settings, but more importantly, it can also largely facilitate new drug development and high throughput screening of relevant therapeutic reagents with minimal concerns of toxicity, off-target effects, and resistance. Such game-changing design offers compelling superiorities to supply PROTAC technology with more flexible practicality and druggable potency for precision medicine in the future.

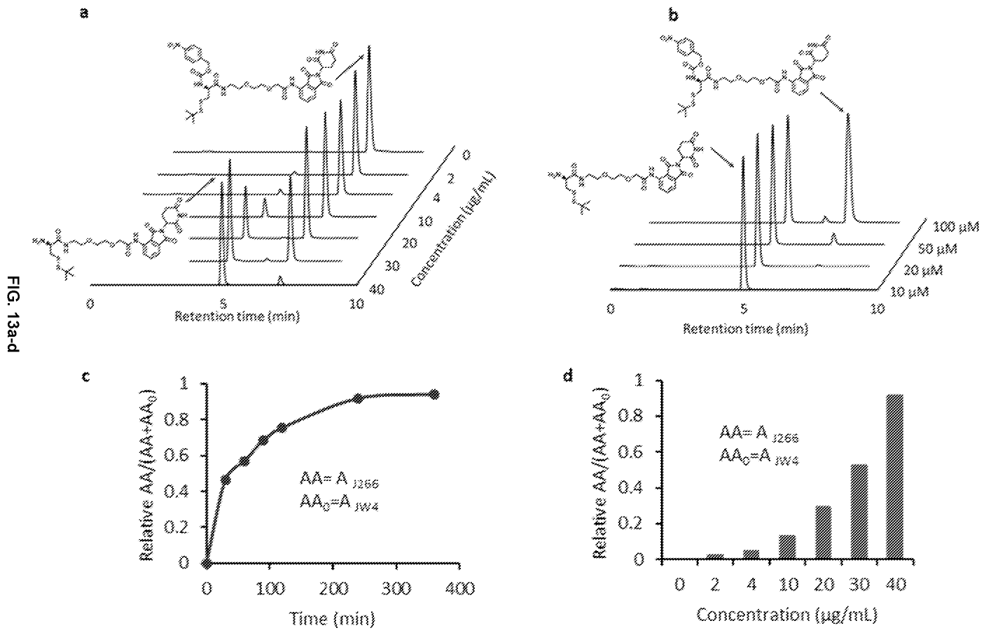

To optimize the distance, different PEG moieties were used for modification with the E3 ligase ligand (pomalidomide), and degradation recognition moiety (JQ1), as shown in FIG. 11.

### Synthesis of J242 and J252

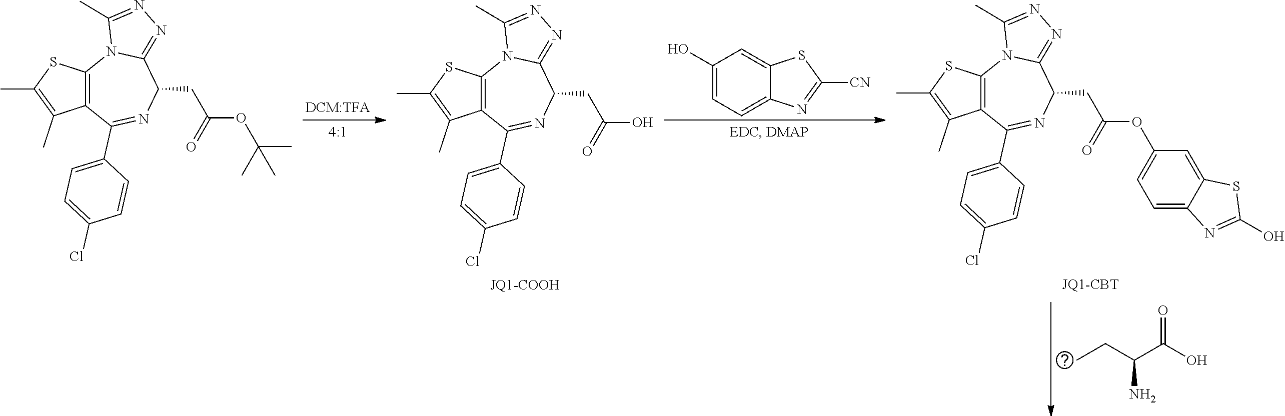

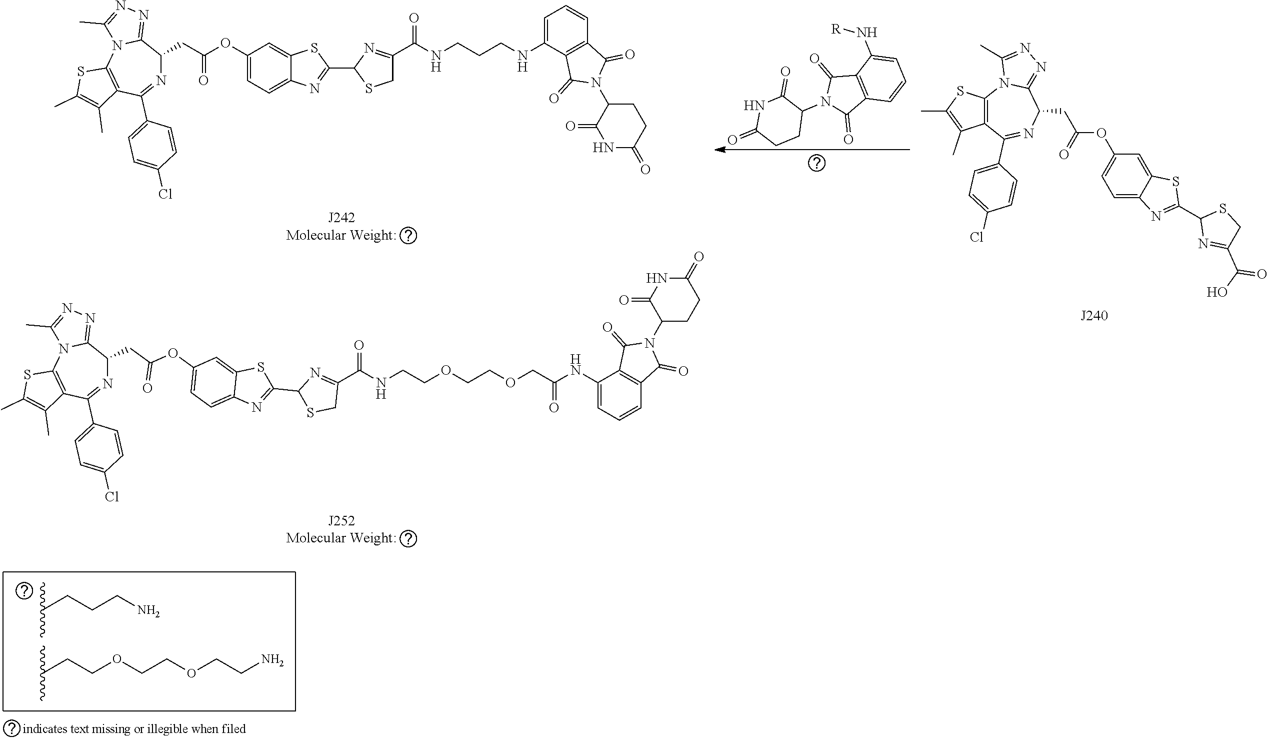

### JQ1-COOH

To a 50-mL round bottom flask, (+)-JQ1 (40 mg, 0.0998 mmol) was added, which was subsequently dissolved in a mixture of 1:1—DCM:trifluoroacetic acid (TFA, 4 mL). The reaction was stirred at r.t for 20 min, which was then extracted with deionised (DI) H₂O (1×) and dichloromethane (DCM, 2×), dried in magnesium sulfate (MgSO₄), filtered and evaporated in vacuo to yield the deprotected-(+)-JQ1. The organic extract was used for the subsequent synthetic step without further purification.

ESI-MS: [M]⁺ 401.20.

### JQ1-CBT

Deprotected-(+)-JQ-1 (18 mg, 0.045 mmol) was dissolved in DCM and stirred together with 4-dimethylaminopyridine (2.7 mg, 0.0225 mmol) and 6-hydroxybenzothiazole-2-carbonitrile (31.7 mg, 0.18 mmol) for 30 min at r.t. Thereafter, 1-ethyl-3-(3-dimethylaminopropyl)carbodiimide (9.4 mg, 0.049 mmol) pre-dissolved in DMF (0.5 mL) was added dropwise into the reaction mixture and stirred for 4 h at r.t. The resultant crude product was purified by RP-HPLC, and lyophilized to yield JQ1-CBT as a pale-yellowish solid (14.8 mg, 58.8%).

¹H NMR (400 MHZ, MeOD) δ 8.25 (d, J=9.0 Hz, 1H), 8.05 (d, J=2.3 Hz, 1H), 7.54 (dd, J=9.0, 2.3 Hz, 1H), 7.49 (d, J=8.6 Hz, 2H), 7.44 (d, J=8.7 Hz, 2H), 4.74 (t, J=7.2 Hz, 1H), 3.92-3.85 (d, 2H), 2.72 (s, 3H), 2.45 (s, 3H), 1.70 (s, 3H). ¹³C NMR (100 MHZ, MeOD) δ 171.99, 166.60, 156.60, 152.39, 152.26, 151.45, 139.41, 138.16, 138.01, 137.73, 133.61, 133.45, 132.01, 131.91, 131.35, 129.90, 126.58, 124.25, 116.45, 113.83, 53.62, 37.51, 14.39, 12.51, 10.05. ESI-MS: [M+H]⁺ 559.28.

### JQ1-CBT-Cys (J240)

To JQ1-CBT (4 mg, 0.0072 mmol) dissolved in DMF (0.5 mL), L-cysteine (8 mg, 0.066 mmol) in a solution of H₂O (0.5 mL) was added, with the solvent proportion of 1:1. The reaction was stirred at r.t. for 2 h to complete the reaction. The resultant crude product was extracted with DI water (1×) and DCM (2×), dried in MgSO₄, filtered and evaporated in vacuo to yield a light-yellow solid J240 (55% yield). The organic extract was used in the subsequent synthetic step without further purification.

¹H NMR (400 MHZ, MeOD) δ 8.13 (d, J=8.0 Hz, 1H), 7.93 (d, J=2.2 Hz, 1H), 7.50 (d, J=8.0 Hz, 2H), 7.46-7.39 (m, 3H), 5.43 (t, J=9.2 Hz, 1H), 4.77 (t, J=7.2 Hz, 1H), 3.87 (d, J=7.2 Hz, 2H), 3.80 (dd, J=9.2, 2.3 Hz, 2H), 2.75 (s, 3H), 2.46 (s, 3H), 1.71 (s, 3H). ¹³C NMR (101 MHZ, MeOD) δ 173.15, 171.09, 167.52, 166.67, 162.75, 156.60, 154.71, 152.31, 151.15, 138.24, 138.00, 137.90, 133.69, 133.42, 132.10, 132.06, 131.39, 129.92, 125.83, 122.93, 116.37, 79.59, 54.77, 37.45, 35.97, 14.39, 12.93, 11.57. ESI-MS: [M]+663.05.

### J242

Crude J240 (4 mg, 0.00603 mmol) was added to 2-(1H-benzotriazole-1-yl)-1,1,3,3-tetramethyluronium hexafluorophosphate (HBTU, 18.36 mg, 0.0484 mmol), and the reaction mixture was left to stir for 30 min at r.t. After that, pomalidomide-C₃—NH₂ (2.2 mg, 0.00663 mmol) was dissolved in DMF (0.5 mL) and added dropwise into the reaction mixture, with subsequent addition of N, N-diisopropylethylamine (DIPEA, 8.4 μL, 0.0484 mmol) to react for 6 h at ambient condition. The resulting product was purified by RP-HPLC, and lyophilized to yield J242 as a yellowish solid (2.2 mg, 37.4%).

¹H NMR (600 MHZ, DMSO-d₆) δ 11.08 (s, 1H), 8.27 (t, J=5.7 Hz, 1H), 8.22 (d, J=8.9 Hz, 1H), 8.06 (t, J=2.1 Hz, 1H), 7.52 (d, J=8.9 Hz, 2H), 7.48 (d, J=8.5 Hz, 2H), 7.40 (dd, J=8.9, 2.3 Hz, 1H), 7.08 (d, J=8.6 Hz, 1H), 7.00 (d, J=6.7 Hz, 1H), 5.35 (t, J=9.3 Hz, 1H), 5.04 (dd, J=12.8, 5.4 Hz, 2H), 4.67-4.62 (m, 1H), 3.84-3.69 (m, 1H), 3.27-3.23 (m, 2H), 2.90-2.84 (m, 2H), 2.63 (s, 3H), 2.61-2.57 (m, 1H), 2.41 (s, 3H), 2.04-2.00 (m, 1H), 1.77-1.73 (m, 2H), 1.63 (s, 3H). ¹³C NMR (151 MHZ, DMSO) δ 172.86, 170.14, 169.63, 169.11, 168.84, 167.34, 164.27, 163.67, 161.31, 154.60, 150.52, 150.18, 149.17, 146.28, 136.62, 136.27, 135.45, 132.40, 130.91, 130.29, 129.95, 129.51, 128.62, 124.83, 121.97, 118.12, 117.13, 115.78, 110.42, 109.21, 79.35, 53.46, 48.55, 34.74, 31.00, 28.60, 22.18, 14.09, 12.70, 11.34. ESI-MS: [M]⁺ 975.29.

### J252

J252 was prepared from 4-((2-(2-(2-aminoethoxy)ethoxy)ethyl)amino)-2-(2,6-dioxopiperidin-3-yl)isoindoline-1,3-dione by following the protocol for J242.

¹H NMR (600 MHZ, DMSO-d₆) δ 11.14 (s, 1H), 10.33 (s, 1H), 8.68 (d, J=8.4 Hz, 1H), 8.19 (d, J=8.9 Hz, 1H), 8.00 (t, J=2.4 Hz, 1H), 7.84-7.78 (m, 1H), 7.58 (dd, J=7.3, 3.6 Hz, 1H), 7.59-7.57 (m, 4H), 7.39-7.35 (m, 1H), 5.33 (td, J=9.1, 2.4 Hz, 1H), 5.16 (dd, J=12.9, 5.4 Hz, 1H), 4.66 (t, J=7.2 Hz, 1H), 4.19 (s, 2H), 3.86-3.69 (m, 8H), 3.54-3.52 (m, 2H), 3.36-3.30 (m, 2H), 2.99-2.96 (m, 1H), 2.93-2.83 (m, 2H), 2.64 (s, 3H), 2.41 (s, 3H), 2.13-2.02 (m, 1H), 1.63 (s, 3H). ¹³C NMR (151 MHZ, DMSO) δ 172.77, 169.78, 169.53, 169.35, 169.16, 168.26, 166.70, 164.33, 163.76, 161.20, 154.52, 150.48, 150.32, 149.12, 136.52, 136.10, 135.95, 135.49, 132.24, 131.28, 131.09, 130.32, 130.00, 129.62, 128.61, 124.79, 124.36, 121.91, 118.31, 116.02, 115.69, 79.14, 70.74, 70.20, 69.51, 68.84, 53.39, 48.99, 48.62, 36.57, 30.96, 28.26, 21.99, 14.09, 12.72, 11.33. ESI-MS: [M+H]⁺ 1063.32.

### Synthesis of T208

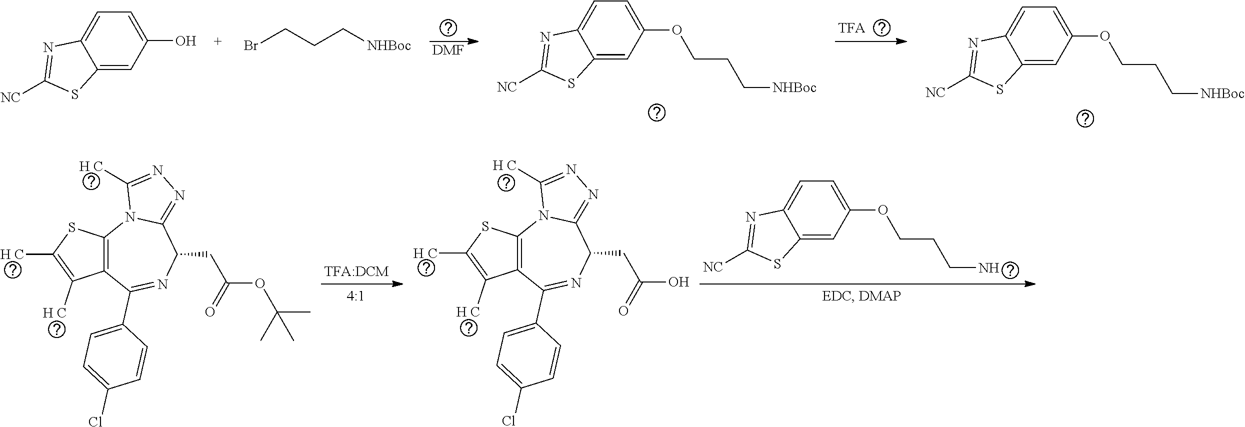

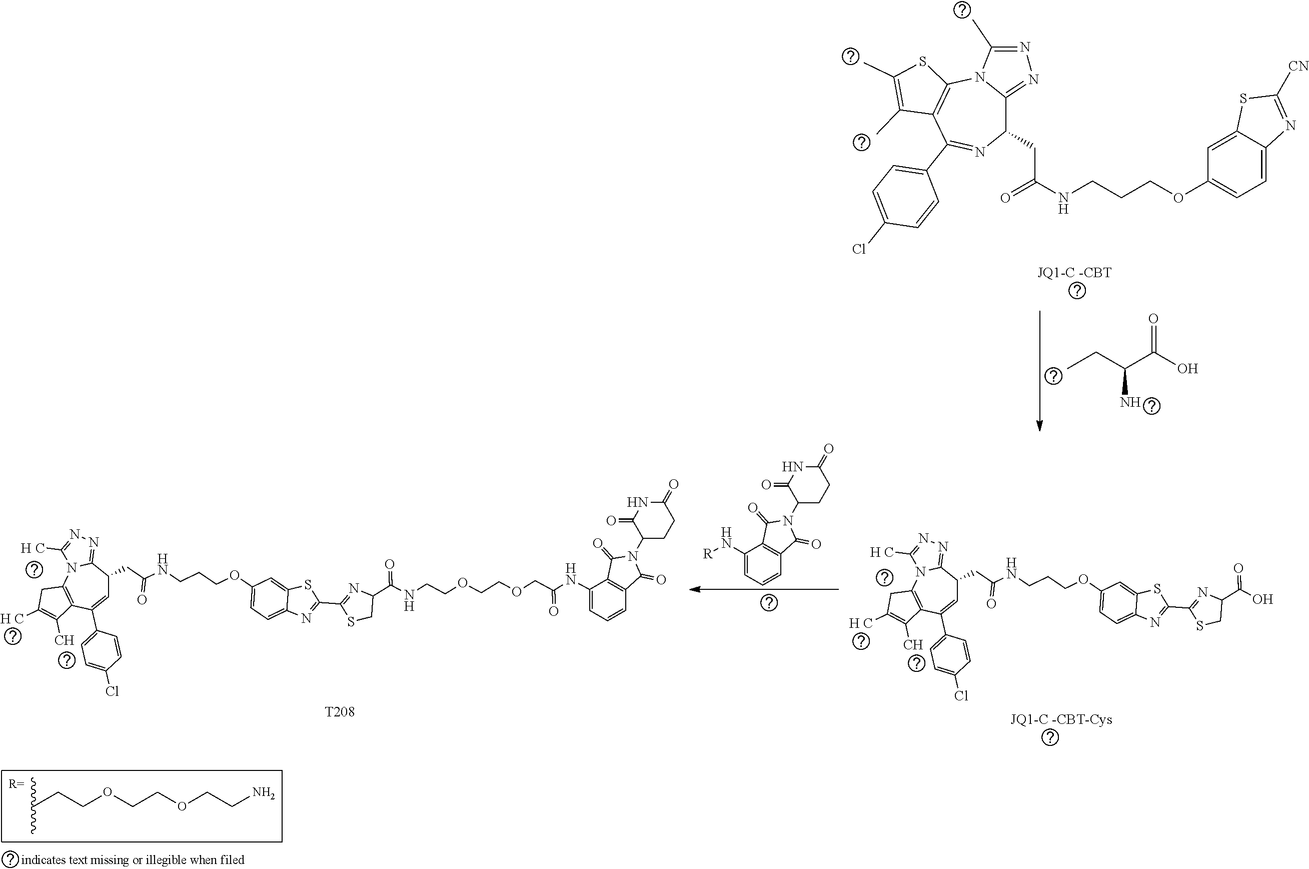

### BocNH—C₃—CBT

To a stirred solution of 6-hydroxybenzo[d]thiazole-2-carbonitrile (50 mg, 0.284 mmol) in DMF (1 mL), N-ethyldiisopropylamine in 1-methyl-2-pyrrolidinone (0.341 mmol, 171 μL, 2 M) was added. The reaction mixture was stirred for 10 min. Then, the solution of tert-butyl (3-bromopropyl)carbamate (134 mg, 0.570 mmol) in DMF (1 mL) was added into the reaction mixture. The reaction mixture was stirred at r.t for 16 h. The crude product was purified by RP-HPLC. The product-containing fractions were dried by lyophilisation to afford BocNH—C₃—CBT as a yellow solid (23.7 mg, 25%).

¹H NMR (400 MHZ, MeOD) δ 8.04 (d, J=9.1 Hz, 1H), 7.62 (d, J=2.5 Hz, 1H), 7.28 (dd, J=9.1, 2.5 Hz, 1H), 4.13 (t, J=6.1 Hz, 2H), 3.26 (t, J=6.8 Hz, 2H), 2.00 (p, J=6.4 Hz, 2H), 1.42 (s, 9H). ¹³C NMR (100 MHZ, MeOD) δ 161.34, 158.56, 148.05, 138.98, 134.92, 126.40, 120.16, 114.19, 105.22, 80.00, 67.51, 54.79, 38.27, 30.55, 28.74. ESI-MS: [M+H]⁺ 234.04.

NH₂—C₃—CBT

To a stirred solution of BocNH—C₃—CBT (23.7 mg, 71.0 μmol) in DCM (1 mL), TFA (0.5 mL, 6.53 mmol) was added. The reaction mixture was stirred for 1 h at r.t. The crude product was purified by RP-HPLC. The product-containing fractions were dried by lyophilisation to afford NH₂—C₃—CBT as a light yellow liquid (14.3 mg, 86%).

¹H NMR (400 MHZ, MeOD) δ 8.09 (d, J=9.1 Hz, 1H), 7.69 (d, J=2.5 Hz, 1H), 7.34 (dd, J=9.1, 2.5 Hz, 1H), 4.25 (t, J=5.8 Hz, 2H), 3.19 (t, J=7.3 Hz, 2H), 2.22 (ddd, J=13.0, 7.3, 5.7 Hz, 2H). ¹³C NMR (100 MHZ, MeOD) δ 160.79, 148.35, 138.98, 135.40, 126.57, 120.01, 114.12, 105.46, 67.08, 38.45, 28.22. ESI-MS: [M+H]+ 234.04.

### JQ1-C₃—CBT

To a stirred solution of NH₂—C₃—CBT (14.3 mg, 61.3 μmol) and JQ1-COOH (29.5 mg, 73.6 μmol) in DMF (800 μL), 4-dimethylaminopyridine (0.750 mg, 6.13 μmol) was added. Then, 1-ethyl-3-(3-dimethylaminopropyl)carbodiimide (14.1 mg, 73.6 μmol) was added into the reaction mixture. The reaction mixture was stirred for 16 h at r.t. The crude product was purified by RP-HPLC. The product-containing fractions were dried by lyophilisation to afford JQ1-C₃—CBT as light-yellow solid (11.7 mg, 31%).

¹H NMR (400 MHZ, MeOD) δ 8.05 (d, J=9.1 Hz, 1H), 7.64 (d, J=2.5 Hz, 1H), 7.44-7.36 (m, 2H), 7.34-7.21 (m, 3H), 4.65 (dd, J=8.7, 5.6 Hz, 1H), 4.27-4.10 (m, 2H), 3.58-3.36 (m, 4H), 2.69 (s, 3H), 2.48-2.37 (m, 3H), 2.11 (t, J=6.4 Hz, 2H), 1.66 (s, 3H). ¹³C NMR (100 MHz, MeOD) δ 173.56, 166.46, 162.06, 156.94, 152.57, 149.07, 139.02, 138.10, 137.78, 134.99, 133.52, 133.45, 132.11, 131.98, 131.29, 129.75, 126.44, 120.25, 113.57, 105.39, 66.58, 56.06, 40.87, 37.25, 30.59, 13.98, 12.48, 11.57. ESI-MS: [M+H]⁺ 616.28.

### JQ1-C₃—CBT-Cys

To a stirred solution of JQ1-C₃—CBT (11.7 mg, 19.0 μmol) in DMF (800 μL), L-cysteine (2.30 mg, 19.0 μmol) was added. The reaction mixture was heated to 40° C. and stirred for 2 h. The reaction mixture was then cooled down to r.t. and purified by RP-HPLC. The product-containing fractions were dried by lyophilisation to afford JQ1-C₃—CBT-Cys as a white solid (8.61 mg, 63%).

¹H NMR (400 MHZ, MeOD) δ 7.92 (d, J=12.0 Hz, 1H), 7.53 (d, J=4.0 Hz, 1H), 7.39 (d, J=8.0 Hz, 2H), 7.28 (d, J=8.0 Hz, 2H), 7.18 (dd, J=12.0 Hz, 4.0 Hz, 1H), 5.40 (t, J=8.0 Hz, 1H), 4.64 (t, J=8.0 Hz, 1H), 4.21-4.11 (m, 2H), 3.81-3.73 (m, 2H), 3.52-3.40 (m, 4H), 2.68 (s, 3H), 2.43 (s, 3H), 2.13-2.07 (m, 2H), 1.66 (s, 3H). ¹³C NMR (101 MHZ, MeOD) δ 173.27, 172.86, 167.63, 166.46, 160.24, 159.45, 156.94, 152.30, 148.85, 139.06, 138.13, 137.75, 133.50, 133.44, 132.12, 131.97, 131.27, 129.76, 125.78, 118.70, 105.84, 79.48, 67.33, 55.13, 38.72, 37.27, 35.88, 30.20, 14.38, 12.92, 11.57. ESI-MS: [M+H]⁺ 720.29.

### T208

To a stirred solution of JQ1-C₃—CBT-Cys (4.60 mg, 6.39 μmol) and HBTU (3.63 mg, 9.59 μmol) in DMF (500 μL), Poma-(PEG)₂-NH₂·HCl (2.90 mg, 6.39 μmol) was added. N-Ethyldiisopropylamine in 1-methyl-2-pyrrolidinone (9.59 μmol, 4.75 μL, 2 M) was then added into the reaction mixture. The reaction mixture was stirred at r.t for 18 h. The crude product was purified by RP-HPLC. The product-containing fractions were dried by lyophilisation to afford T208 as a white solid (3.07 mg, 43%).

¹H NMR (600 MHZ, DMSO-d₆) δ 11.14 (s, 1H), 10.33 (s, 1H), 8.68 (dd, J=8.4, 2.6 Hz, 1H), 8.34 (t, J=5.6 Hz, 1H), 8.01-7.96 (m, 1H), 7.85-7.80 (m, 1H), 7.59 (d, J=7.2 Hz, 1H), 7.45-7.36 (m, 4H), 7.21-7.14 (m, 1H), 5.29 (td, J=9.3, 2.4 Hz, 1H), 5.18-5.11 (m, 1H), 4.53 (t, J=7.1 Hz, 1H), 4.20-4.08 (m, 4H), 3.81-3.59 (m, 10H), 3.33-3.30 (m, 2H), 3.25 (t, J=7.1 Hz, 2H), 2.94-2.84 (m, 2H), 2.61 (s, 3H), 2.39 (s, 3H), 2.12-2.02 (m, 2H), 1.97-1.94 (m, 2H), 1.58 (s, 3H). ¹³C NMR (151 MHZ, DMSO) δ 172.90, 172.31, 170.03, 169.91, 169.61, 169.52, 168.49, 166.93, 164.76, 163.44, 158.44, 155.39, 150.17, 147.26, 137.41, 136.93, 136.68, 136.05, 135.52, 132.43, 131.47, 131.03, 130.28, 130.13, 129.88, 128.63, 124.90, 124.48, 118.49, 117.55, 105.37, 79.21, 70.99, 70.41, 69.74, 69.04, 66.18, 54.09, 49.19, 37.87, 35.52, 34.84, 31.11, 29.07, 22.20, 21.06, 14.12, 12.76, 11.40. ESI-MS: [M+H]+ 1120.32.

### Synthesis of A and B

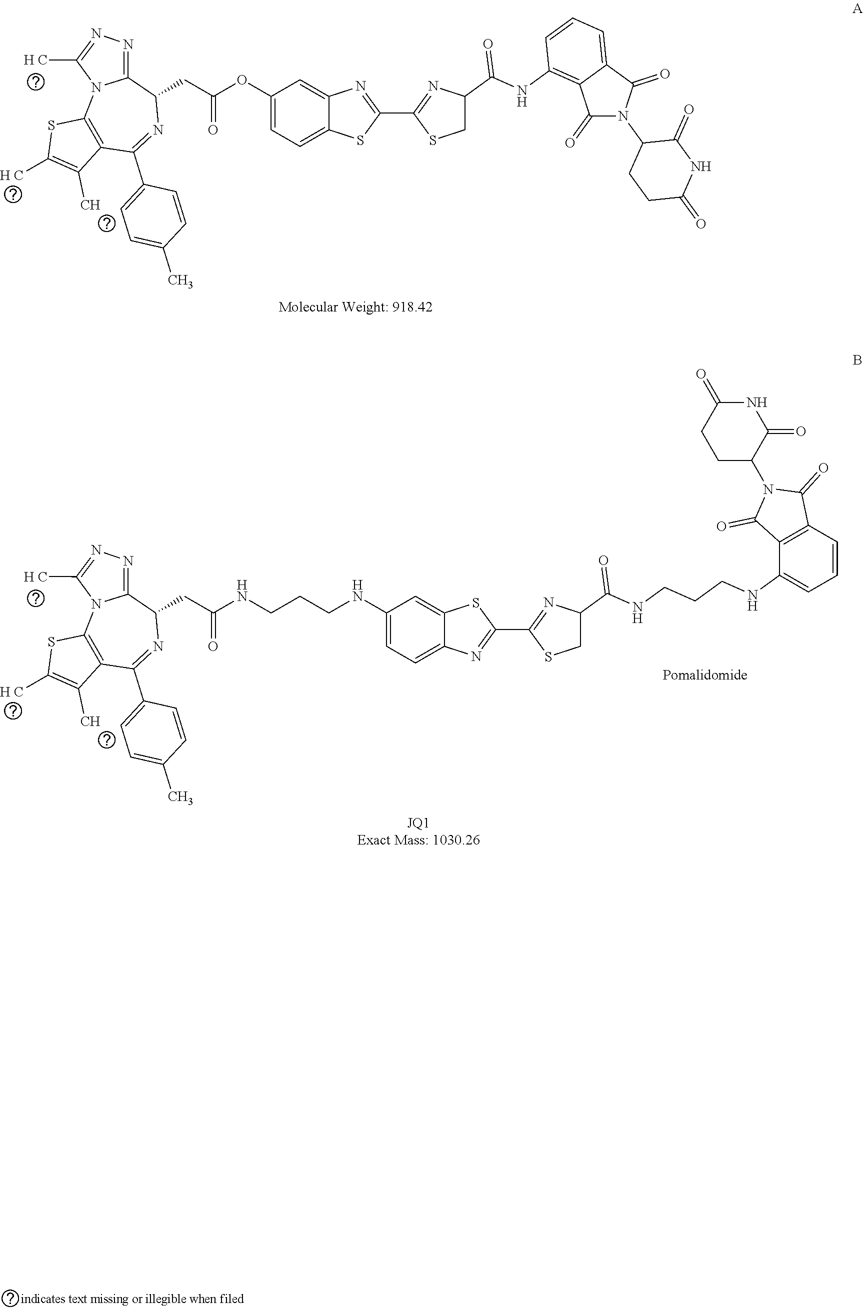

Compounds A and B were prepared by following the protocol for T208.

Synthesis of pomalidomide-C3-NTR (J268)

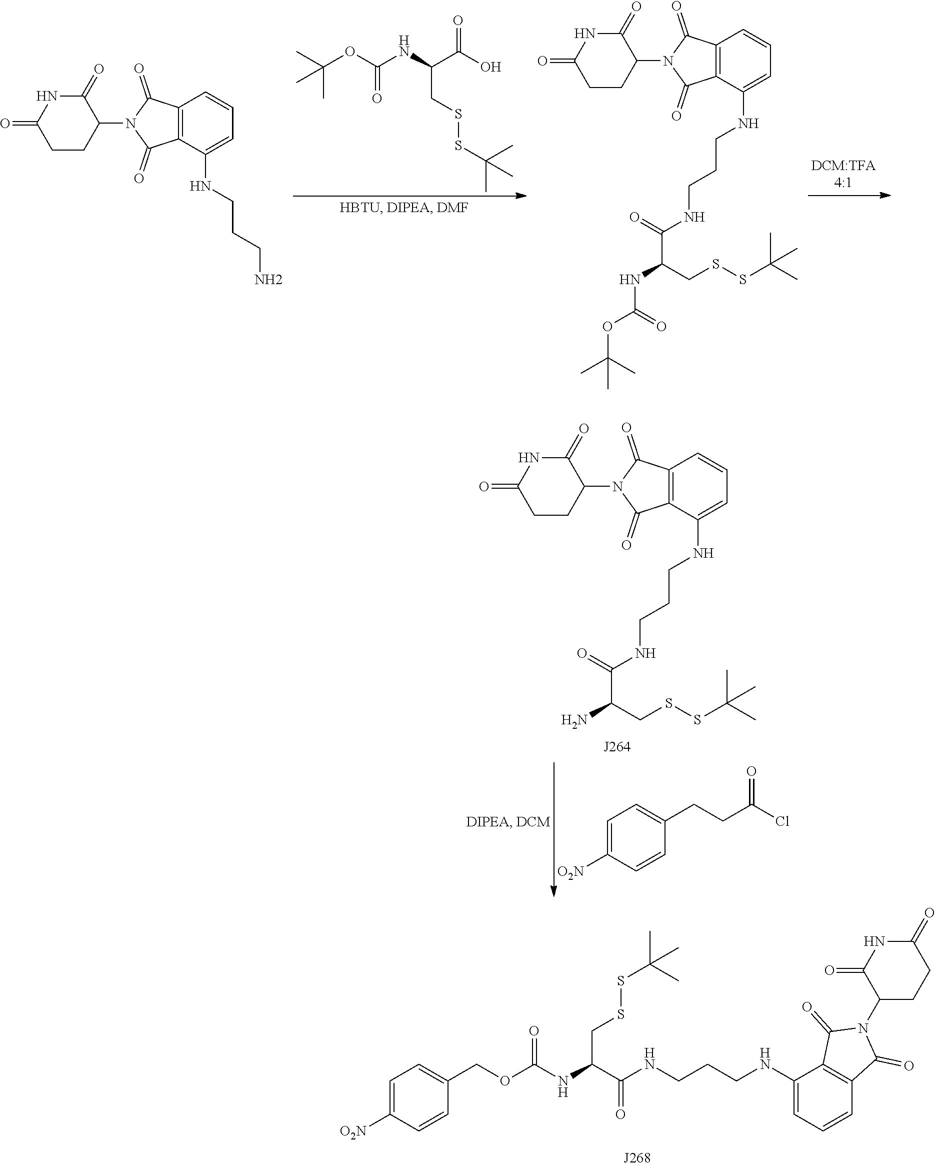

### J260

To a 50-mL round bottom flask, Boc-Cys(StBu)—OH (6.75 mg, 0.0218 mmol) and HBTU (27.6 mg, 0.073 mmol) in DMF (0.5 mL) were added. The reaction mixture was left to stir for 30 min at r.t. Thereafter, pomalidomide-C₃—NH₂ (6 mg, 0.0182 mmol) was dissolved in DMF (0.5 mL) and added dropwise into the reaction mixture, followed by the addition of N,N-diisopropylethylamine (12.9 μL, 0.073 mmol). The reaction mixture was stirred at 50° C. for 4 h. The resulting crude product was purified by RP-HPLC, and lyophilized to yield J260 as a yellowish solid (6.7 mg, 59.3%).

¹H NMR (400 MHZ, MeOD) δ 7.52 (dd, J=8.5, 7.0 Hz, 1H), 7.03 (t, J=7.7 Hz, 2H), 5.05 (dd, J=12.5, 5.4 Hz, 1H), 4.31 (t, J=7.0 Hz, 1H), 3.37 (t, J=6.8 Hz, 2H), 3.35-3.26 (m, 4H), 3.10 (dd, J=13.4, 5.8 Hz, 1H), 3.00-2.80 (m, 2H), 2.79-2.65 (m, 2H), 2.11 (ddq, J=10.3, 5.5, 2.2 Hz, 1H), 1.84 (p, J=6.8 Hz, 2H), 1.44 (s, 9H), 1.33 (s, 9H). ¹³C NMR (100 MHZ, MeOD) δ 174.69, 173.33, 171.63, 170.68, 169.32, 157.54, 148.04, 137.23, 133.92, 118.07, 111.84, 111.19, 80.90, 55.80, 50.18, 43.48, 40.88, 37.95, 32.22, 30.21, 30.03, 28.70, 23.79. ESIMS: [M]⁺ 622.15; [M+2H]²⁺ 1242.68.

### J264

J260 (6.0 mg) was dissolved in a solution mixture of DCM:TFA—4:1 and stirred for 20 min at r.t. The crude product was then extracted with DI H₂O (1×) and DCM (2×), dried in MgSO₄, filtered and evaporated in vacuo. Further purification was performed by RP-HPLC, and lyophilisation was performed to yield J264 as a yellowish solid (5.4 mg, 96.3%).

¹H NMR (400 MHZ, MeOD) δ 7.56 (dd, J=8.6, 7.1 Hz, 1H), 7.07 (dd, J=7.8, 4.5 Hz, 2H), 5.06 (dd, J=12.5, 5.5 Hz, 1H), 4.06 (dd, J=7.2, 6.1 Hz, 1H), 3.42 (dd, J=6.9, 4.6 Hz, 2H), 3.40-3.33 (m, 2H), 3.27-3.05 (m, 2H), 2.93-2.81 (m, 1H), 2.80-2.65 (m, 2H), 2.18-2.06 (m, 1H), 1.90 (p, J=6.7 Hz, 2H), 1.35 (s, 9H). ¹³C NMR (100 MHZ, MeOD) δ 174.67, 171.69, 170.79, 169.27, 168.64, 148.02, 137.31, 133.97, 118.01, 112.00, 111.30, 54.03, 50.19, 42.33, 41.00, 38.43, 32.19, 30.07, 29.82, 23.80. ESI-MS: [M]⁺ 522.32.

### J268

J264 (3.2 mg, 0.00614 mmol) was added to a solution mixture of p-nitrobenzyl chloroformate (2.65 mg, 0.0123 mmol) and N,N-diisopropylethylamine (4.4 μL, 0.0246 mmol) dissolved in DCM (0.5 mL). The reaction was completed upon 2 h of stirring at r.t. The resultant crude product was then purified by RP-HPLC, and was lyophilized to yield J268 as a yellowish solid (2.8 mg, 65.1%).

¹H NMR (400 MHZ, MeOD) δ 8.19-8.14 (m, 2H), 7.57 (d, J=8.5 Hz, 2H), 7.50 (dd, J=8.6, 7.1 Hz, 1H), 7.00 (dd, J=7.9, 6.0 Hz, 2H), 5.23 (d, J=2.5 Hz, 2H), 5.05 (dd, J=12.5, 5.4 Hz, 1H), 4.43-4.32 (m, 1H), 3.05 (ddd, J=70.6, 13.5, 7.1 Hz, 2H), 2.89-2.79 (m, 1H), 2.79-2.65 (m, 2H), 2.12 (td, J=7.0, 6.4, 3.0 Hz, 1H), 1.83 (h, J=6.6 Hz, 2H), 1.32 (s, 9H). ¹³C NMR (100 MHZ, MeOD) δ 174.73, 173.01, 171.66, 170.67, 169.34, 157.82, 148.81, 147.98, 145.93, 137.22, 133.91, 129.01, 128.25, 124.56, 124.45, 118.03, 111.82, 111.13, 66.36, 56.27, 50.18, 43.12, 40.85, 37.98, 32.21, 30.20, 29.99, 23.79. ESI-MS: [M]⁺701.14.

### Synthesis of Pomalidomide-PEG-NTR (JW4)

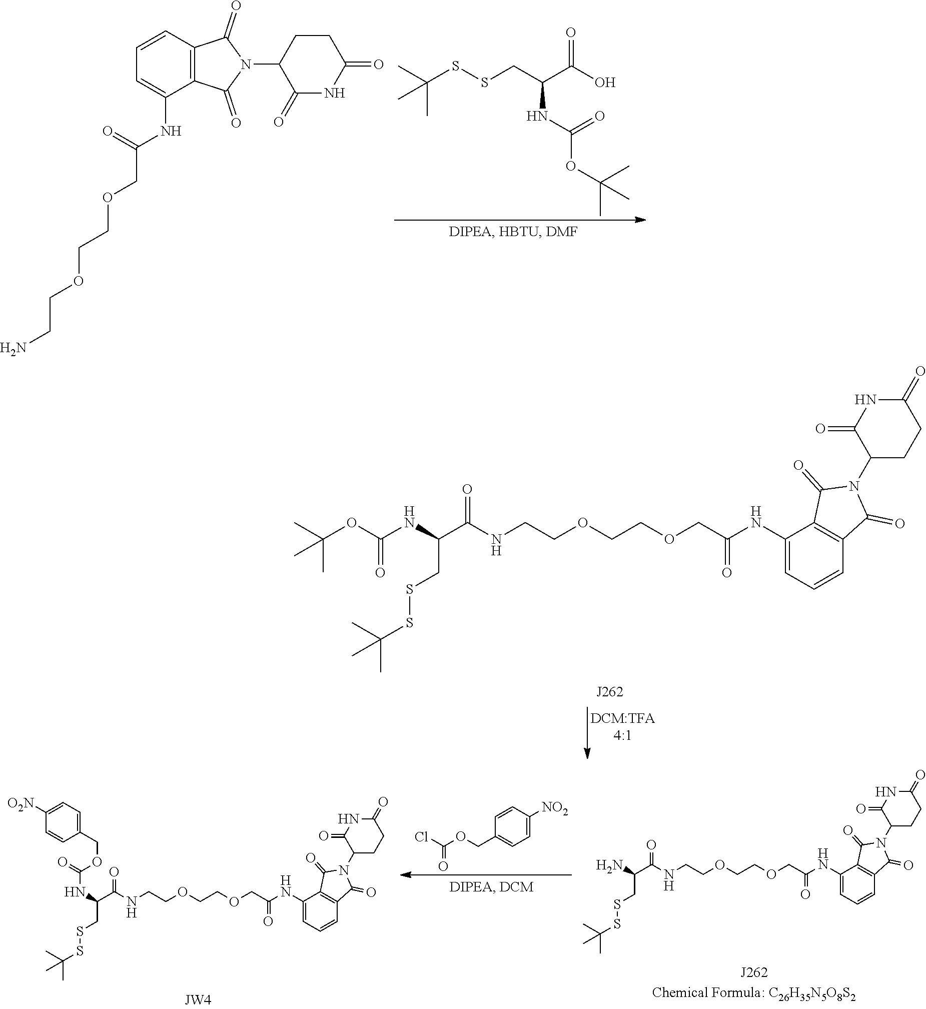

### J262

J262 (white solid, 9.3 mg, 54.8%) was prepared by following the protocol for J260 except Boc-Cys(StBu)—OH (8.9 mg, 0.0287 mmol), HBTU (36.3 mg, 0.096 mmol) in DMF, N,N-diisopropylethylamine (17 μL, 0.096 mmol), and pomalidomide-PEG₂-NH₂ (10 mg, 0.0239 mmol) instead of pomalidomide-C₃—NH₂, were used.

¹H NMR (400 MHZ, MeOD) δ 8.81 (d, J=8.5 Hz, 1H), 7.81 (t, J=7.9 Hz, 1H), 7.61 (d, J=7.3 Hz, 1H), 5.16 (dd, J=12.6, 5.4 Hz, 1H), 4.30 (s, 1H), 4.22 (s, 2H), 3.88-3.71 (m, 4H), 3.61 (t, J=5.6 Hz, 2H), 3.42 (tq, J=19.2, 6.9, 6.2 Hz, 2H), 3.09 (dd, J=13.5, 5.4 Hz, 1H), 2.95-2.84 (m, 2H), 2.83-2.64 (m, 2H), 2.28-2.11 (m, 1H), 1.43 (s, 9H), 1.30 (s, 9H). ¹³C NMR (100 MHZ, MeOD) δ 174.71, 173.14, 171.60, 171.35, 169.90, 168.34, 157.56, 137.50, 137.39, 132.99, 125.96, 119.70, 117.89, 80.99, 72.63, 71.74, 71.25, 70.49, 55.58, 54.79, 50.58, 43.56, 40.44, 32.13, 30.18, 28.67, 23.65. ESI-MS: [M+H]⁺ 710.09; [M+Na]⁺732.30.

### J266

J266 (white solid, 7.3 mg, 91.4%) was prepared by following the protocol for J264 except J262 instead of J260 was used.

¹H NMR (400 MHZ, MeOD) δ 8.82 (d, J=8.4 Hz, 1H), 7.82 (t, J=7.9 Hz, 1H), 7.62 (d, J=7.3 Hz, 1H), 5.16 (dd, J=12.3, 5.0 Hz, 1H), 4.22 (s, 2H), 4.10-4.00 (m, 1H), 3.88-3.74 (m, 4H), 3.64 (d, J=6.0 Hz, 2H), 3.57-3.34 (m, 2H), 3.25-3.01 (m, 2H), 2.90 (ddd, J=18.5, 13.8, 5.2 Hz, 1H), 2.83-2.67 (m, 2H), 2.18 (d, J=12.7 Hz, 1H), 1.32 (s, 9H). ¹³C NMR (100 MHZ, MeOD) δ 174.72, 171.56, 169.98, 168.55, 168.22, 137.55, 137.42, 133.04, 125.93, 119.71, 117.88, 72.62, 71.77, 71.38, 70.37, 53.88, 50.58, 42.22, 40.81, 32.12, 30.14, 30.04, 23.64. ESIMS: [M+H]⁺ 610.30.

### JW4

JW4 (white solid, 5.4 mg, 41.9%) was prepared by following the protocol for J268 except p-nitrobenzyl chloroformate (7.1 mg, 0.0328 mmol), N,N-diisopropylethylamine (22.8 μL, 0.1312 mmol) dissolved in DCM (0.5 mL), and J266 (10 mg, 0.0164 mmol) instead of J264, were used.

¹H NMR (400 MHZ, MeOD) δ 8.78-8.69 (m, 1H), 8.16-8.10 (m, 2H), 7.74 (dd, J=8.5, 7.3 Hz, 1H), 7.54 (d, J=4.8 Hz, 1H), 7.54-7.50 (m, 2H), 5.15-5.13 (m, 1H), 4.55 (s, 2H), 4.35 (dt, J=9.1, 4.9 Hz, 1H), 4.16 (s, 2H), 3.79-3.67 (m, 4H), 3.55 (d, J=5.5 Hz, 2H), 3.38-3.33 (m, 2H), 3.17-3.06 (m, 2H), 2.93-2.84 (m, 2H), 2.82 (d, J=5.3 Hz, 1H), 2.76-2.66 (m, 2H), 2.63 (s, 1H), 2.18-2.09 (m, 1H), 1.26-1.23 (m, 9H). ¹³C NMR (100 MHZ, MeOD) δ 173.15, 171.35, 170.10, 169.93, 168.56, 166.85, 156.36, 147.45, 144.40, 136.18, 135.90, 131.66, 127.68, 126.76, 124.50, 123.05, 118.17, 116.50, 106.68, 71.23, 70.38, 69.87, 69.07, 64.94, 54.69, 49.21, 41.95, 39.12, 30.76, 28.78, 22.30. ESI-MS: [M+H]+ 789.15.

### Results and Discussion

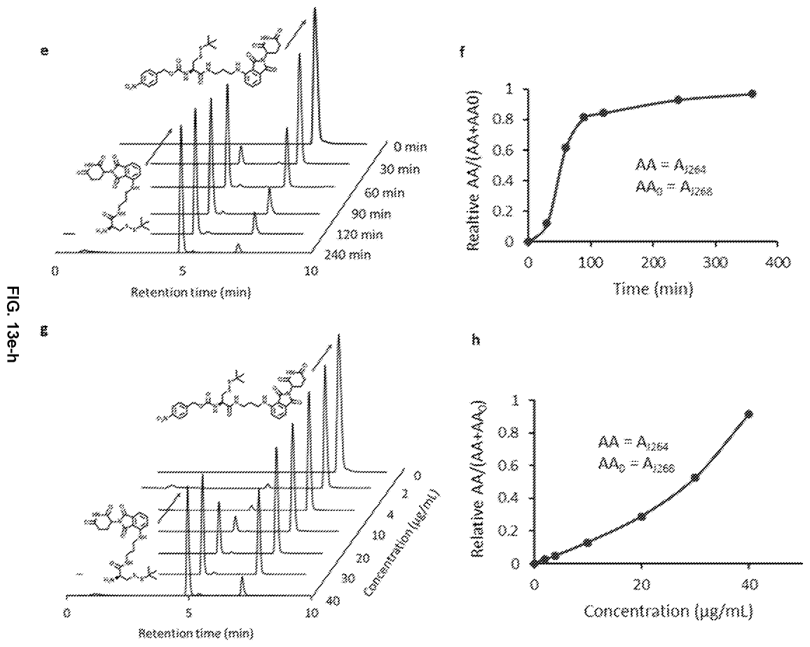

To achieve NTR-oriented ENCTAC, the rational design and molecular structures of different components, J266, JW4, JQ1-CBT and J252, were proposed in FIGS. 10 and 12a. Generally, the synthetic design is separated into two different parts including E3 ligase recruiting ligand (pomalidomide) and BRD4 targeting ligand (JQ1). For the pomalidomide-linked fragment, J266 was first synthesized through covalent coupling between pomalidomide-PEG₂-NH₂ and Boc-Cys(StBu)—OH. The thiol tert-butyl (-StBu) group serves as a GSH responsive moiety (FIG. 12a). Subsequently, the Boc-protected amino group was deprotected and further caged with p-nitrobenzyl chloroformate to obtain JW4, the molecular fragment that can both correspond to the NTR responsive uncaging and GSH reductive cleavage. On the other hand, JQ1 underwent esterification with 6-hydroxybenzothiazole-2-carbonitrile (CBT) to form JQ1-CBT. J252 was synthesized to serve as the standard PROTAC control molecule for preliminary analysis of the linker properties on the ENCTAC efficacy in targeted protein degradation.

### Example 10. Kinetics of ENCTAC Formation

Upon the exertion of external enzymatic stimuli, the NTR-responsive moiety undergoes self-immolation to unleash the amino group. Simultaneously, the presence of a reduction agent cleaves the -StBu group to liberate thiol group (—SH), creating the cleaved-J266. The presence of a readily reactive amino group and thiol group on cleaved-J266, with the co-insertion of JQ1-CBT would spontaneously induce the CBT-cysteine orthogonal click conjugation, shaping the luciferin-based structure in click-J252 for targeted protein disruption (FIG. 12a). To validate the kinetics of ENCTAC formation, JW4 was tested against the presence of ntr enzyme accompanied by the co-factor, NADH, at 37° C. across a range of time settings in LC-MS system. The formation of J252 in the presence of J266 and JQ1-CBT was also evaluated by LC-MS.

### NTR Enzymatic Cleavage In Vitro

### Procedure to Examine the Time Required for NTR to Perform Enzymatic Cleavage:

A MS vial was sequentially added JW4 (10 μM), nicotinamide adenine dinucleotide (NADH, 0.5 mM) and NTR (40 μg/mL), and the mixture was dissolved in PBS (pH 7.4, 10 mM) to a total volume of 500 μL. The mixture in the vial was vortexed and incubated at 37° C. with varying time control from 0-240 min. Structural mass analysis was performed to observe any molecular mass alteration.

### Procedure to Analyze the Optimal NTR Concentration:

A MS vial was sequentially added JW4 (10 μM) or J268 (10 M), NADH (0.5 mM) and NTR (concentration range of 0-40 μg/mL), and the mixture was dissolved in PBS (pH 7.4, 10 mM) to a final volume of 500 μL. The mixture in the vial was vortexed and incubated at 37° C. for 120 min before subjecting to structural mass analysis.

Selectivity of JW4 with or without NADH

A MS vial was sequentially added JW4 (10 μM), a reagent selected from sodium chloride, calcium chloride, ascorbic acid, glucose, cysteine, furin, CtsB, monoamine oxidase A (MAO-A) and monoamine oxidase B (MAO-B) (concentration range of 0-40 μg/mL), with or without the addition of NADH (0.5 mM) and the mixture was dissolved in PBS (pH 7.4, 10 mM) to a final volume of 500 μL. The mixture in the vial was vortexed and incubated at 37° C. for 120 min before it was subject to structural mass analysis.

### LC-MS Analysis of Click Reaction

2-mL MS vial was sequentially added J266 (10 μM), JQ1-CBT (10 μM) and lastly TCEP (200 μM), in a solution of pure DI H₂O to make a total volume of 500 μL. The compounds in the vial were vortexed for a few seconds to homogenize the mixture and was subsequently left to warm at 37-40° C. for 30, 60, 90 and 120 min to monitor the click reaction between cleaved-J266 and JQ1-CBT over time. A correlative line plot was performed to observe the kinetics and spontaneity of the click reaction.

### Results and Discussion

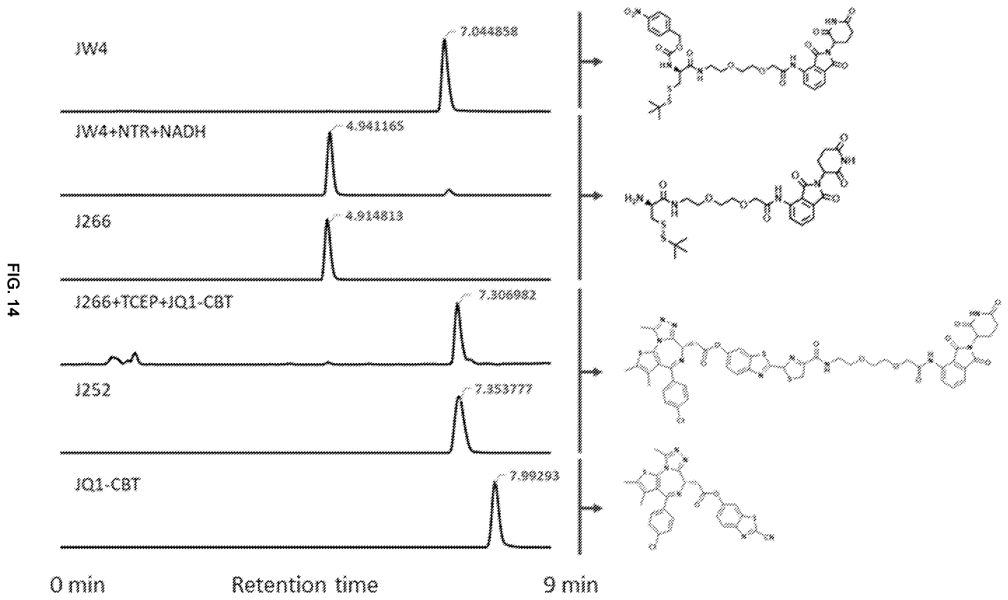

Over a span of 4 h under NTR enzymatic incubation, JW4 molecules were facilely self-immolated to J266 near to completion (FIGS. 12b and 13a-d) with the enzyme kinetics of Kₘ=101.29 μM and K_{cat}=0.0157 min-1. The experiments were repeated on J268 which has a disparate linker (C₃—NH₂ instead of PEG₂-NH₂), and the results indicate similar trend of enzymatic cleavage (FIG. 13e-h). These results reveal the flexibility of choosing a linker length and hydrophilicity in our NTR responsive ENCTAC system. In addition, to identify the specificity of NTR, a selectivity test of JW4 against different components and enzymes were performed, and JW4 showed no obvious cleavage except for NTR+NADH (FIG. 12c).

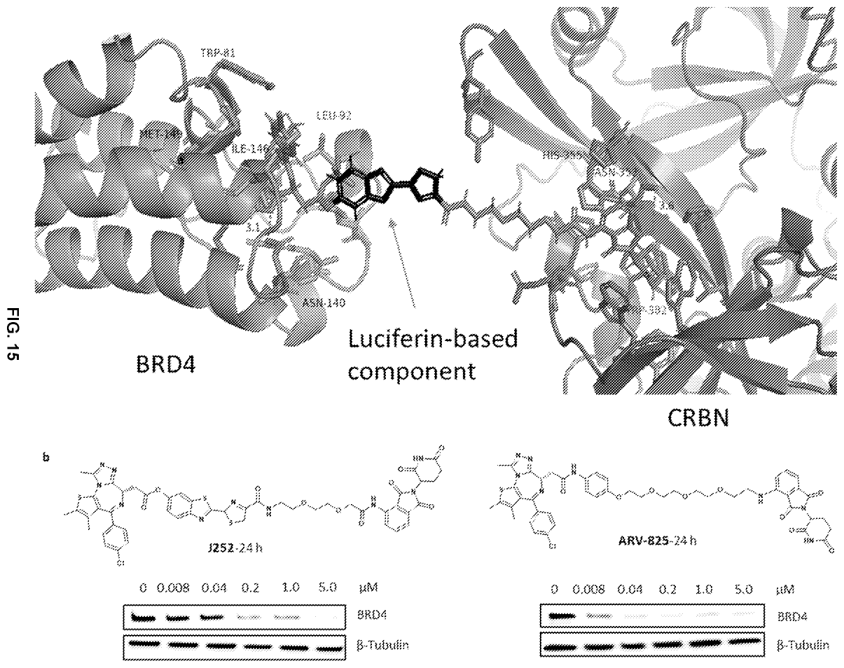

Furthermore, the formation of J252 in the presence of J266 and JQ1-CBT was also examined by LC-MS analysis (FIG. 12d-e). Upon the reduction of J266 by TCEP, the CBT-cysteine click formation occurred almost immediately after the -StBu cleavage of J266. The relative peak intensity of cleaved-J266 remained static but the peak intensity of click-J252 showed gradual increase, indicating the spontaneity and efficiency of the click conjugation. Importantly, the continuous enzymatic cleavage and click formation process were evaluated in one-pot reaction (FIG. 14), signifying the facile formation of the heterobifunctional degrader in a fixed enzymatic condition given the sufficient introduction of separated ENCTAC fragments.

### Example 11. Luciferin-Based Heterobifunctional Molecules Induce Degradation of BRD4

Inspired by the promising results in hypoxia NTR enzyme responsive ENCTAC conjugation, the molecular docking of click-J252 based on the crystal structures of BRD4 and CRBN (Winter, G. E. et al., Science 2015, 348, 1376-1381) was performed to assess the suitability of orthogonally cross-linking products in degrading protein of interest. Immunoblot analysis was performed by following the protocol in Example 12.

### Results and Discussion

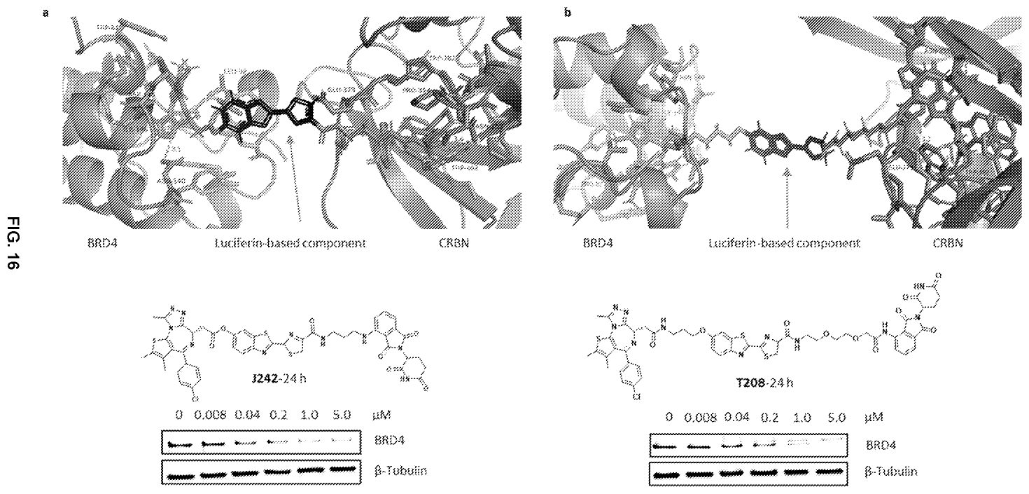

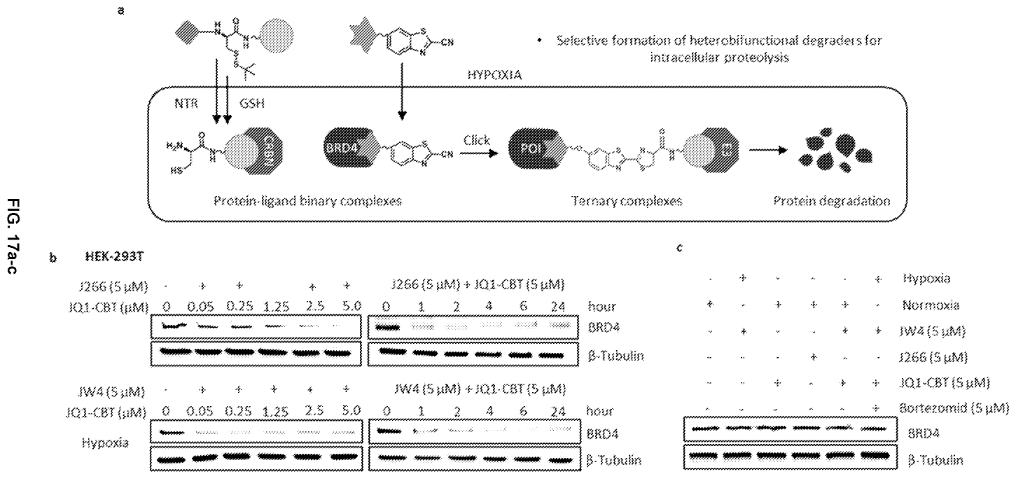

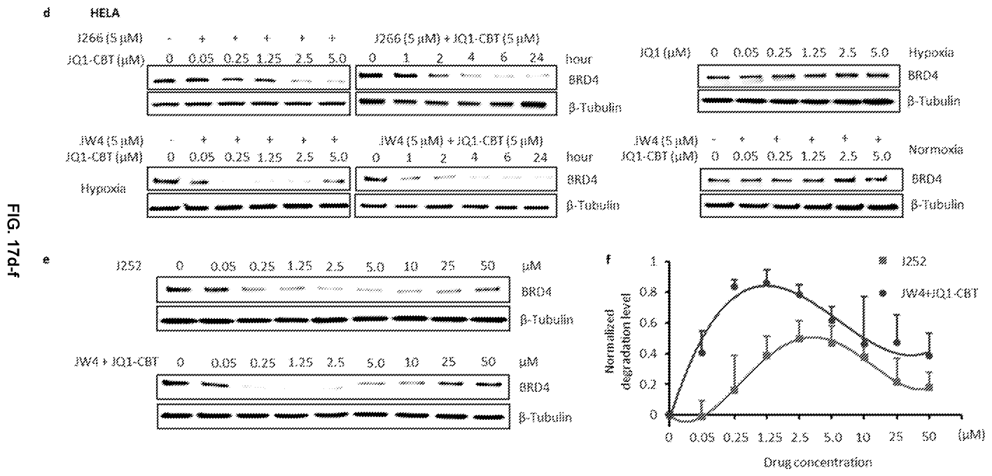

Simulation calculations revealed minimal steric hindrance in the pocket binding site of the two proteins with the luciferin-type linkers. In this context, the difference in linker lengths as indicated in the chemical structures of J252 and its analogues also shows trivial steric hindrance between the protein-ligand interaction (FIGS. 15a and 16a-b). Additionally, the non-hindered effect of the click product linkers in the PROTAC activity is further validated by the analysis of BRD4 protein level upon treatment with these molecularly-simulated compounds. Immunoblot analysis indicated that 24 h incubation of the PROTAC compounds induced concentration-dependent degradation of the BRD4 (FIG. 15b). The protein depletion effects were comparable with the previously reported ARV-825 degrader in the same cells (Lu, J. et al., Chem. Biol. 2015, 22, 755-763). These calculated and empirical evidences provided an insightful benchmark reference for our study, where we sought to use the ENCTAC for cell selective degradation of the epigenetic protein BRD4.

### Example 12. ENCTAC Facilitates Hypoxia Specific Degradation of BRD4

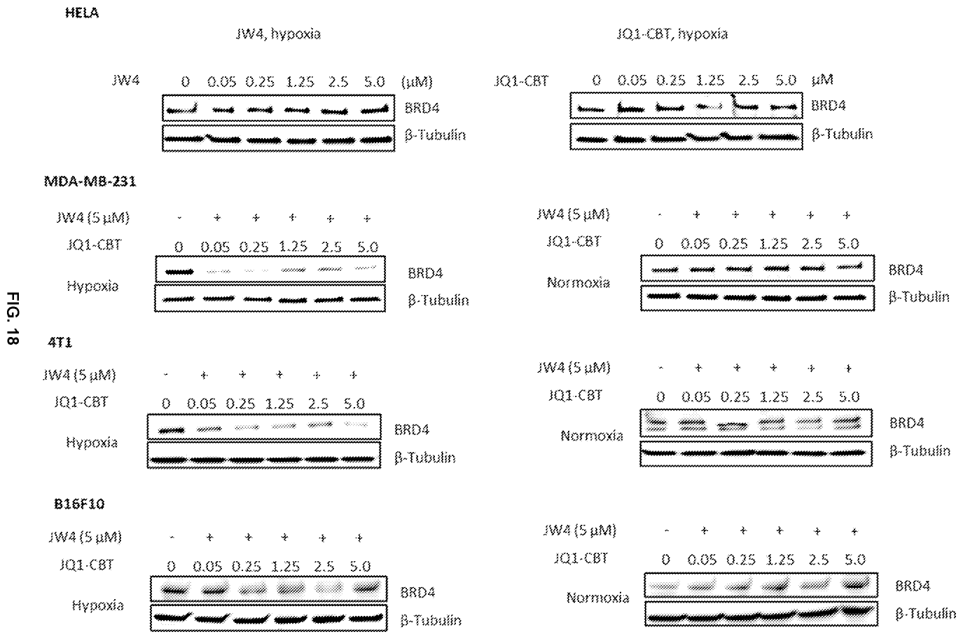

The effective production of BRD4 heterobifunctional degraders in solution allowed us to investigate the function of ENCTAC components in biological applications. Considering the upregulated NTR expression in oxygen-deficient cells, the hypoxia condition was constructed with the simultaneous incubation of the ENCTAC fragments (JW4 and JQ1-CBT). Particularly, NTR responsive JW4, the pomalidomide-containing compound that can target CRBN and is a universal E3 ligase, is expected to be uncaged through NTR-assisted nitro and GSH reduction once it enters the hypoxic cells. The exposed cysteine groups are then readily linked with the CBT moieties on the JQ1-CBT fragments via a specific bioorthogonal click reaction. Besides, the JQ1 warheads are presumed to be simultaneously bound to the BRD4 proteins, thus forming bifunctional degraders in-situ for localized proteolysis (FIG. 17a). Having demonstrated that the pre-synthesis click product (J252) offers great potential for BRD4 degradation, we sought to evaluate the viability of the method in selective proteolysis. As proof-of-concept, the control molecule J266 that can only respond to GSH but not NTR, was first introduced into the HEK293T cell line (for 6 h), with subsequent addition of the JQ1-CBT (for 12 h). After intracellular formation of the click-J252, the proteolysis process was observed by western blot analysis.

### Protein Degradation Analysis by Western Blot

HeLa, Hek293T, MDA-MB-231, 4T1 or B16F10 cells (1.5×10⁶ cells/mL) were treated with J266 (10 μM) for 6 h before incubating with JQ1-CBT at the indicated concentrations and time. This entire incubation was performed in normoxia condition. For NTR-moiety caged JW4, the cells were treated with JW4 (10 μM) 6 h prior to the addition of JQ1-CBT for a subsequent 12 h, and was performed entirely under hypoxic condition. Comparison of the downstream effect between the controls and drug-treated cells were completely executed in hypoxic condition. To work up, the cells were washed with cold PBS (pH 7.4) twice, and lysed with RIPA buffer supplemented with protease inhibitor cocktail and phosphatase inhibitor cocktail for 10 min at 4° C. Then, the lysed cells were collected and centrifuged at 13,500 rpm for 20 min at 4° C. The supernatant was collected and the protein concentration was analyzed by NanoDrop™ 2000/2000c Spectrophotometers. The lysate volumes were fine-tuned and loaded onto a Mini-PROTEAN® TGX™ 4-20% Precast Gel and was separated by SDS-PAGE. Then, the gel was transferred onto a PVDF membrane (100 V, 90 min, 4° C.), washed with TBS-T twice, and blocked with 1% BSA for 1 h at r.t. Next, the membrane was incubated with primary antibody overnight at 4° C., washed thrice with TBS-T, followed by incubation with goat anti-rabbit IgG (H+L) Secondary Antibody, HRP for 1 h. The membrane was supplemented with Radiance Q chemiluminescent ECL substrate and visualized using Amersham™ ImageQuant™ 800 biomolecular imager. B-Tubulin was used as the internal control. The involvement of proteasomal machinery was demonstrated by incubating the cells with proteasome inhibitor Bortezomib for 2 h before the addition of JW4 and JQ1-CBT.

### Results and Discussion

FIG. 17b shows that the protein degradation occurred in a concentration and time-dependent manner. The results from this exclusively GSH responsive uncaging molecule of J266 illustrate the possibility of our ENCTAC to facilitate proteolysis after conjugation of two separated fragments. We further examined the hypothesis of cell selectivity activation of small molecule induced protein degradation by using the NTR-responsive compound, JW4. The BRD4 protein level in the HEK293T cells that were subject to hypoxic condition showed significant reduction with increased concentration of ENCTAC compounds. Similar to J266, the proteolysis process occurred at the early state after the introduction of JW4 and JQ1-CBT. After 24 h, most of the BRD4 was degraded with 5 μM of the test compounds. The control experiments were also processed, and the results showed that independent treatment of the cells with JW4, JQ1-CBT, and J266 alone for 12 h did not induce any degradation of the BRD4 (FIG. 17c). Notably, the pre-treatment of the cells with Bortezomib before adding the NTR-activated ENCTACs showed trivial BRD4 degradation. Such interference demonstrated the involvement of proteasomal machinery in our ENCTAC system.

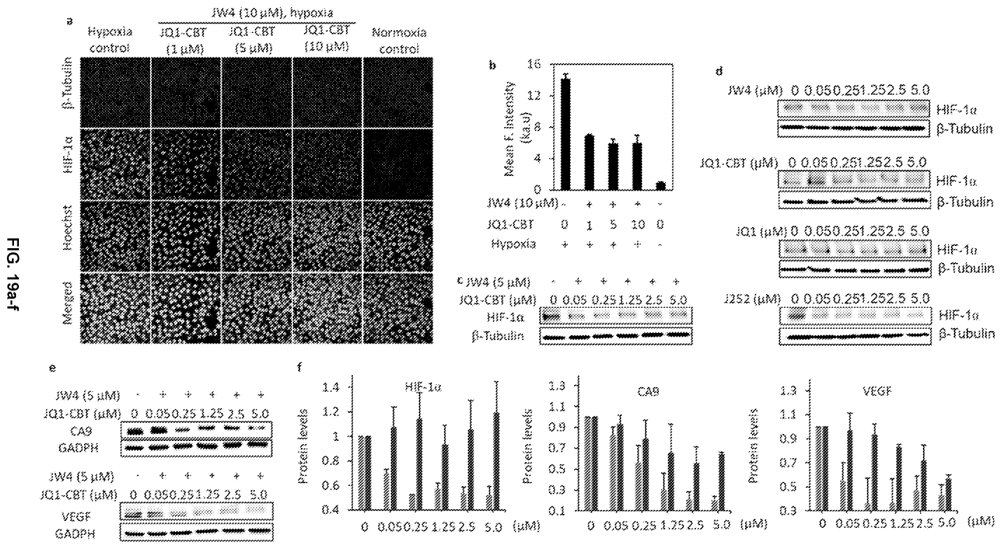

The selective BRD4 protein degradation was further examined in different tumour cells including HeLa, MDA-MD-231, 4T1 and B16F10 melanoma cells. Immunoblots revealed effective decrease in BRD4 levels as compared to the untreated cells in hypoxia environment with molecule concentration and time dependence (FIGS. 17d and 18). In contrast, distinct treatment of hypoxic cells with the sole fragmented ENCTAC molecules did not implicate degradation of the targeted protein levels. Similar result was also observed in the Hela cells treated with (+)-JQ1 (BRD4 inhibitor) in hypoxic condition. Likewise, co-incubation of the ENCTAC molecules did not trigger protein degradation in the multiple cell lines under normoxia environment (FIG. 18). This observation clearly indicates that co-presence of both fragments is required for the activation of degradation processes, and indeed, the NTR-activated ENCTAC is specific in hypoxic cells.

Notably, in conventional PROTAC systems, the “hook effect” is omnipresent in the condition of excessive introduction of heterobifunctional degraders (Burslem, G. M. & Crews, C. M., Cell 2020, 181, 102-114). This effect is expected to be naturally-occurring across all ternary complexes in which three components are combined to induce activities (Douglass, E. F. Jr. et al., J. Am. Chem. Soc. 2013, 135, 6092-6099). In conventional PROTAC systems, saturated amount of the degraders will effectuate in excessive binary complexes, thus minimizing the amount of active ternary complexes and degradation of targeted protein. Our J252 compound showed “hook effect” like conventional PROTACs, when high concentration of the compound was used (FIG. 17e). In this case, the decrease in the efficiency of the proteolysis was started to be observed on 5 μM of J252, and was markedly interfered by 50 μM of the compound with the degradation level of only around 20%. In our ENCTAC system with NTR responsive JW4 and JQ1-CBT, this effect was also observed. This effect indicated the necessity of forming ternary complexes via the recruitment of the E3 ligase to the BRD4 by the click reaction. However, the proteolysis process was obtained at lower concentrations of the ENCTACs but higher protein degradation efficacy in comparison with the whole molecule J252 treatment, possibly due to the better intracellular uptake of the compounds (FIG. 17f). Although “hook effect” occurs innately, the lower concentration effect featured in our ENCTACs offers a promising opportunity to enhance the application of small molecule targeted protein degradation, in which lower molecular weight, simpler chemical preparation and better cellular penetration of the compound can be achieved with enhanced site-specificity and pharmacological performance.

### Example 13. ENCTAC Deactivates Hypoxia Response Via Suppression of HIF-1α

Epigenetic regulators are essential for HIF-mediated trans-activations. In particular, BRD4 is one of the epigenetic readers that is recruited by ZMYND8 to the hypoxia-responsive elements (HREs) of the HIF target genes. This protein interacts with positive transcription elongation factor b (P-TEFb) and supports the HIF activation mediating release of paused RNA polymerase II and elongation of the HIF target genes (Chen, Y. et al., J. Clin. Invest. 2018, 128, 1937-1955; Jang, M. K. et al., Mol. Cell 2005, 19, 523-534; and Galbraith, M. D. et al., Cell 2013, 153, 1327-1339). So far, the first generation BET bromodomain inhibitors which are JQ1-related compounds were tested in the clinics but showed a relatively low potency (Doroshow, D. B., Eder, J. P. & LoRusso, P. M., Ann. Oncol. 2017, 28, 1776-1787). Moreover, JQ1 and its analogues have shown their inability to suppress HIF-1α albeit the reduced expression of hypoxia-regulated genes was observed, possibly because of its non-specific binding effects (da Motta, L. et al., Oncogene 2017, 36, 122-132; and Yin, M. et al., Nat. Commun. 2020, 11, 1833). In contrast, our NTR-activated ENCTAC system specifically degrades BRD4 and therefore, potently deplete its interactions and functions with other co-functional proteins.

### Immunofluorescence Imaging of HIF-1α

Hela cells were plated at a density of 8×10⁴ cells/mL in each well of the eight-well ibidi dishes before experiment. To sustain the hypoxia-induced factor throughout the drug treatment process, drug incubation was performed entirely in hypoxic chamber. Initially, the Hela cells were treated with JW4 (10 μM) for 6 h. After that, JQ1-CBT (10 μM, 5 μM or 1 μM) were added into the respective wells and incubated for an additional 12 h. The control cells were left untreated under normoxia condition on a separate ibidi dish. The cells were fixed with 4% paraformaldehyde for 15 min, permeabilized with 0.25% TBS-T, and blocked with 1% BSA for 20 min at r.t. post-drug treatment. All the different cell groups were then incubated with rabbit HIF-1α polyclonal antibody for 2 h at r.t. Then, the primary antibody-labelled cells were rinsed with PBS (2×), and stained with Alexa Fluor® 488-goat anti-rabbit IgG secondary antibody for 1 h at r.t. The cytoskeleton networks of the cells were stained with Alexa Fluor® 594 Anti-α Tubulin antibody [DM1A]—Microtubule Marker, and the nuclei were stained with Hoechst 33258 (1 μM). The fluorescence of the alleviated HIF-1α expression upon drug treatment was visualized with Carl Zeiss LSM 800 microscopes (Hoechst 33258: Ex=405 nm, Em=460/50 nm; Alexa Fluor 488: Ex=488 nm, Em=515/30 nm; Alexa Fluor 594: Ex=561 nm, Em=617/20 nm).

### Apoptosis Imaging and Flow Cytometry (FCM) Analysis

Dual staining molecular probe containing AnnV and PI was used to invigilate the apoptotic condition post-drug treatment. Generally, drug-treated cells were incubated with 5 μL of AnnV in 100 μL of 1× binding buffer for 15 min at r.t. in the dark. The staining medium was removed and without further washing, 1 μL of PI staining solution in 100 μL of 1× binding buffer was added to the Hela cells for another 15 min at r.t. The means of visualization were performed by Carl Zeiss LSM 800 confocal laser microscope (FITC-AnnV: Ex=488 nm, Em=520/21 nm; PI: Ex=561, Em=590/30 nm) for apoptosis imaging, or by BD LSRFortessa X-20 flow cytometer for quantification of AnnV-PI staining in different group of cells, control and post treatment.

### Cell Viability Assay

Hela cells were seeded at a density of 1×10⁴ cells/mL with a total volume of 100 μL DMEM in 96-well plate overnight. Subsequently, pre-set JW4 concentration at 10 μM was incubated under hypoxic condition for 6 h prior to the addition of JQ1-CBT at varying concentrations for 24 h in hypoxia before replacement with TOX-8 medium solution (0.3 mg/mL), followed by incubation for another 4 h. Cell viability analysis was then analyzed by Tecan Infinite M200 microplate reader with the excitation at 560 nm and emission at 590 nm. The percentile of cell viability was obtained by the ratio of the fluorescence intensity of drug-treated cells relative to the untreated control cells.

### Results and Discussion

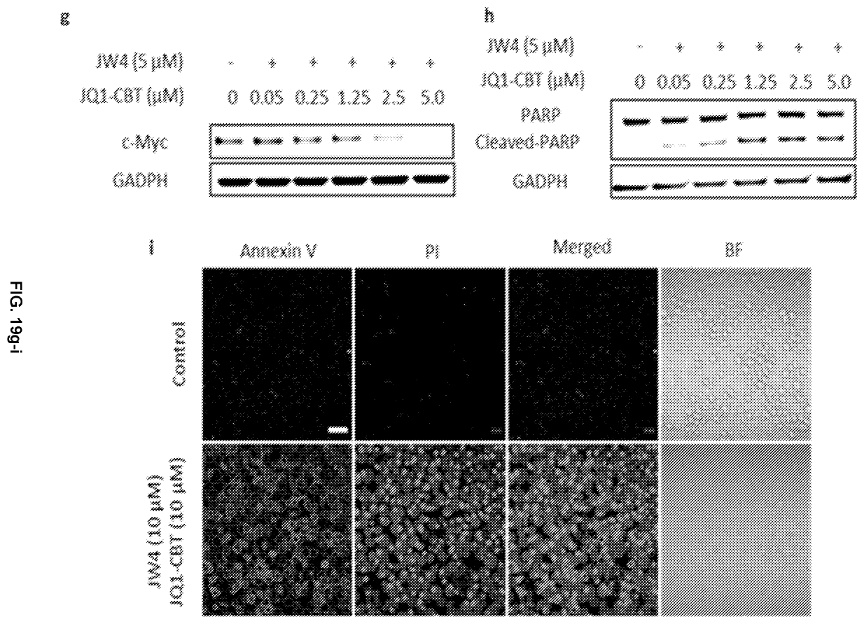

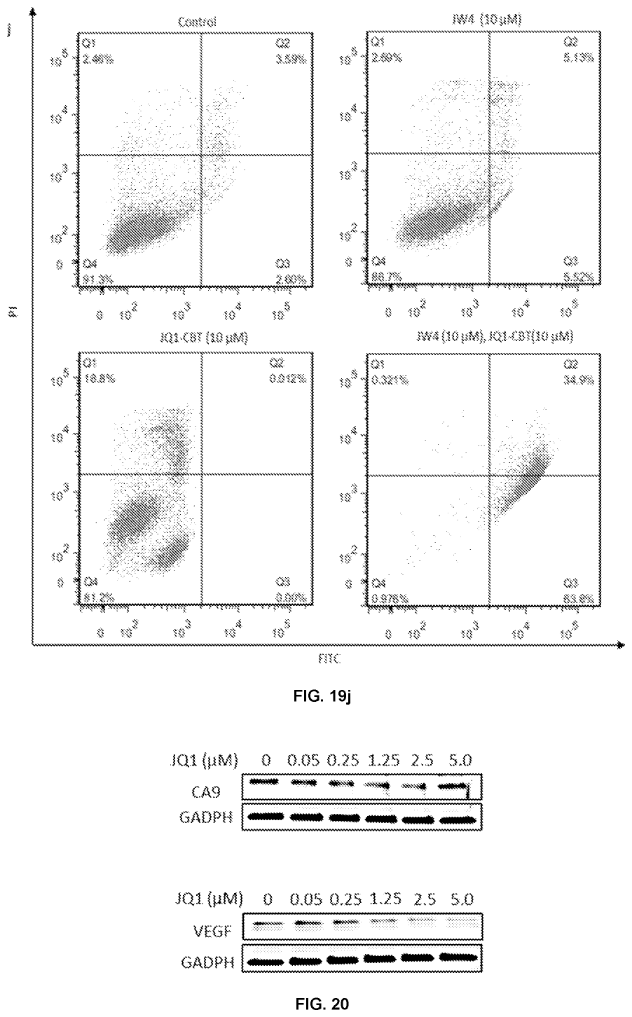

The diminished activity of these epigenetic proteins directly consequences in the minimizing of the expression of HIF-1α, as indicated in FIG. 19a-b. Immunostaining was managed with green-fluorescent labelling of the HIF-1α in accordance with nucleus and skeleton protein, β-tubulin. Microscopy images illustrated the nucleus localization of HIF-1α during hypoxia, which is associated with the hypoxia gene mediation processes. The level of this protein was reduced upon treatment with our ENCTAC compounds (JW4 and JQ1-CBT), which was also observed in the western blot analysis (FIG. 19c). Similarly, J252 (the standard PROTAC compound) was able to downregulate the expression of HIF-1 in hypoxic Hela cells. In contrast, separated incubation of JW4 and JQ1-CBT under hypoxia did not induce HIF-1α elimination (FIG. 19d), which is consistent with the static protein level of BRD4 (FIG. 18). Meanwhile, the inhibition of BRD4 with JQ1 alone in hypoxia did not alter the protein level of HIF-1α in Hela cells. These results unequivocally illustrate that the degradation of BRD4 interrupts the expression of HIF-1α in the nucleus. In addition, as compared to the standard inhibition, our NTR-activated ENCTAC systems indicate different perturbation pathway to manipulate the hypoxia response.

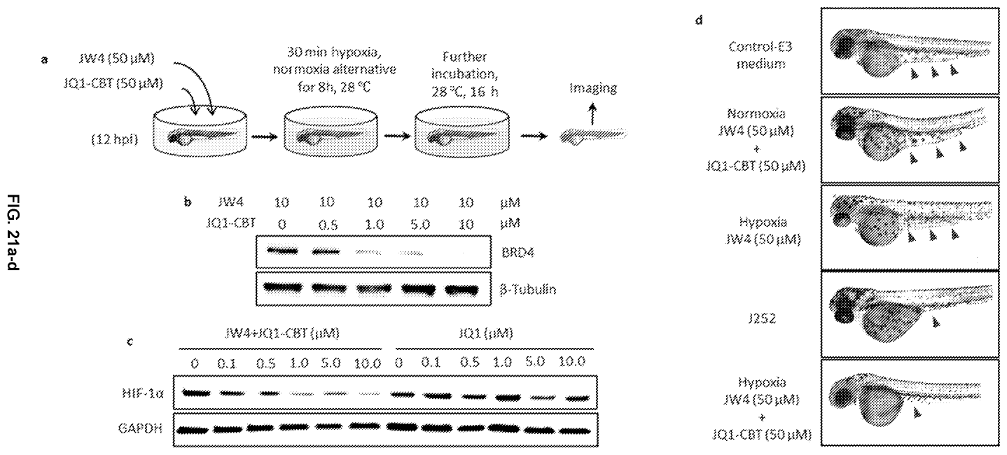

To further investigate the effects of ENCTAC degradation of BET proteins on hypoxia-regulated pathways, we evaluated expressions of the hypoxia-responsive proteins. Particularly, CA9 which is a cellular biomarker and pH regulator of hypoxia, is overexpressed in cells in abnormal vasculature and tumour microenvironment (Haapasalo J. A. et al., Clin. Cancer. Res. 2006, 12, 473-477). This protein is transcriptionally activated by HIF-1α binding. JW4 and JQ1-CBT co-incubation under hypoxia dose-dependently reduced the amount of CA9 as a consequence of BRD4 and HIF-1α downgrade (FIG. 19e). Although JQ1 treatment did not suppress BRD4 and HIF-1 protein levels, it can still partially downgrade CA9 expression (FIG. 20). Such minimized protein expression can be possibly attributed to the inhibition of BRD4 and CA9 gene promoter interactions (da Motta, L. et al., Oncogene 2017, 36, 122-132). Additionally, similar effects were also observed in the blood vessel stimulated protein, VEGF (FIGS. 19e and 20). This protein restores the oxygen supply when blood circulation is inadequate such as those in hypoxia (Forsythe, J. A. et al., Mol. Cell. Biol. 1996, 16, 4604-4613). Quantitative analysis of protein levels after ENCTAC or inhibitor treatments revealed the significant difference in the impact of BRD4 degradation on HIF-1α expression levels and its hypoxia-regulated proteins (FIG. 19f). Specifically, almost 50% lower in HIF-1α level was observed in cells with ENCTAC treatment in contrast to the static HIF-1α level done by JQ1 incubation at the same concentration, suggesting that degradation of BRD4 by ENCTAC provided a more efficient down-regulation of CA9 and VEGF proteins. Such combined perturbation of CA9 and VEGF triggered by specific ENCTAC degradation may provide the possibility to enhance sensitivity towards tumour therapies.

The ENCTAC treatments were also investigated in tumour cells under hypoxia settings. Importantly, the targeting BRD4 degradation significantly induces cell development malfunctions. Once the adaptive system of microenvironment alterations is interfered by HIF-1a deductions, the cells may undergo apoptosis and are thus unable to sustain the development process (Loncaster, J. A. et al., Cancer Res. 2001, 61, 6394-6399; and Pantuck, A. J., Clin. Cancer Res. 2003, 9, 4641-4652). To this end, we analysed the typical cell growth factor c-Myc, and indeed, reduced amount was observed after NTR-oriented ENCTAC introduction (FIG. 19g). In the meantime, the cleavage of PARP was increased, signalling the association of apoptosis (FIG. 19h). The apoptotic response was further detected by Annexin V/PI staining. Confocal microscope images revealed the clear Annexin V translocation and the surge in cell membrane permeability after drugs introduction for 12 h (FIG. 19i). Quantification of the staining signals were recorded by flow cytometry (FIG. 19j), illustrating an obvious late apoptotic cell death after BET proteolysis in hypoxia. These results clearly indicate the potency of microenvironment specific BET degradation for game-changing consideration of therapeutic applications.

Taken together, notably, after hypoxia stress stimulation, the immunoblot analysis clearly demonstrated a significant decrease of BRD4 level in multiple hypoxic cells across ascending concentrations of the small-molecule substrates. Along with the disruption of BRD4 expression, the obvious proliferation suppression was observed in the tumour cells, suggesting the feasibility of ENCTAC for cancer inhibition.

### Example 14. In Vivo ENCTAC Dependent Angiogenesis

We next extended our analysis of hypoxia activated ENCTAC to ascertain its effect in vivo using zebrafish larvae. The relatively high level of BRD4 and the conserved structure similarity of CRBN and JQ1 binding site between zebrafish and human make it an ideal predictive model for protein-ligand activity in higher organism including human (Toyama, R., Dev. Dyn. 2008, 237, 1636; and Gang, X. et al., J. Am. Chem. Soc. 2019, 141, 18370-18374).

### Zebrafish Experiments

Briefly, early embryogenesis zebrafish was first used to evaluate the in vivo activity of ENCTAC. 12 hpf zebrafish was incubated with hypoxia responsive ENCTAC compounds at 50 μM in a hypoxia-normoxia alternating condition for 8 h at 28° C. Embryos without drug treatment, ENCTAC system in normoxia condition, and treatment with only JW4 (the pomalidomide-containing moiety) in hypoxia were used as the negative controls, and embryos treated with the standard PROTAC compound J252 was used as the positive control. Whole extract of embryo bodies after treatment was collected and analysed by immunoblotting.

Wide type zebrafish embryos at 12 hpf was incubated with J252 (50 μM), JW4 (50 μM), or the mixture of JW4 (50 μM) and JQ1-CBT (50 μM) in hypoxia-normoxia alternative condition for 8 h at 28° C. After that, the embryos were further incubated in normoxia condition at 28° C. for 16 h before the yolk extension phenotypes were visualized under Zeiss LSM800 confocal microscope using bright-field channel. Embryos with JW4+JQ1-CBT treatment under normoxia were used as the controls.

For protein degradation analysis of zebrafish, JW4 and JQ1-CBT mixture or JQ1 in different concentrations was incubated with the zebrafish embryos at 24 hpf for 8 h with 30 min alternating intervals of hypoxia-normoxia condition. Embryos without drug treatment were used as the controls. Subsequently, the lysis of the embryos was collected and the BRD4 and HIF-1α protein levels were analysed by immunoblotting method by following the protocol in Example 12.

### Confocal Imaging

Vasculature zebrafishes were obtained from natural crosses of the AB wild-type strain or vhl^{+/hu2117} carriers on the fli1a:egfp^{y1} transgenic background (which fluorescently marks the vasculature). JW4 and JQ1-CBT were dissolved in DMSO and the mixture was applied to embryos at equimolar concentration of 10 μM, with J252 (10 μM) and JQ1 (10 μM) used for the control experiments. VHL^{-/-} of transgenic zebrafish or wide type zebrafish after treatment with JW40 (50 μM) for 12 h was also taken for confocal imaging. The vascular plexus was imaged on a Zeiss LSM800 confocal microscope. Green: blood vessel tracker, .ex: 488 nm, .em: 520/30 nm; red: . ex: 564 nm, .em: 610/30 nm.

### Results and Discussion

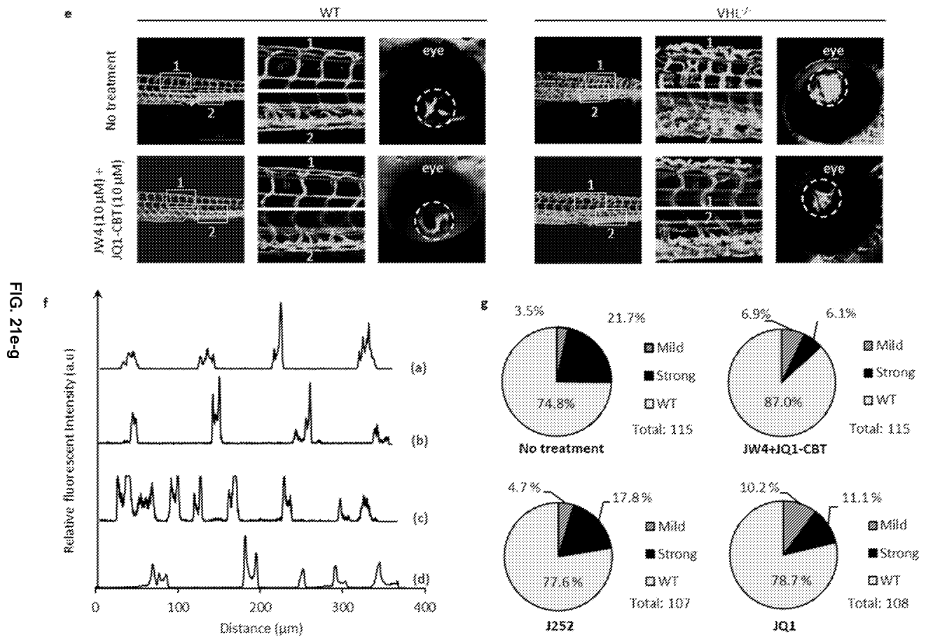

As shown in FIG. 21b, our ENCTAC system in hypoxia condition significantly reduced the level of BRD4 protein in embryos. Such hypoxia-mediated BRD4 degradation also resulted in lower expression level of HIF-1α in hypoxic zebrafish embryos which contrasts with the epigenetic inhibition of BRD4 with JQ1 itself (FIG. 21c). Phenotype observation after treatment illustrated that the embryos incubated with hypoxic ENCTAC system showed thinner yolk extension compared to those of controls. Embryos treated with directly linked PROTAC control molecule J252 showed a similarly less extension of yolk at 36 hpf (FIG. 21d). These results demonstrate the selective degradation of the targeted protein in zebrafish.

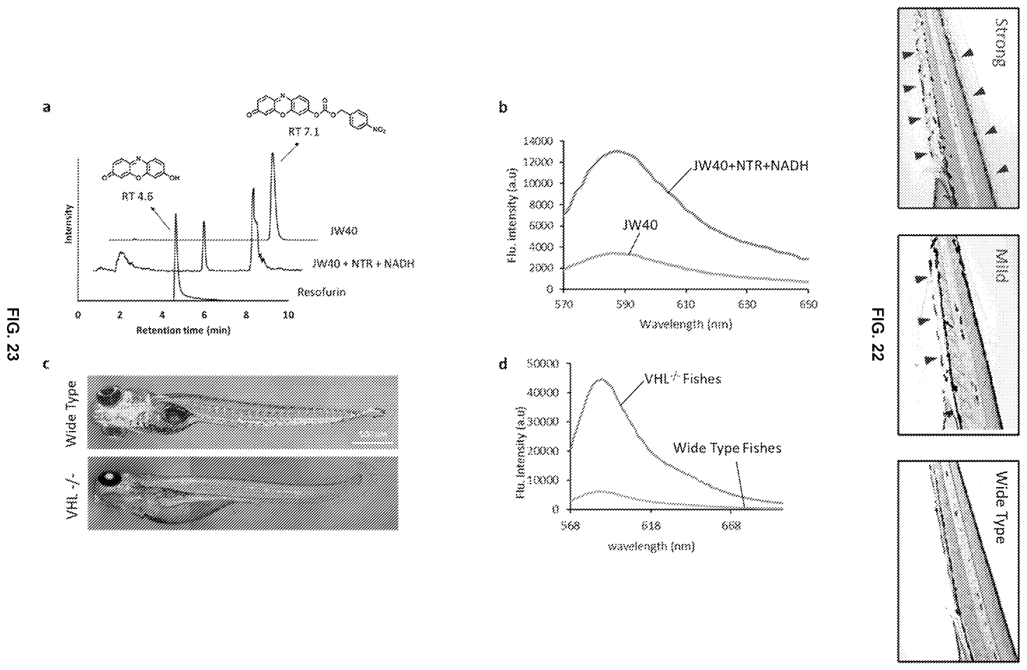

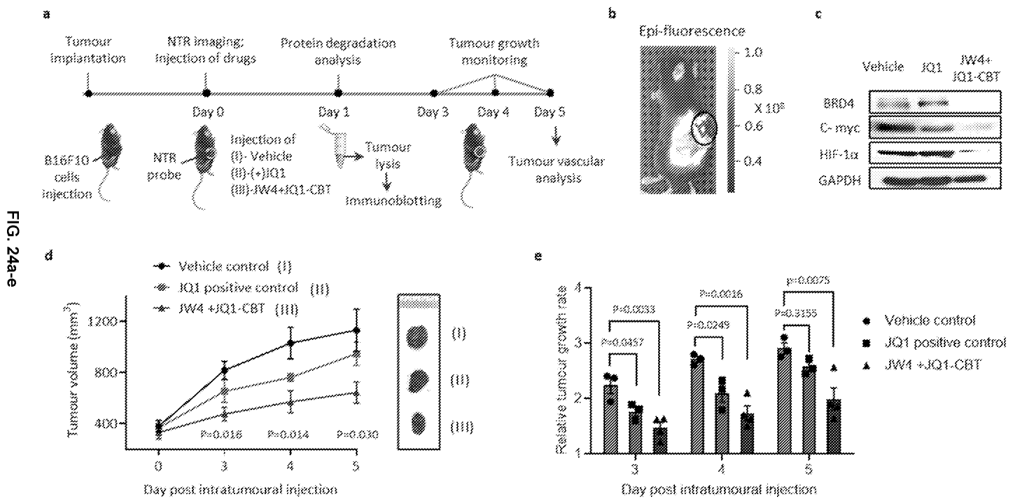

In line with the efficiency of our system in downregulating the hypoxia responsive proteins including VEGF in cancer cells, and its ability to reduce HIF-1α level via BRD4 depletion in zebrafish, we next evaluated the ENCTAC capacity in modulating angiogenesis which is one typical hallmark in tumours. We utilized vhl^{hu2117} mutant larvae, which lacks a negative regulator of HIF-1α (VHL), to comprehend the direct relationship between BRD4 and HIF-1α (Bousquet, M. S., ACS Chem. Biol. 2016, 11, 1322-1331). The excessive level of HIF-1α in the larvae resulted in extensive vascularization due to the upregulation of VEGFA (FIG. 22). The mutant larvaes showed NTR activation as detected by our NTR-responsive dye in comparison with wide type larvae (FIG. 23). VHL mutant larvae showed significant enhancement in red fluorescence from NTR uncaging of JW40. While the ENCTAC compounds showed negligible effect on wide type vascular patterning, they reduced the diameter and total length of the intersomitic vessels in the tail plexus and retina of the mutants (FIG. 21e-f). Statistically, the ENCTAC systems significantly decreased the number of vascularized larvae (13%) compared to the non-treatment group with approximately one quarter of vhl^{hu2117} homozygous mutants. Meanwhile, treatment of the transgenic larvae with the standard J252 or JQ1 alone slightly lowered the severity of vascular phenotype with 22.5% and 21.3% vascularized larvae remaining, respectively (FIG. 21g). Such selective suppression of the severe vascular phenotype further illustrates the promising effect on anti-angiogenesis, and the superiority of our ENCTAC system to the activity of conventional PROTAC or inhibitor compounds in vivo.

### Example 15. ENCTAC Activity in Solid Tumours

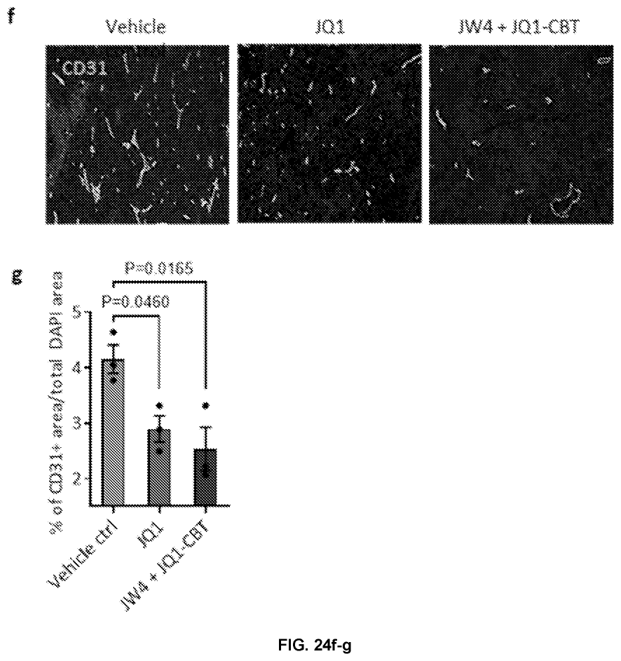

We further investigated the feasibility of tumour microenvironment-activatable epigenetic BRD4 protein degradation and further validated the potential anti-tumour efficacy of ENCTAC in a C57BL/6J mice model. Typically, we implanted B16F10 mouse melanoma cell line into the right flank of living mice and the solid tumour was monitored to grow until palpable size before undergoing targeted protein disruption and therapeutic studies (FIG. 24a). The living mice bearing tumours were first subject to NTR-Cy7, a reported NTR enzyme responsive probe molecule (Li, Y. et al., J. Am. Chem. Soc. 2015, 137, 6407-6716), followed by animal imaging scanning.

### Protein Degradation Study in Tumour Mouse Model

Cells were cultured in DMEM supplemented with 10% FBS, 2 mM of I-glutamine, 100 U/mL of penicillin, and 100 μg/mL of streptomycin. The cell line was maintained at 37° C. in a humidified atmosphere of 95% air and 5% CO₂.

B16F10 cells at a density of 2×10⁶ cells were injected subcutaneously into the right flank of six- to eight-week-old mice. Once the tumours became palpable, intratumoral injection of the buffer solution as vehicle, JQ1, and JW4 and JQ1-CBT at dosage of 5 mg/kg were performed for every 4 h up to 8 h. The mice were sacrificed and protein extraction were carried out. The protein levels in lysis samples were analysed by western blot by following the protocol in Example 12. Histology and immunofluorescence staining were performed by following the protocol in Example 13. Specifically, the resected tumours were fixed in 4% paraformaldehyde for 48 h, washed with PBS, and gradually transferred to 15% sucrose, followed by 30% sucrose before being embedded in O.C.T. compound. Five-micrometre cryosections of the tumour samples were dehydrated and blocked with a blocking buffer containing 2% BSA, 1% Tween 20, 3% Triton X, and horse serum for an hour before being incubated with primary antibodies, followed by a washing step and then incubation with secondary antibodies. The primary antibodies used were Ki67 and CD31. The secondary antibodies employed were goat anti-rabbit IgG (H+L) cross-adsorbed secondary antibody, Alexa Fluor 594, and goat anti-rat IgG (H+L) cross-adsorbed secondary antibody, Alexa Fluor 488. The nuclei were counterstained with DAPI. The slides were subsequently washed with PBS, mounted with Mowiol, and visualised under Leica TCS SP8 Confocal and STED 3× inverted confocal microscope. Images were captured using the EVOS M5000 imaging system (Thermo Fischer Scientific, Waltham, MA, USA) and quantified using ImageJ.

### Results and Discussion

As shown in FIG. 24b, the solid tumour in living mice indicated the hypoxia environment and activation of NTR in tumour model. Inspired by the promising imaging results, we further accessed the acute pharmacodynamic degradation of BRD4. In this study, tumour bearing mice were treated with intratumoral injection of ENCTAC, JW4+JQ1-CBT, at dosage of 5 mg/kg for every 4 h up to 8 h at day 1 of the treatment. Meanwhile, buffer solution as the vehicle and JQ1 (standard BRD4 inhibitor) were used as controls for the tumour responsive protein degradation and therapeutic studies. As expected, administration of ENCTAC compounds (JW4+JQ1-CBT) demonstrated obvious BRD4 degradation. Accompanied investigations disclosed the downregulation of HIF-1α and c-Myc (FIG. 24c), suggesting the close correlation of epigenetic regulation to the tumour hypoxia. Although the injection of JQ1 alone reduced tumour growth and downgrade c-Myc expression in xenograft model, unlike our ENCTAC compounds, such epigenetic BET inhibition had trivial effect to alter BRD4 and HIF-1a protein levels, indicating their different mechanism to impair tumour response to hypoxia. The antitumour efficacy of our ENCTAC system was evaluated by daily treatment of the compounds at equimolar concentrations. Serial volumetric measurement illustrated significant attenuation of tumour progression utilizing ENCTAC as compared to vehicle controls and JQ1 administrations (FIG. 24d), and decreased tumour growth rates were also observed (FIG. 24e).

To compare the efficacy of BET inhibition to BET degradation in tumour vascularization, we use a standard blood vessel marker, CD31. Our immunofluorescence staining of the excised tumours at the end of 5th-day efficacy study suggested a significant inhibition of CD31 in comparison with vehicle controls and JQ1 (FIG. 24f-g). This indicates the better effect of our ENCTAC degradation on the tumour hallmark, angiogenesis. Indeed, the antitumour efficacy of our ENCTAC system was further evaluated by daily treatment of the compounds at equimolar concentrations. Serial volumetric measurement clearly illustrated significant attenuation of tumour progression utilizing ENCTAC as compared to vehicle controls and JQ1 administrations (FIG. 24c), and decreased tumour growth rates was also observed (FIG. 24d). These findings prove an improved antitumour efficacy in mouse xenograft using hypoxia activated ENCTACs.

Importantly, although our in vitro and in vivo investigations demonstrated that the epigenetic BRD4 substrate JQ1 alone could indeed function as BET inhibitor and impair the CA9 and VEGF stimulation induced by hypoxia, such occupancy driven BRD4 inhibition non-specifically occurred in both hypoxia and normoxia, and the epigenetic protein inhibition did not alter the cellular HIF expression. Similarly, the direct cross-linking of pomalidomide and JQ1 substrate, as a control similar to that of traditional PROTAC design, could indeed disrupt epigenetic BRD4 in live cells, but there was no selectivity to differentiate between normoxia and hypoxia cells. Therefore, it could implicate detrimental off-target effect. Moreover, the large molecular weight of conventional PROTAC molecule also raises the concern of poor pharmacological performance (such as cell permeability and efficient protein perturbation). The fragmented ENCTAC strategy and the in-situ formation of ENCTAC upon site-selective activation could ideally resolve these issues, and our results demonstrate that our ENCTAC strategy could indeed realize environment-specific PROTAC formation and targeted protein degradation without the need to tediously link two protein substrates in advance, thereby evading the sophisticated synthetic method as generally seen in conventional PROTAC designs. In addition, a significant decrease in HIF expression was observed in hypoxic cells and animals, suggesting the key roles of epigenetic regulator to mediate HIF transcription in hypoxia and its different signalling pathway involved as compared to the physical target suppression manipulated by simple molecular inhibitor like JQ1. Such environment-responsive HIF perturbation demonstrated obvious anti-angiogenic effect that could significantly reduce vascularization in hypoxic zebrafish models and solid tumours. Such superior ENCTAC strategy can also be easily extended to other tumour environment responsive systems which arm the PROTAC technology with more compelling practicality and druggable potency to address their full therapeutic potential in the near future.

1. A compound of formula Ia: 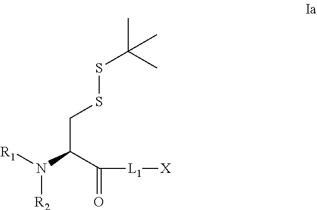
where:
R₁ is H or one or more amino acids selected from Y, A, W, H, L, Q, T, D, E, and more particularly R, V, F, and K, where a terminal amino acid of the one or more amino acids has a free NH₂ group or a NHAc group;
R₂ is H or 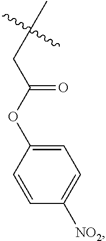 [SMILES: *CC(=O)Oc1ccc([N+](=O)[O-])cc1]
where the wiggly line represents the point of attachment to the rest of the molecule;
L₁ is a linking group; and
X is an E3 ligase ligand or a protein of interest ligand,
or a pharmaceutically acceptable salt or solvate thereof, provided that at least one of R₁ and R₂ is not H.

2. The compound according to claim 1, wherein R₁ is selected from the group consisting of AcDEVD-, AcYVAD-, AcWEHD-, AcVDVAD-, AcLEVD-, AcLEHD-, AcVQVD-, AcLETD-, DEVD-, YVAD-, WEHD-, VDVAD-, LEVD-, LEHD-, VQVD-, LETD-, and more particularly H, RVRR- or Ac-FK-.

3. The compound according to claim 1, wherein when L₁ is attached to a nitrogen atom on X, it is selected from: a bond; or 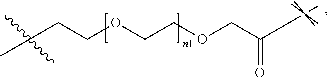
where the wiggly line represents the point of attachment to the carbonyl group next to L₁ and the crossed line represents the point of attachment to X and n1 represents from 0 to 5; or 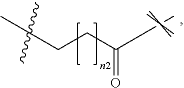
where the wiggly line represents the point of attachment to the carbonyl group next to L₁ and the crossed line represents the point of attachment to X and n2 represents from 0 to 10.

4. The compound according to claim 3, wherein X is selected from: 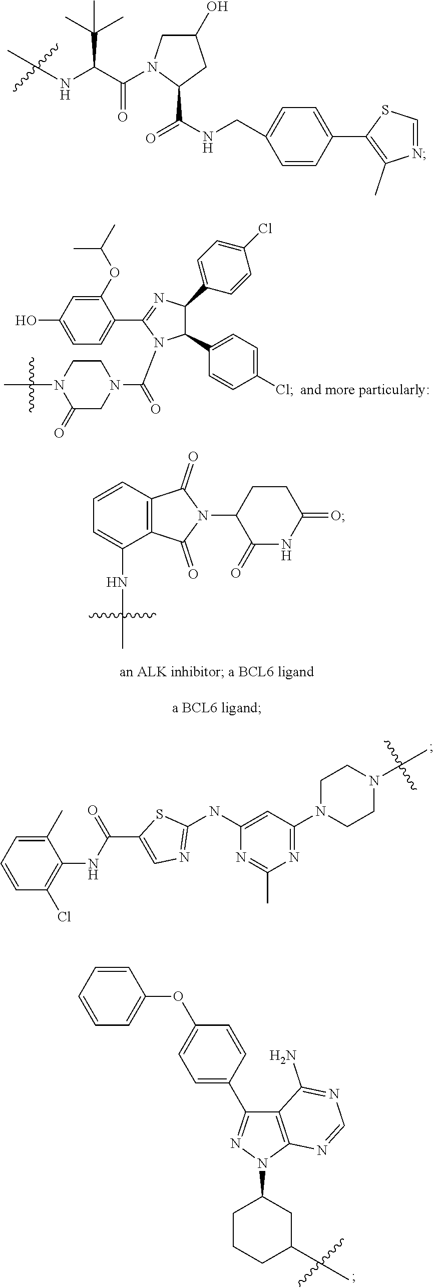 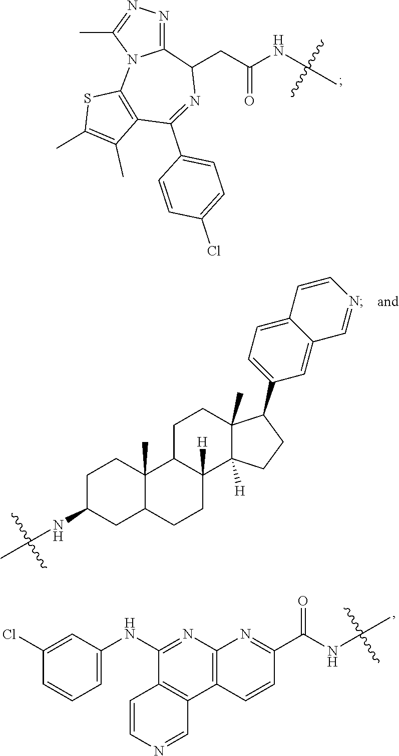
where the wiggly line represents the point of attachment to L₁.

5. (canceled)

6. The compound according to claim 1, wherein the compound of formula Ia is selected from:

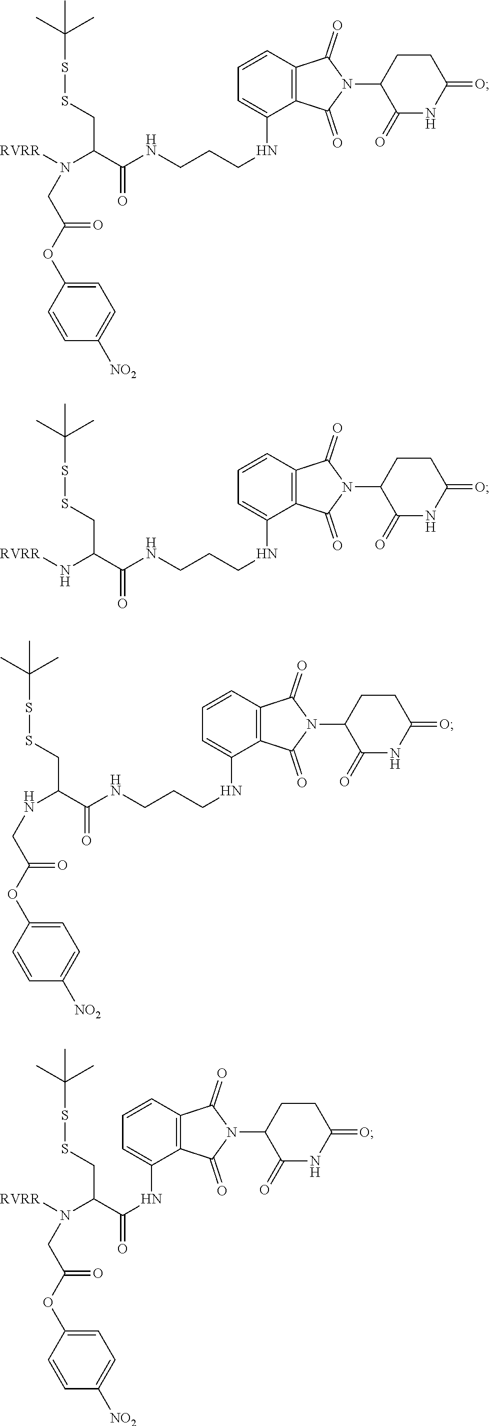

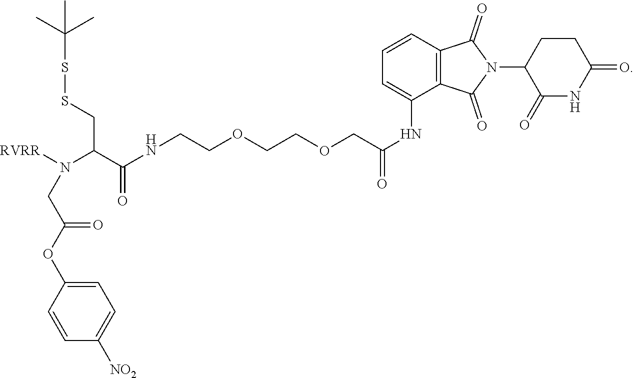

7. The compound according to claim 1, wherein when L₁ is attached to an oxygen atom on X, it is selected from: a bond; 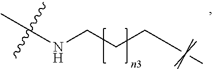
where the wiggly line represents the point of attachment to the carbonyl group next to L₁ and the crossed line represents the point of attachment to X and n3 represents from 1 to 5; 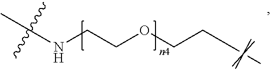
where the wiggly line represents the point of attachment to the carbonyl group next to L₁ and the crossed line represents the point of attachment to X and n4 represents from 0 to 10; or 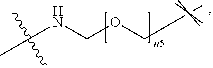
where the wiggly line represents the point of attachment to the carbonyl group next to L₁ and the crossed line represents the point of attachment to X and n5 represents from 0 to 10.

8. The compound according to claim 7, wherein X is selected from: 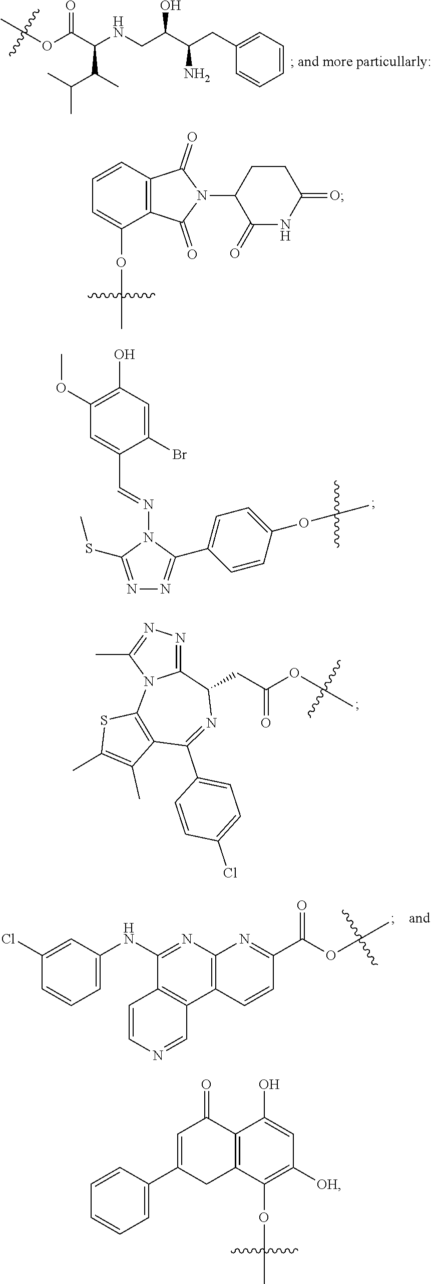
where the wiggly line represents the point of attachment to L₁.

9. The compound according to claim 1, wherein the compound of formula Ia is selected from:

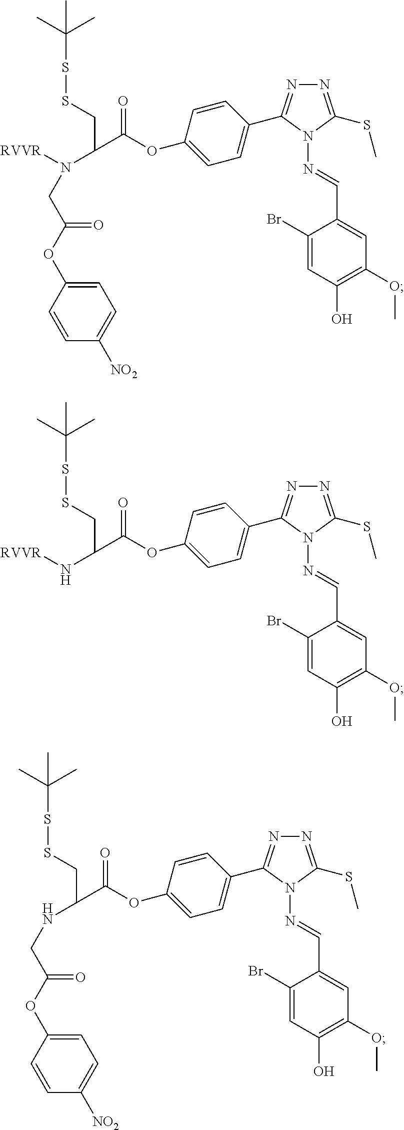

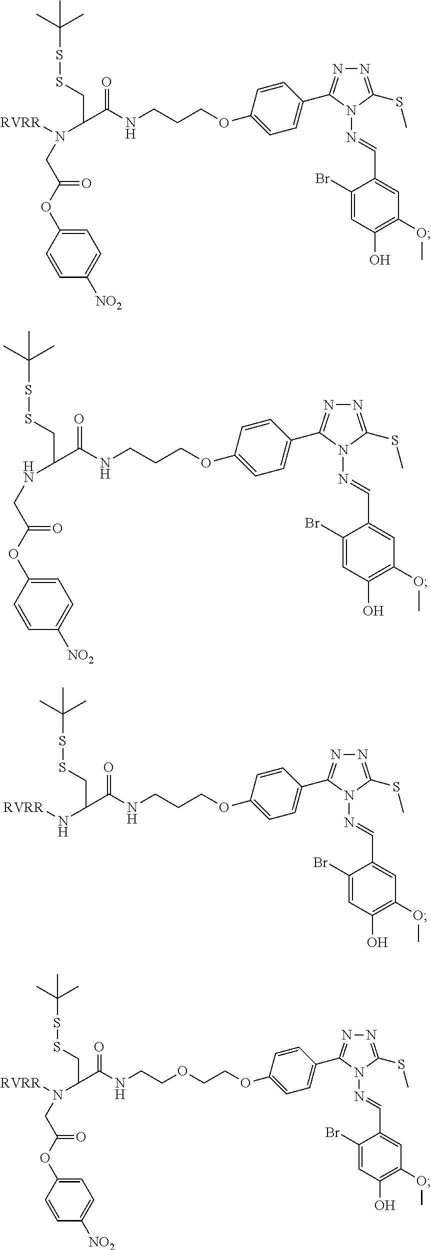

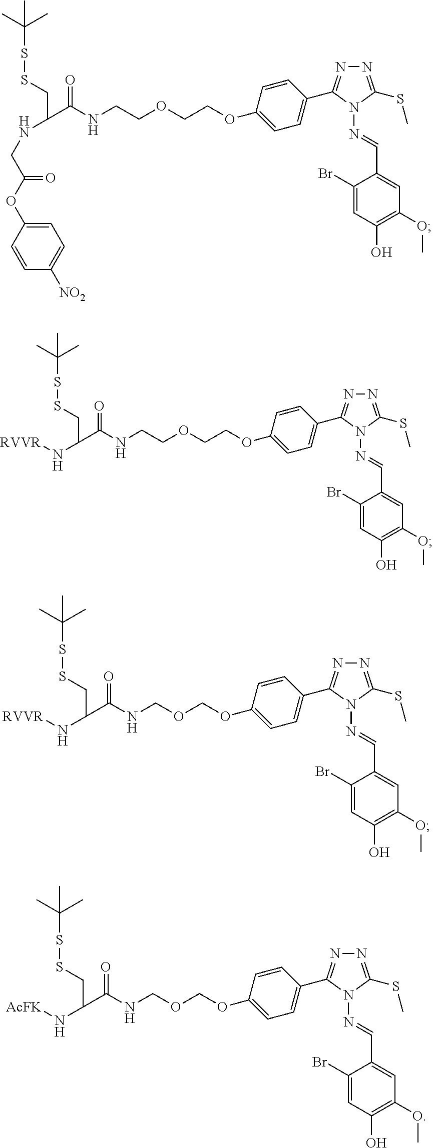

10. A compound of formula Ib: 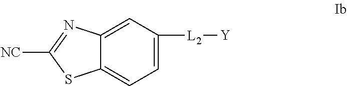
where:
L₂ is a linking group; and
Y is an E3 ligase ligand or a protein of interest ligand,
or a pharmaceutically acceptable salt or solvate thereof.

11. The compound according to claim 10, wherein when L₂ is attached to a nitrogen atom on Y, it is selected from: a bond; 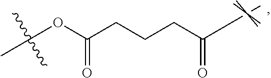
where the crossed line represents the point of attachment to Y and where the wiggly line represents the point of attachment to the rest of the molecule; 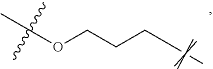
where the wiggly line represents the point of attachment to the rest of the molecule and the crossed line represents the point of attachment to Y; 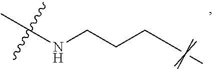
where the wiggly line represents the point of attachment to the rest of the molecule and the crossed line represents the point of attachment to Y; 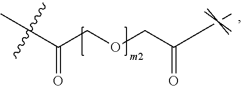
where the wiggly line represents the point of attachment to the rest of the molecule, the crossed line represents the point of attachment to Y and m₂ represents from 1 to 5.

12. The compound according to claim 10, wherein Y is selected from: 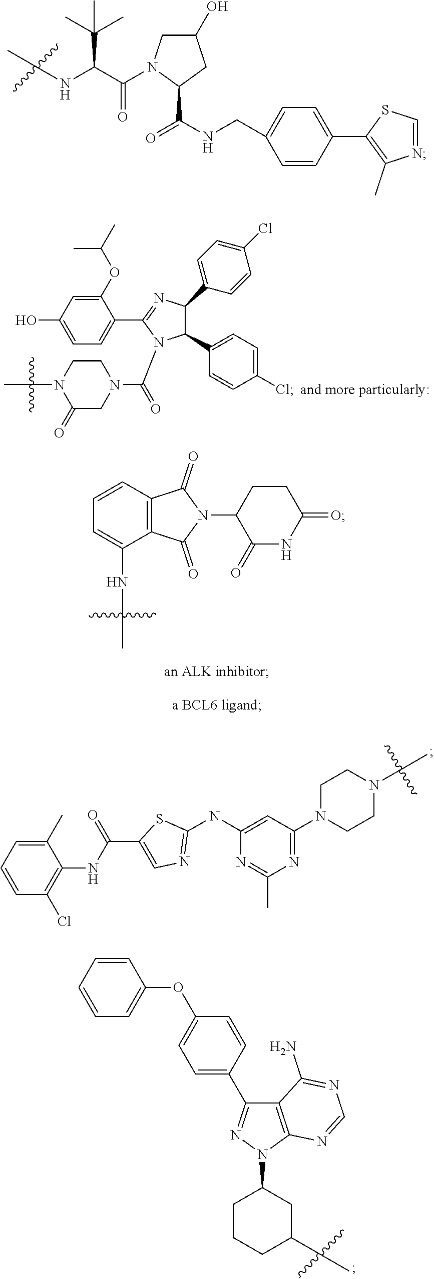 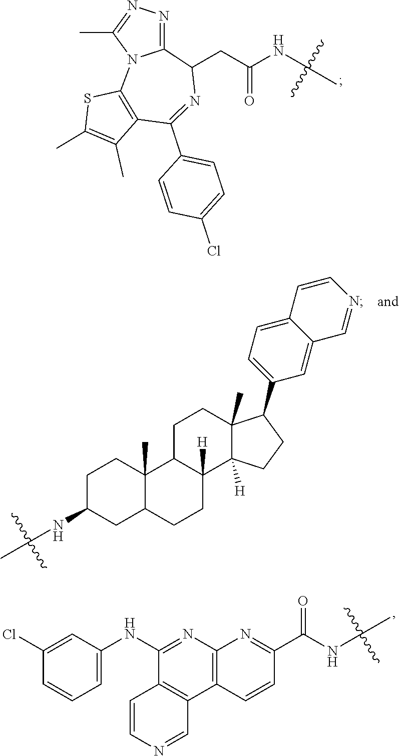
where the wiggly line represents the point of attachment to L₂.

13. (canceled)

14. The compound according to claim 10, wherein the compound of formula Ib is selected from:

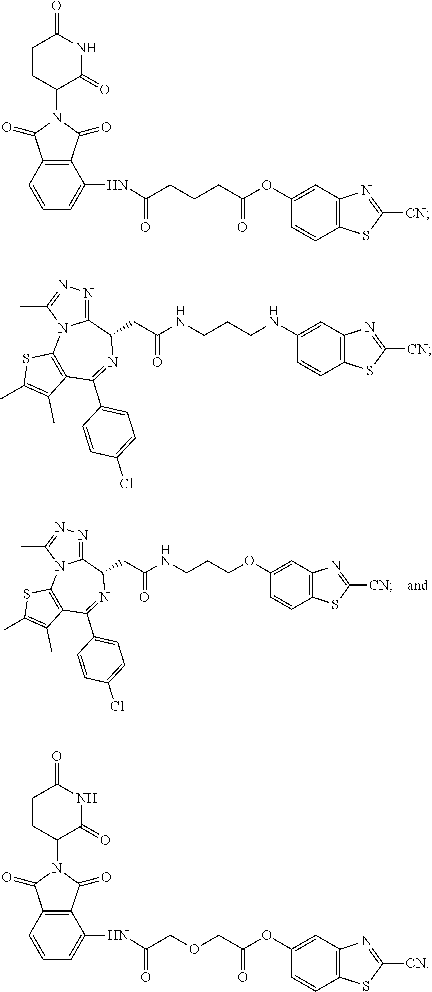

15. The compound according to claim 10, wherein when L₂ is attached to an oxygen atom on Y, it is selected from: a bond; and 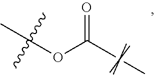
where the crossed line represents the point of attachment to Y and where the wiggly line represents the point of attachment to the rest of the molecule.

16. The compound according to claim 15, wherein Y is selected from: 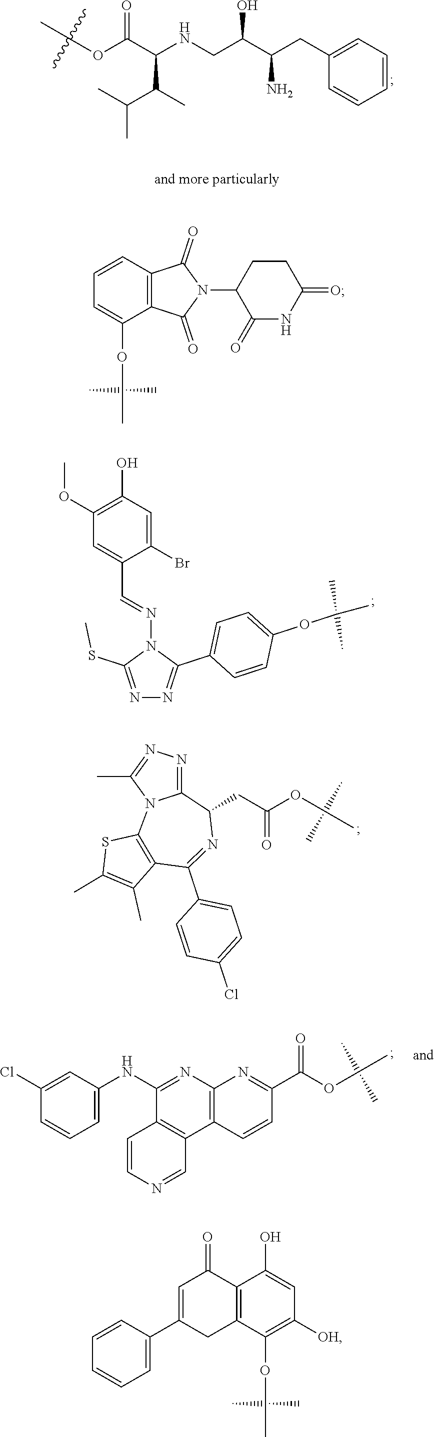
where the wiggly line represents the point of attachment to L₂.

17. The compound according to claim 10, wherein the compound of formula Ib is selected from:

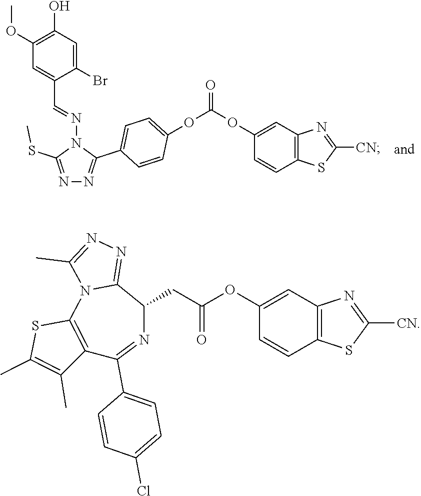 [SMILES: COc1cc(/C=N/n2c(SC)nnc2-c2ccc(OC(=O)Oc3ccc4sc(C#N)nc4c3)cc2)c(Br)cc1O ; Cc1sc2c(c1C)C(c1ccc(Cl)cc1)=N[C@@H](CC(=O)Oc1ccc3sc(C#N)nc3c1)c1nnc(C)n1-2]

18. A pharmaceutical formulation comprising one or both of:
a compound of formula Ia, or a pharmaceutically acceptable salt or solvate thereof, as described in claim 1;
a compound of formula Ib, or a pharmaceutically acceptable salt or solvate thereof, as described in claim 10;
and a pharmaceutically acceptable carrier, provided that when both of the compounds of formula Ia and formula Ib are present, one of the compounds of formula Ia and formula Ib includes an E3 ligase ligand and the other compound of formula Ia and formula Ib includes a protein of interest ligand.

19. A kit of parts comprising:
(a) a compound of formula Ia, or a pharmaceutically acceptable salt or solvate thereof, as described in claim 1; and
(b) a compound of formula Ib, or a pharmaceutically acceptable salt or solvate thereof, as described in claim 10.

20.-34. (canceled)

35. A method of treating cancer, said method comprising the step of providing a subject in need thereof a pharmaceutically effective amount of a compound of formula Ia, or a pharmaceutically acceptable salt or solvate thereof, as described in claim 1, wherein the compound of formula Ia is administered sequentially, simultaneously or concomitantly with a pharmaceutically effective amount of a compound of formula Ib, or a pharmaceutically acceptable salt or solvate thereof, as described in claim 10, or
a method of treating cancer, said method comprising the step of providing a subject in need thereof a pharmaceutically effective amount of a compound of formula Ib, or a pharmaceutically acceptable salt or solvate thereof, as described in claim 10, wherein the compound of formula Ib is administered sequentially, simultaneously or concomitantly with a pharmaceutically effective amount of a compound of formula Ia, or a pharmaceutically acceptable salt or solvate thereof, as described in claim 1.

36. (canceled)
